(19) United States
(12) Patent Application Publication
(10) Pub. No.: US 2008/0070866 A1
(43) Pub. Date: Mar. 20, 2008

(54) Chemical compounds

(75) Inventors: Hongfeng Deng, Acton, MA (US); Ghotas Evindar, Waltham, MA (US); Sylvie Bernier, Woburn, MA (US); Gang Yao, Sudbury, MA (US); Aaron Coffin, Brighton, MA (US); Hongfang Yang, Burlington, MA (US); Raksha A. Acharya, Bedford, MA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Praecis Pharmaceuticals Incorporated, Waltham, MA (US)

(21) Appl. No.: 11/888,893

(22) Filed: Aug. 1, 2007

Related U.S. Application Data
(60) Provisional application No. 60/821,112, filed on Aug. 1, 2006.
(60) Provisional application No. 60/959,216, filed on Jul. 12, 2007.
(60) Provisional application No. 60/896,442, filed on Mar. 22, 2007.
(60) Provisional application No. 60/827,923, filed on Oct. 3, 2006.

(51) Int. Cl. A61K 31/433; A61K 31/421; A61K 31/67; A61P 21/00; A61P 43/00; C07D 263/06; C07D 277/24; C07D 285/08; C07F 9/06

(52) U.S. Cl. 514/91; 514/361; 514/365; 514/374; 548/119; 548/128; 548/205; 548/215

(57) ABSTRACT

The invention provides compounds of formula I and formula II, their preparation, and their use as pharmaceutically active immunosuppressive agents for the treatment of autoimmune disorders, organ transplant rejection, disorders associated with an activated immune system, as well as other disorders modulated by lymphopenia or S1P receptors.

### RELATED APPLICATIONS

This application is related and claims priority to U.S. provisional application Ser. No. 60/821,112, filed Aug. 1, 2006, U.S. provisional application Ser. No. 60/827,923, filed Oct. 3, 2006, U.S. provisional application Ser. No. 60/896,442, filed Mar. 22, 2007 and U.S. provisional application Ser. No. 60/959,216, filed Jul. 12, 2007, the entire contents of each of which are incorporated herein by this reference.

### BACKGROUND OF THE INVENTION

The sphingosine-1-phosphate (S1P) receptors 1-5 constitute a family of seven transmembrane G-protein coupled receptors. These receptors, referred to as S1P-1 to S1P-5, are activated via binding by sphingosine-1-phosphate, which is produced by the sphingosine kinase-catalyzed phosphorylation of sphingosine. S1P receptors are cell surface receptors involved in a variety of cellular processes, including cell proliferation and differentiation, cell survival, cell invasion, lymphocyte trafficking, and cell migration. Sphingosine-1-phosphate is found in plasma and a variety of other tissues, and exerts autocrine and paracrine effects, including regulating the secretion of growth factors.

Administration of S1P to an animal results in sequestration of lymphocytes into the lymph nodes and Peyers patches without causing lymphocyte depletion. This activity, which is of potential utility in treating diseases or conditions associated with inappropriate immune response, including transplant rejection, autoimmune diseases, as well as other disorders modulated by lymphocyte trafficking, is believed to proceed via activation of the S1P-1 receptor. Administration of S1P in vivo has been shown to cause hypotension and bradycardia, which are believed to be due to signaling through one or more of the other S1P receptors, i.e. S1P-2 to S1P-5. Accordingly, there is a need for compounds which are potent and selective agonists of the S1P-1 receptor.

### SUMMARY OF THE INVENTION

These and other needs are met by the present invention. In some aspects, the present invention is directed to a compound of formula I

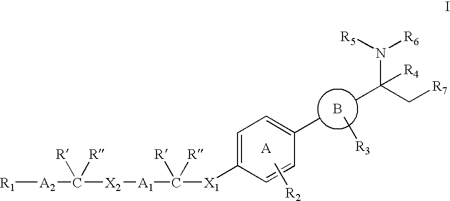

or a pharmaceutically acceptable salt thereof, wherein:

R₁ is hydrogen, halogen, cyano, alkyl, aryl, heteroaryl, cycloalkyl, heterocycloalkyl, aralkyl, heteroalykl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkyl-SO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 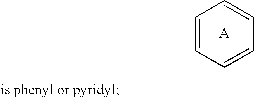 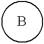
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;
R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein

Z′ is hydroxyl or halogen;

Z″ is H or halogen;

Rₚ₁ and Rₚ₂ are each independently hydrogen, C₁-C₆-alkyl, aryl, or one of the following groups:

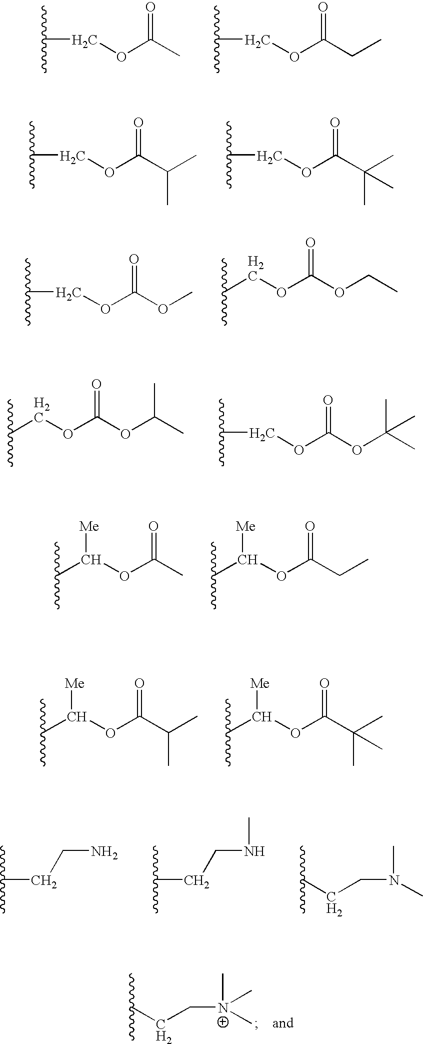

Y is heterocyclo or heteroaryl.

In some aspects, the present invention is directed to a compound of formula II

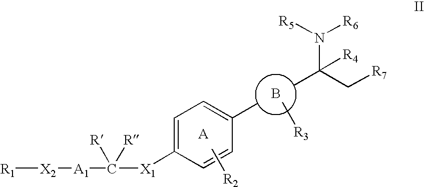

or a pharmaceutically acceptable salt thereof, wherein:

R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 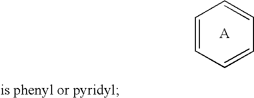 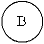
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;

R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein

Z′ is hydroxyl or halogen;

Z″ is H or halogen;

Rₚ₁ and Rₚ₂ are each independently hydrogen, C₁-C₆-alkyl, aryl, or one of the following groups:

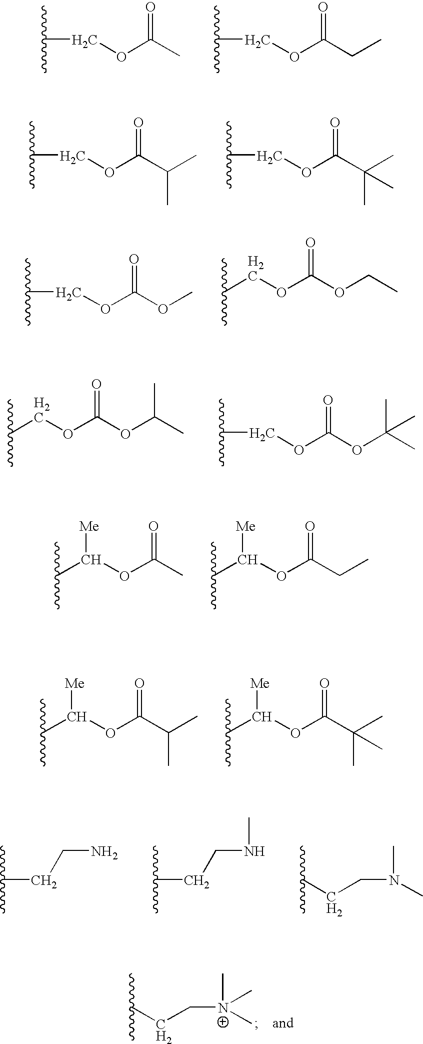

Y is heterocyclo or heteroaryl.

In some aspects, the present invention is directed to the compounds of the following table:

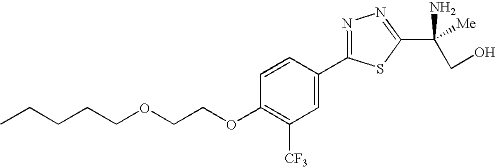

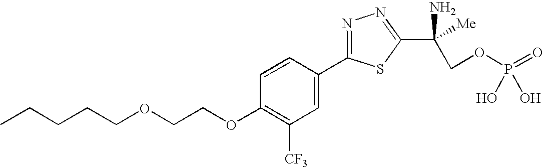

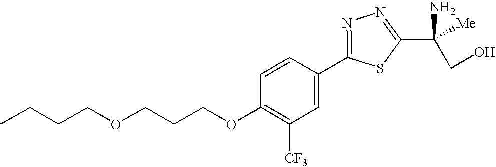

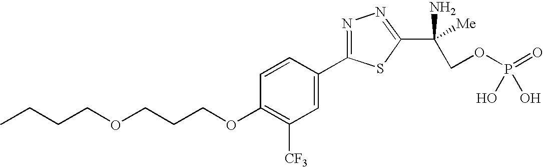

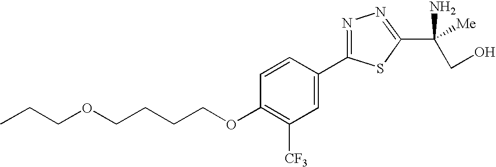

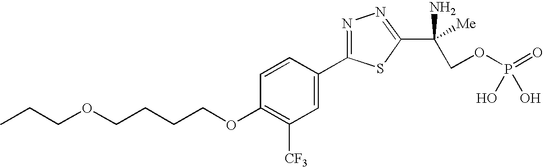

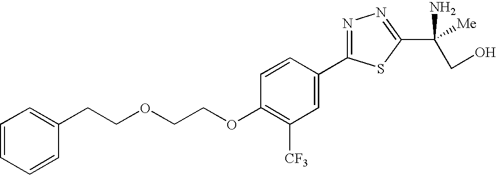

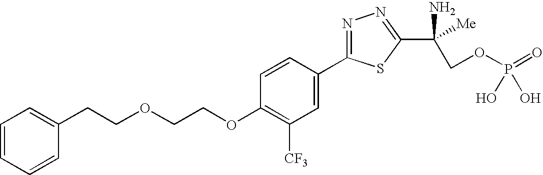

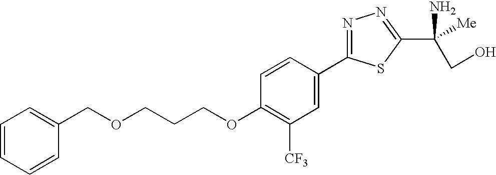

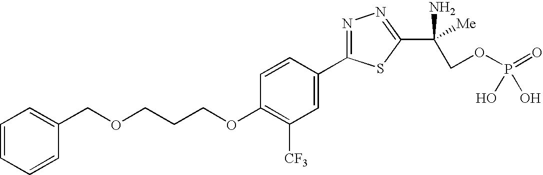

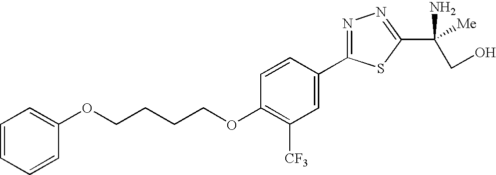

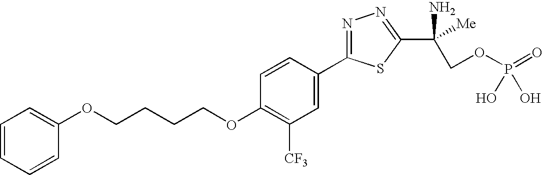

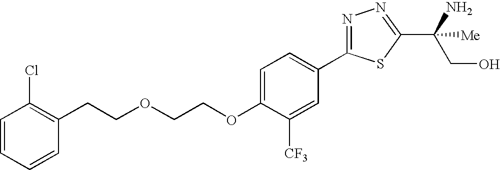

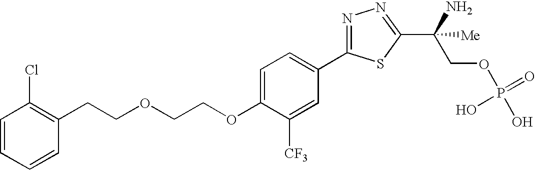

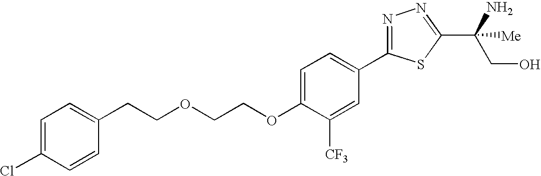

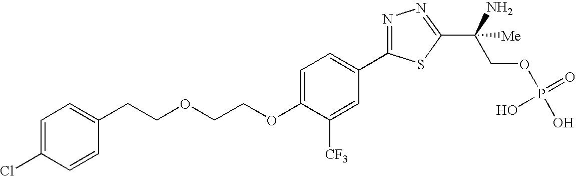

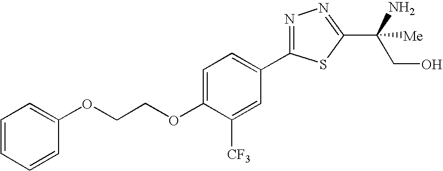

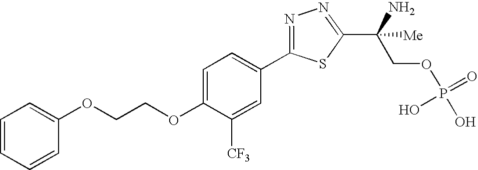

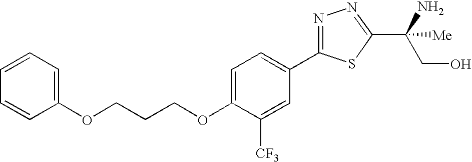

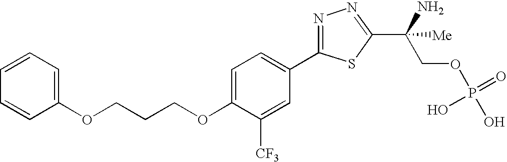

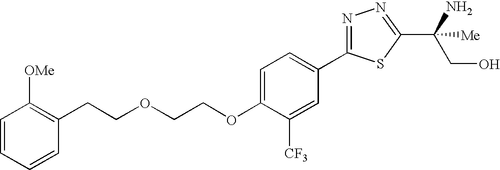

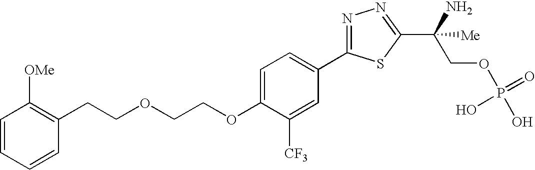

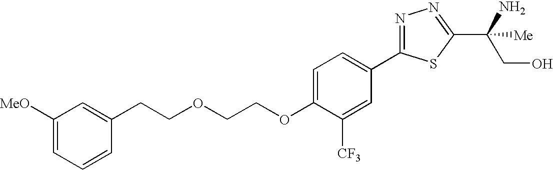

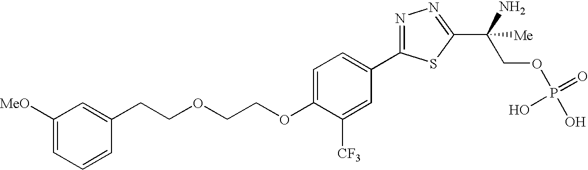

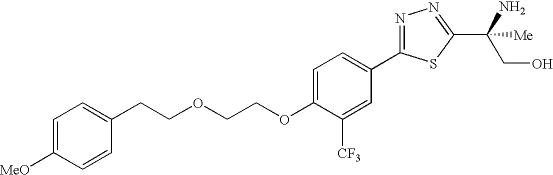

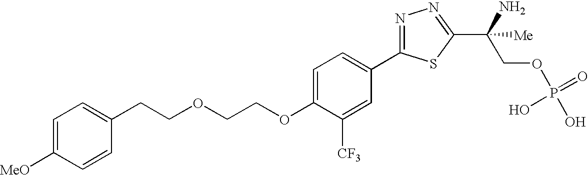

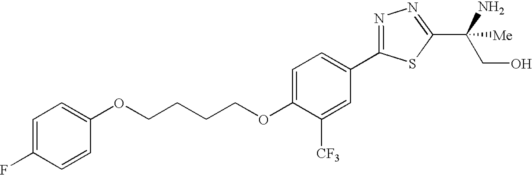

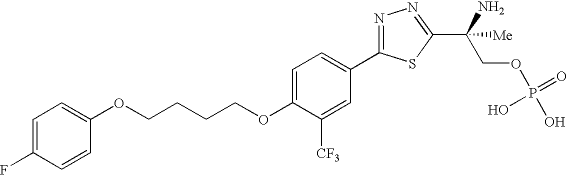

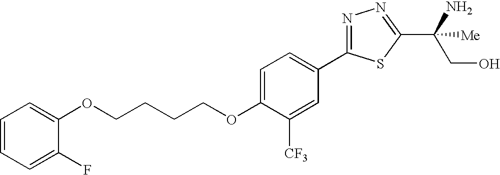

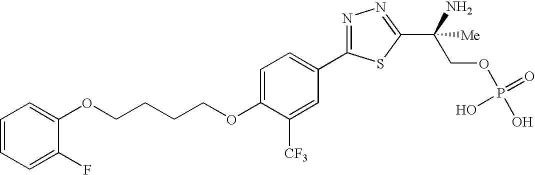

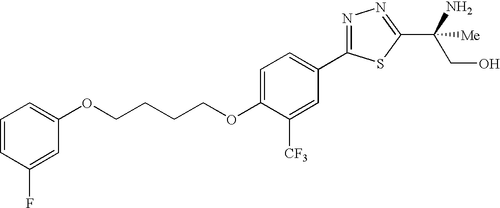

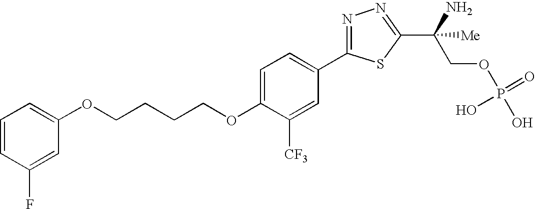

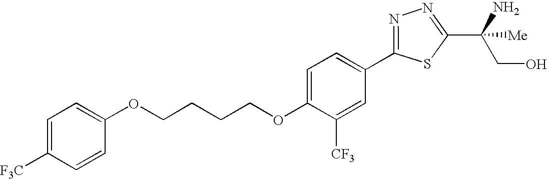

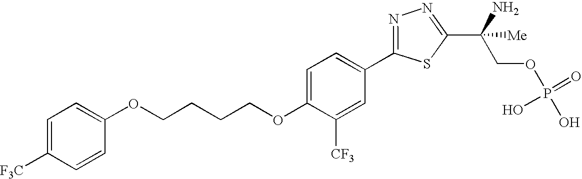

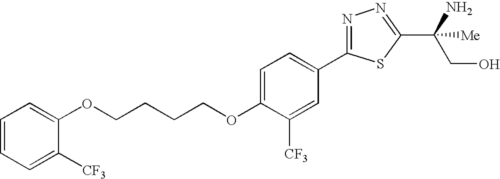

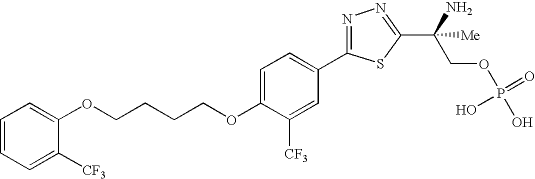

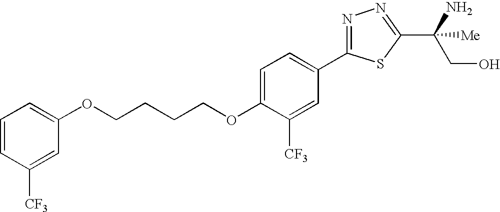

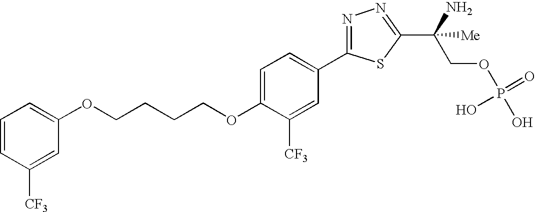

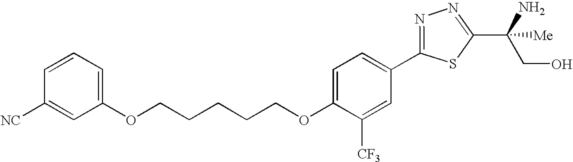

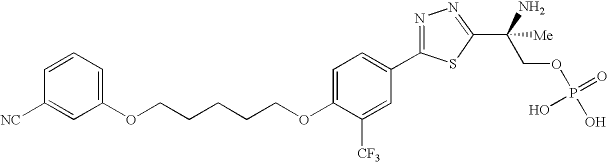

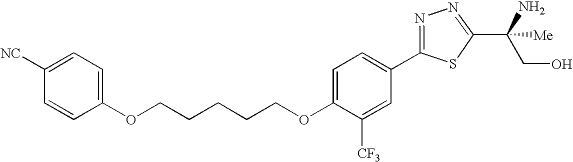

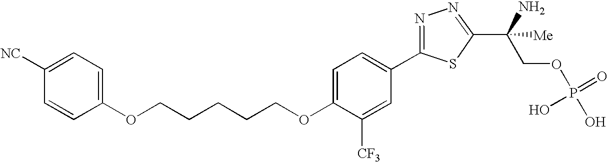

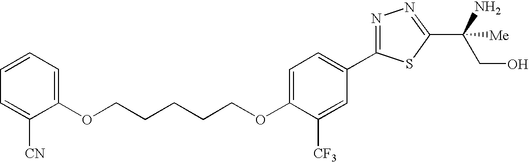

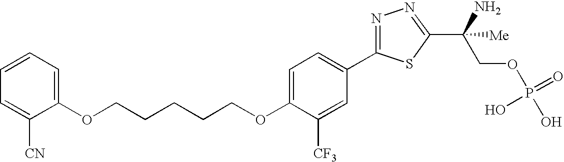

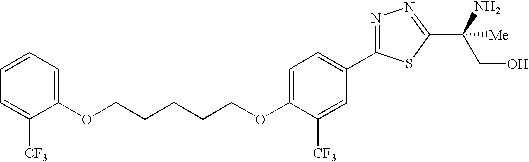

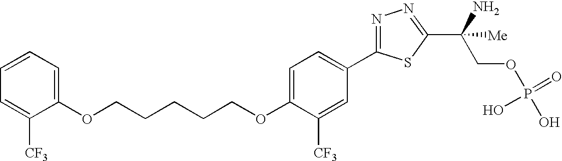

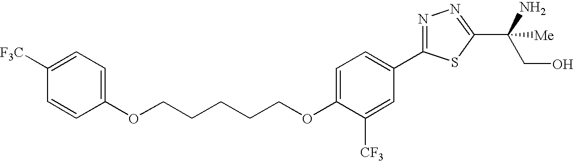

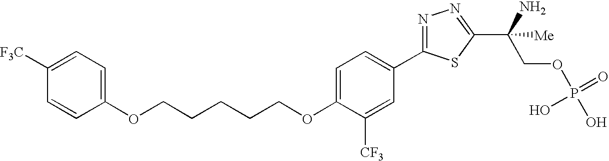

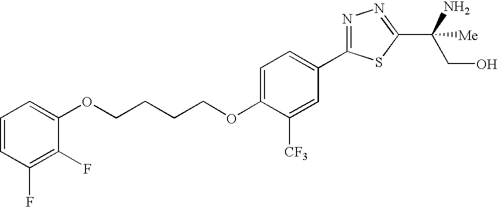

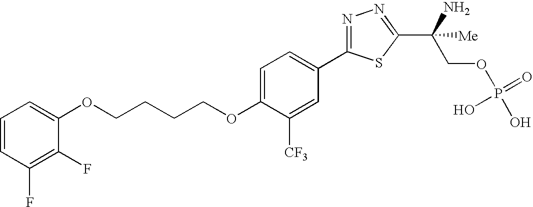

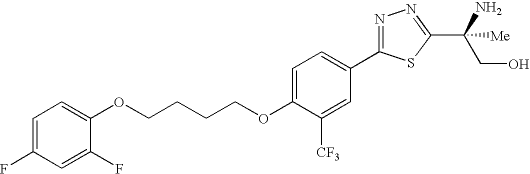

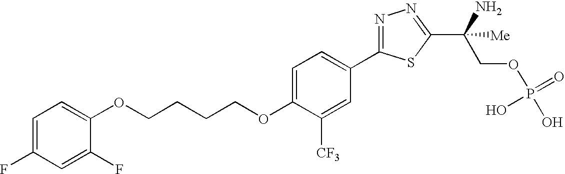

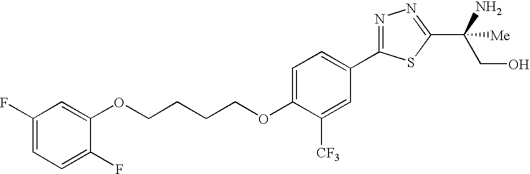

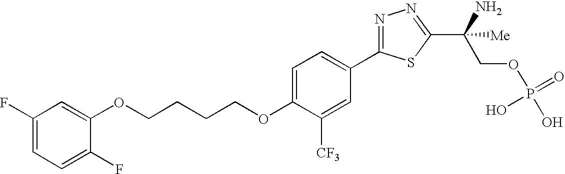

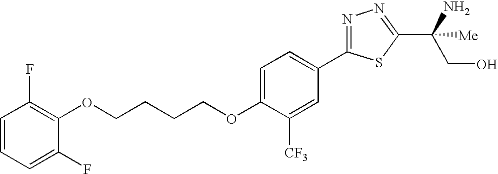

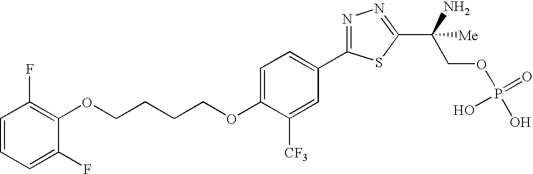

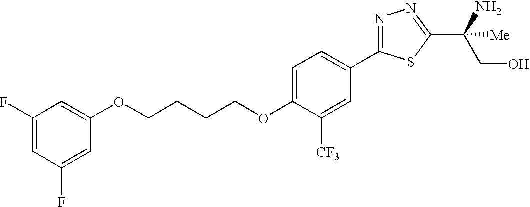

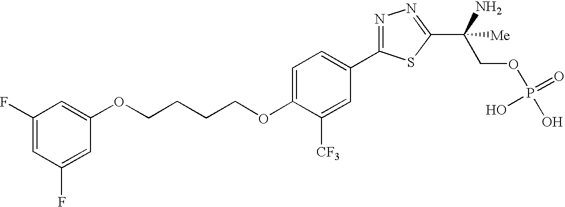

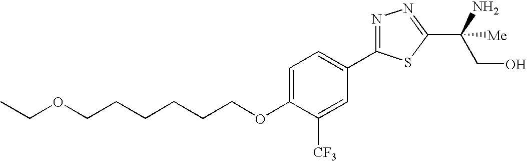

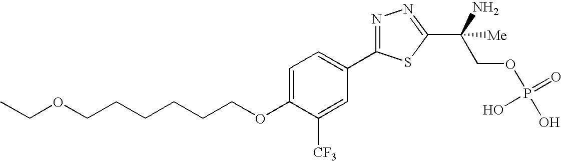

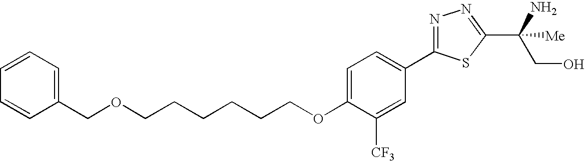

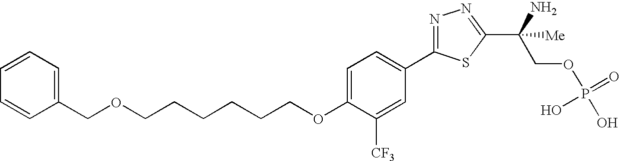

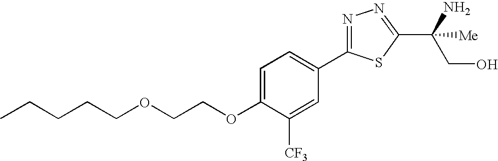

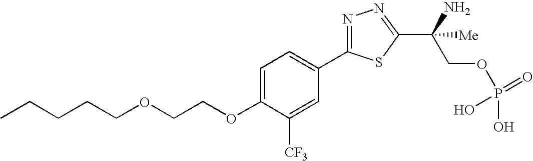

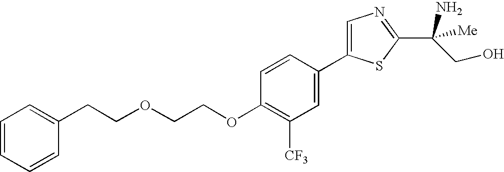

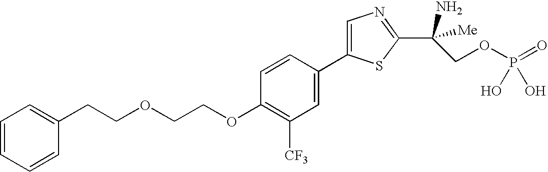

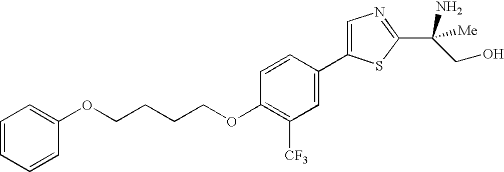

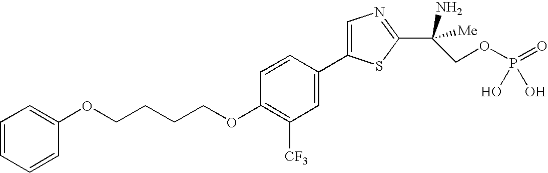

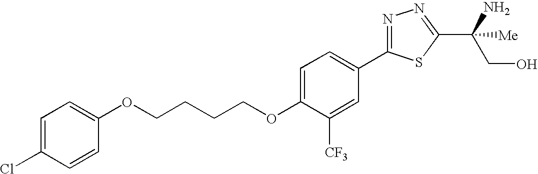

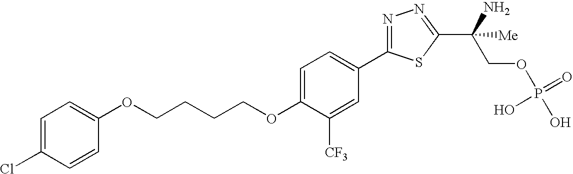

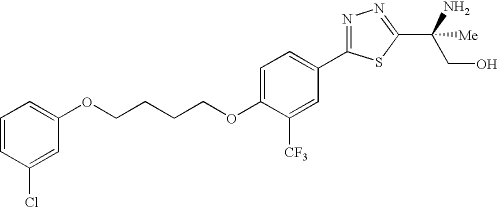

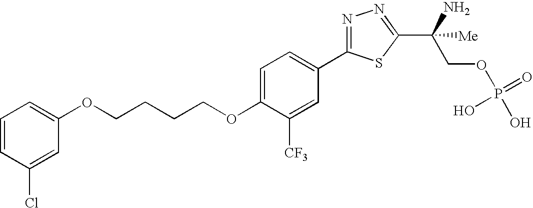

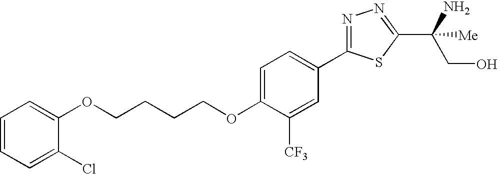

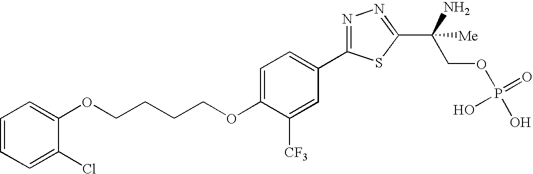

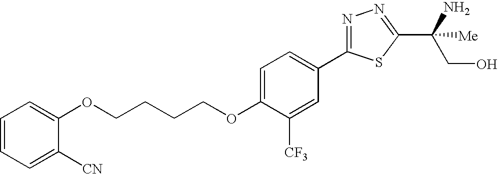

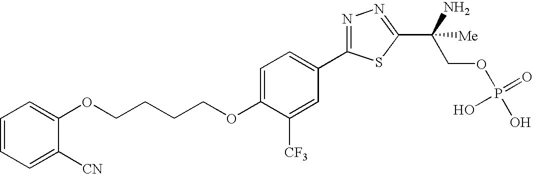

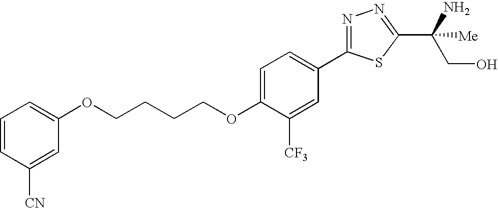

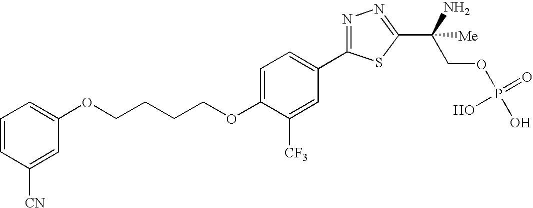

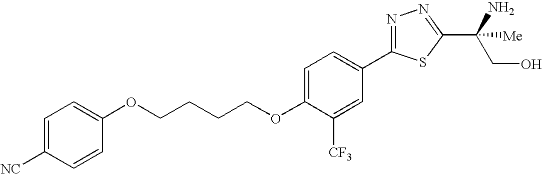

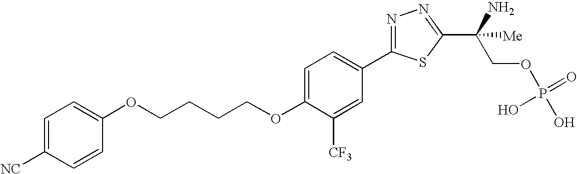

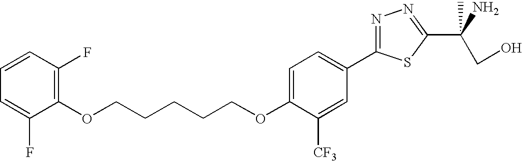

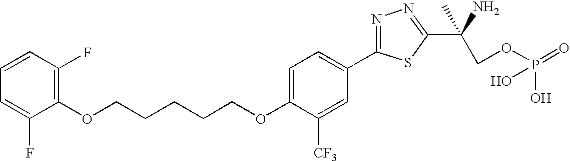

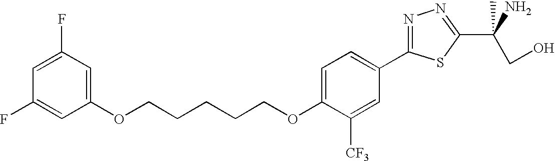

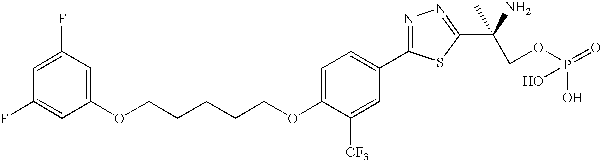

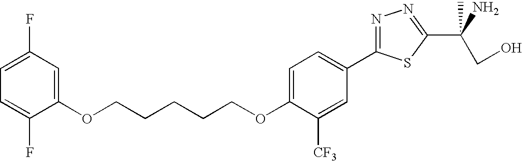

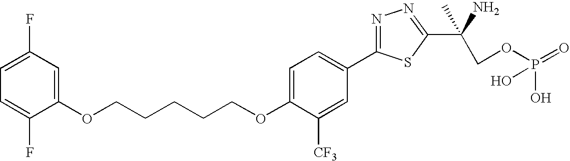

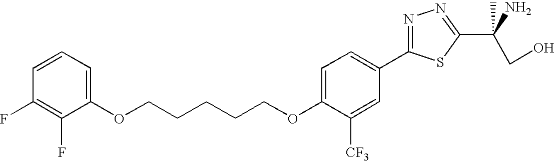

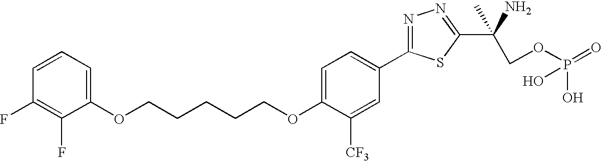

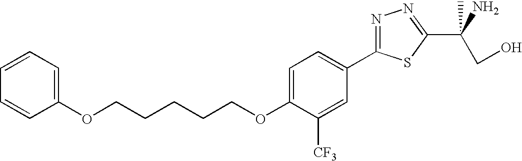

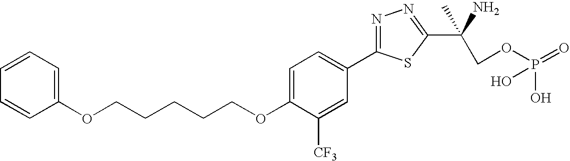

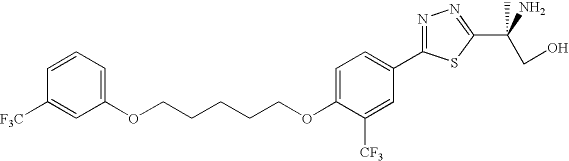

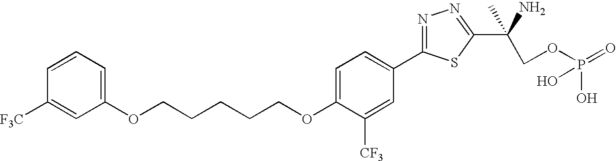

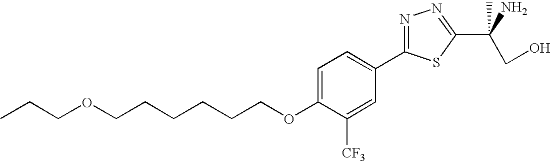

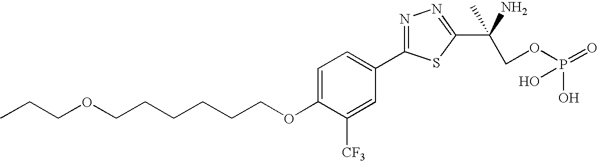

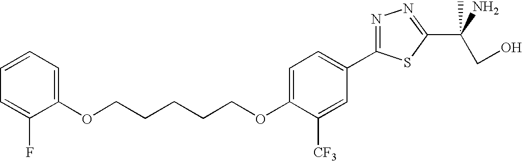

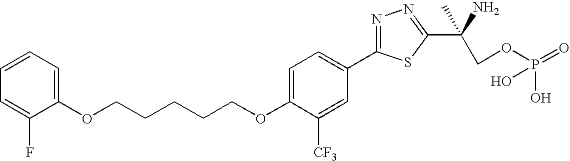

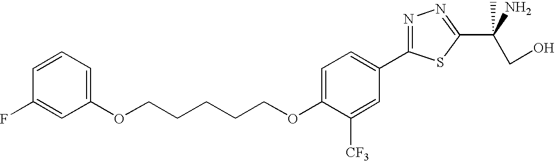

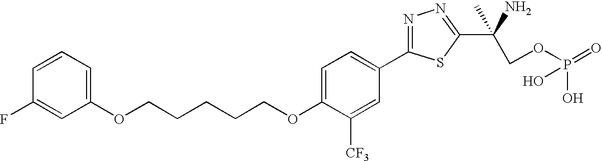

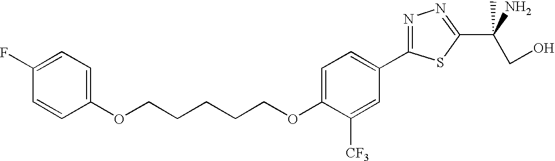

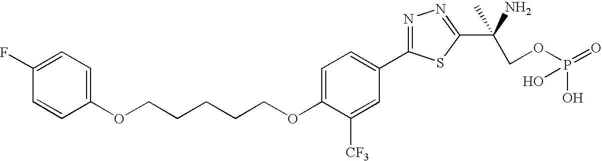

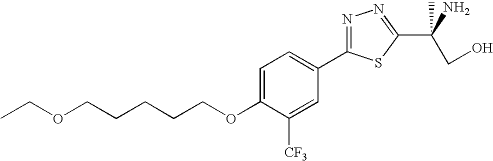

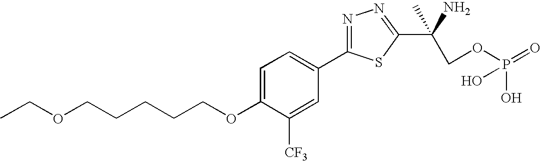

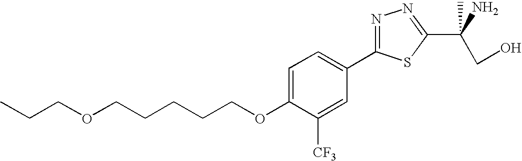

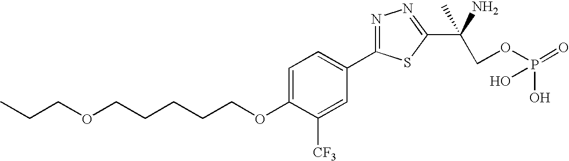

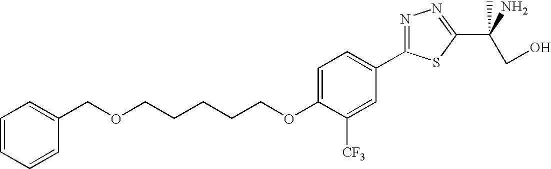

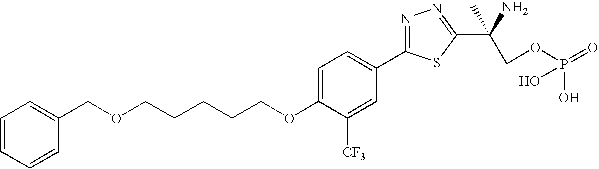

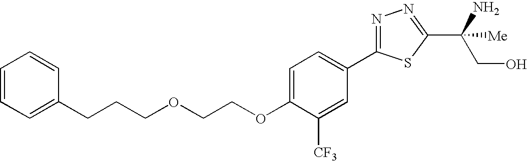

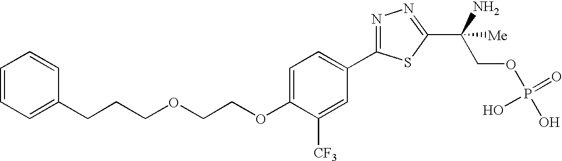

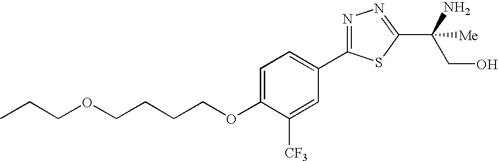

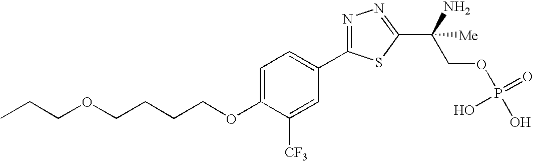

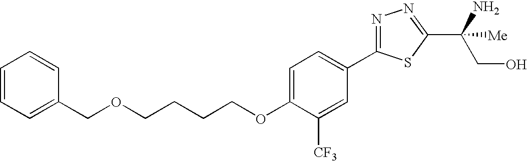

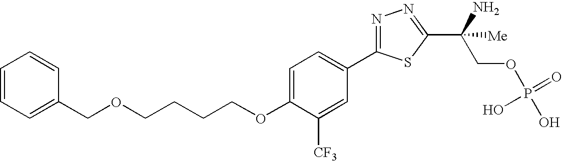

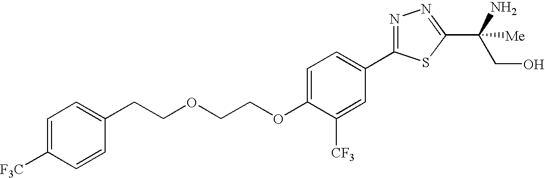

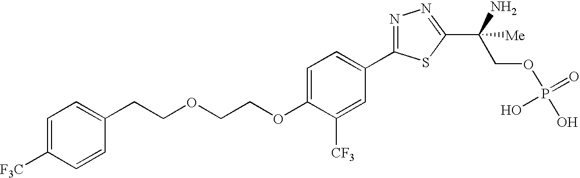

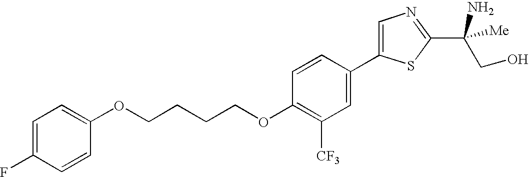

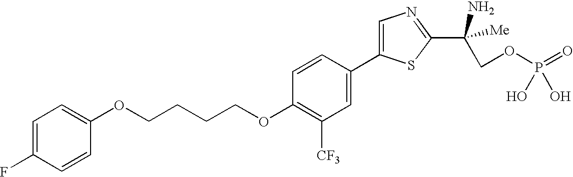

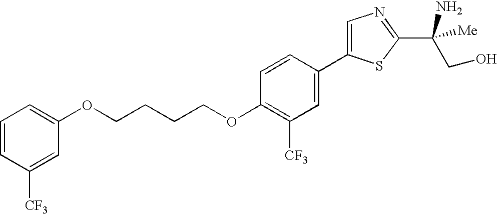

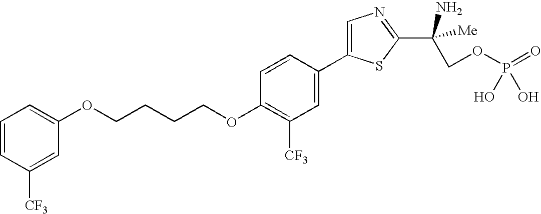

as well as pharmaceutically acceptable salts, phosphate derivatives, phosphate mimics, or phosphate precursor analogs thereof.

In some aspects, the present invention is directed to a compound of formula III:

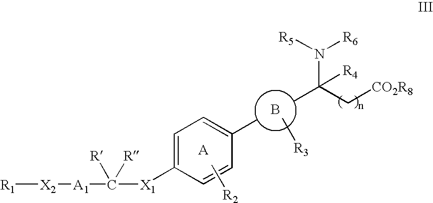

or a pharmaceutically acceptable salt thereof, wherein:

R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 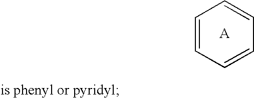 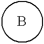
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;

R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

n is 0, 1, or 2;

R₈ is hydrogen, alkyl, or aryl.

In some aspects, the present invention is directed to the compounds of the following table:

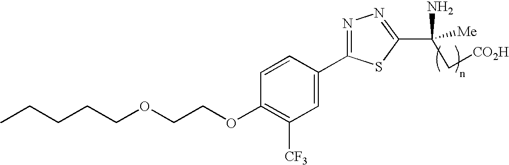

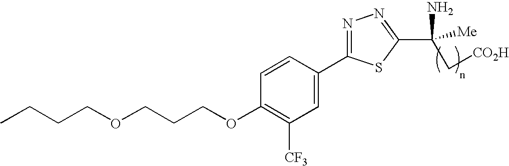

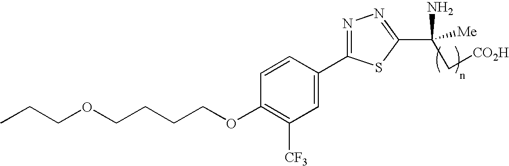

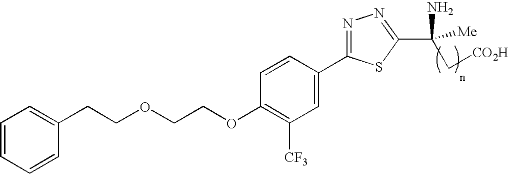

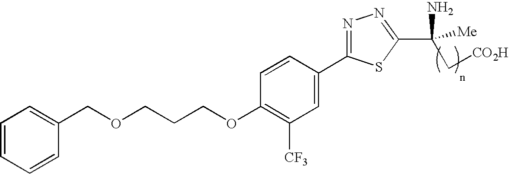

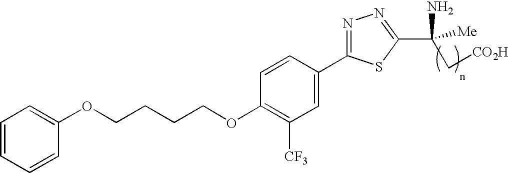

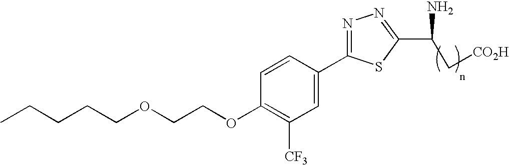

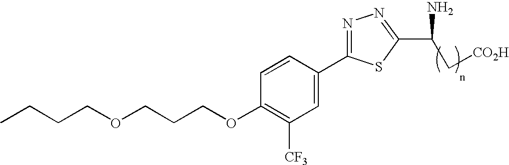

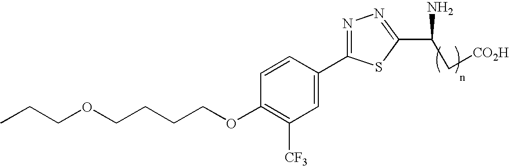

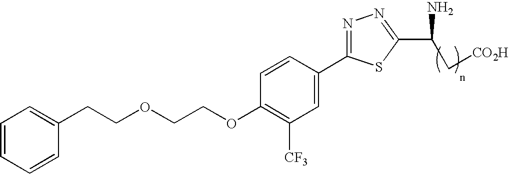

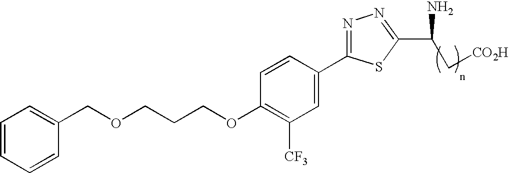

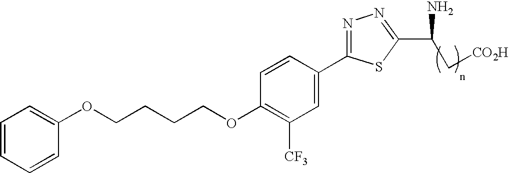

wherein n for each compound is 0, 1, or 2, as well as pharmaceutically acceptable salts, phosphate derivatives, phosphate mimics, or phosphate precursor analogs thereof.

In some aspects, the present invention is directed to a method of treating a sphingosine 1-phosphate associated disorder in a subject in need thereof comprising administering to the subject a therapeutically safe and effective amount of a compound of any of formulas I, II or III, or a pharmaceutically acceptable salt, phosphate derivative, phosphate mimic, or phosphate precursor analog thereof, such that the sphingosine 1-phosphate associated disorder is treated.

In some aspects, the present invention is directed to a method of treating an autoimmune disorder comprising administering to a subject in need thereof a pharmaceutically acceptable amount of a compound of any of formulas I, II or III, such that the autoimmune disorder is treated.

In some aspects, the present invention is directed to a method treating transplant rejection comprising administering to a subject in need thereof a pharmaceutically acceptable amount of a compound of any of formulas I, II or III, such that the transplant rejection is treated.

In some aspects, the present invention is directed to a compound of any of formulas I, II or III for use as a therapeutic substance.

In some aspects, the present invention is directed to a compound of any of formulas I, II or III for use in the treatment of sphingosine associated disorders. In some aspects, the present invention is directed to a compound of any of formulas I, II or III for use in the treatment of multiple sclerosis.

In some aspects, the present invention is directed to a compound of any of formulas I, II or III for use in the manufacture of a medicament for use in the treatment of sphingosine associated disorders. In some aspects, the present invention is directed to a compound of any of formulas I, II or III for use in the manufacture of a medicament for the treatment of multiple sclerosis.

In some aspects, the present invention is directed to a pharmaceutical composition comprising a compound of any of formulas I, II or III and a pharmaceutically acceptable carrier.

In some aspects, the present invention is directed to a process for making any of the compounds described herein.

### DETAILED DESCRIPTION OF THE INVENTION

### Definitions

The following definitions are used, unless otherwise described.

“Halogen” or “halo” means fluoro (F), chloro (Cl), bromo (Br), or iodo (I).

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl’ used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “cycloalkyl” used alone or as suffix or prefix, refers to a saturated or partially unsaturated monovalent ring-containing hydrocarbon radical comprising at least 3 up to about 12 carbon atoms.

The term “aryl” used alone or as suffix or prefix, refers to a monovalent hydrocarbon radical having one or more polyunsaturated carbon rings having aromatic character, and comprising 5 up to about 14 carbon atoms.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The terms “heterocyclic group”, “heterocyclic moiety”, “heterocyclic”, or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heteroaryl” used alone or as a suffix or prefix, refers to a heterocyclyl having aromatic character.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles (heteroaryl groups), for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “six-membered” used as prefix refers to a group having a ring that contains six ring atoms.

The term “five-membered” used as prefix refers to a group having a ring that contains five ring atoms.

A five-membered heteroaryl ring is a heteroaryl with a ring having five ring atoms wherein 1, 2 or 3 ring atoms are independently selected from N, O and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S. Exemplary six-membered ring heteroaryls are pyridyl, pyrazinyl, pyrimidinyl, triazinyl and pyridazinyl.

The term “aralkyl” refers to an alkyl group substituted with an aryl group.

The term “heteroaralkyl” refers to an alkyl group substituted with an heteroaryl group.

Unless otherwise specified, the term “substituted”, when used as a prefix, refers to a structure, molecule or group, wherein one or more hydrogens are replaced with one or more alkyl groups, or one or more chemical groups containing one or more heteroatoms selected from N, O, S, F, Cl, Br, I, and P. Exemplary chemical groups containing one or more heteroatoms include heterocyclyl, —NO₂, —O-alkyl, halo, —CF₃, —CO₂H, —CO₂R, —NH₂, —SH, —NHR, —NR₂, —SR, —SO₃H, —SO₂R, —S(O)R, —CN, —OH, —C(O)NR₂, —NRC(O)R, oxo (═O), imino (═NR), thio (═S), and oximino (═N—OR), wherein each “R” is alkyl as defined above. For example, substituted phenyl may refer to nitrophenyl, pyridylphenyl, methoxyphenyl, chlorophenyl, aminophenyl, an so on, wherein the nitro, pyridyl, methoxy, chloro, and amino groups may replace any suitable hydrogen on the phenyl ring. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc.

The term “alkoxy” used alone or as a suffix or prefix, refers to radicals of the general —O-alkyl, Exemplary alkoxy groups includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers —NH₂.

The term “alkylamino” used alone or as a suffix or prefix, refers —NH(alkyl). The term “dialkylamino” used alone or as a suffix or prefix, refers —NH(alkyl)₂.

“Acyl” used alone, as a prefix or suffix, means —C(O)—R, wherein R hydrogen, hydroxyl, amino, alkylamino, dialkylamino, or alkoxy, any of which may be substituted as provided by the definition of “substituted” given above. Acyl groups include, for example, acetyl, propionyl, benzoyl, phenyl acetyl, carboethoxy, and dimethylcarbamoyl.

Some of the compounds in the present invention may exist as stereoisomers, including enantiomers, diastereomers, and geometric isomers. All of these forms, including (R), (S), epimers, diastereomers, cis, trans, syn, anti, solvates (including hydrates), tautomers, and mixtures thereof, are contemplated in the compounds of the present invention.

The invention also relates to salts of the compounds of the invention and, in particular, to pharmaceutically acceptable salts. A “pharmaceutically acceptable salt” is a salt that retains the desired biological activity of the parent compound and does not impart any undesired toxicological effects. The salts can be, for example, salts with a suitable acid, such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as ammonium, sodium, potassium, lithium, zinc, copper, barium, bismuth, calcium, and the like; or organic cations such as tetralkylammonium and trialkylammonium cations. Combinations of the above salts are also useful. Salts of other acids and/or cations are also included, such as salts with trifluoroacetic acid, chloroacetic acid, and trichloroacetic acid. The invention also includes different crystal forms, hydrates, and solvates of the compounds of the invention.

The terms “phosphate precursor” and “phosphate precursor analog,” as used herein, refer to substituent moieties in the compounds of the invention that may be directly phosphorylated in vivo, or which may be cleaved in vivo to reveal a moiety that may then be phosphorylated in vivo. In certain embodiments, the phosphate precursor may be L₁-O—H or L₁-O-L₂, wherein L₁ is a linking moiety and L₂ is a labile moiety. Exemplary embodiments of the phosphate precursor, include but are not limited to -alkyl-OH, -halo-alkyl-OH, alkoxy-OH, -alkyl-OCOR^{a}, -halo-alkyl-OCOR^{a}, -alkoxy-OCOR^{a}, -alkyl-OC(O)NR^{a}R^{b}, -halo-alkyl-OC(O)NHR^{a}R^{b}, -alkoxy-OC(O)NR^{a}R^{b}, —(CH₂)_{q}CO₂R^{c}, and —(CH₂)ₙCH₂═CHC(O)OR^{c}, wherein

q is an integer between 0 and 4;

R^{a} and R^{b} are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R^{c} is selected from the group consisting of hydrogen, straight chain or branched C₁-C₆-alkyl, straight chain or branched halo-C₁-C₆-alkyl, substituted or unsubstituted aryl group, or one of the following groups.

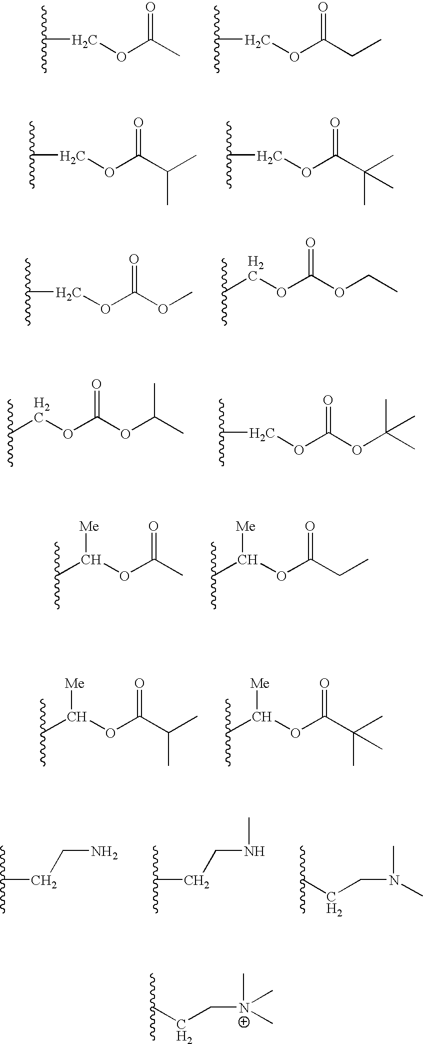

The “linking moiety,” may contain 1-8 atoms or may be a bond, and serves as the connection point through which the phosphate mimic, phosphate derivative, or phosphate precursor substituent moieties are linked to the remaining structure of the compounds of the invention. In certain embodiments, the linking moiety may include, but is not limited to, substituted or unsubstituted alkyl (e.g., methylene chains), substituted or unsubstituted alkenyl (e.g., n-alkenes), substituted or unsubstituted alkynyl, substituted or unsubstituted halo-alkyl, substituted or unsubstituted alkoxy, and substituted or unsubstituted halo-alkoxy. In specific embodiments, the linking moiety may be carbonyl derivatized.

The language “labile moiety” refers to a moiety that is subject to cleavage, for instance, by hydrolysis or enzymatic degradation. In certain embodiments, the labile moiety is an ester moiety, which may result in a carboxylate or hydroxyl derivative, depending on the orientation of the ester functionality in the molecule prior to cleavage.

The term “phosphate derivative” refers to substituent moieties in the compounds of the invention that contain a phosphate or phosphate ester group. When a compound of the invention containing a phosphate derivative is administered to a subject, the compound may act as is in vivo or the phosphate derivative (within the compound) may be cleaved and then re-phosphorylated in vivo leading to an active compound. In certain embodiments, the phosphate derivative may be selected from the group consisting of —(CH₂)_{q} OPO₂R^{d}R^{e}, —(CH₂)_{q}OPO₃R^{d}R^{e}, and —(CH₂)_{q}OPO₂(S)R^{d}R^{e}, wherein

q is an integer between 0 and 4; and

R^{d} and R^{e} are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The term “phosphate mimic” refers to substituent moieties in the compounds of the invention in which a phosphate substrate has been replaced with a non-hydrolyzable functional group, resulting in a moiety that mimics the biological function of a phosphate or phosphate ester moiety. In certain embodiments, the phosphate mimic is -L₁-Z₂, wherein L₁ is a linking moiety and Z₂ is a non-hydrolyzable moiety covalently bonded, to L₁. In certain embodiments, the phosphate mimic is selected from the group consisting of —(CH₂)_{q}CH₂PO₃R^{d}R^{e}, and —(CH₂)_{q}C(Y₁)(Y₂)PO₃R^{d}R^{e}, wherein

q is an integer between 0 and 4;

Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R^{d} and R^{e} are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The language “non-hydrolyzable moiety” is art-recognized, and refers to moieties containing bonds, such as carbon-phosphorous bonds, that are not hydrolyzable in vivo.

### COMPOUNDS OF THE INVENTION

In some aspects, the present invention is directed to compounds of formula I.

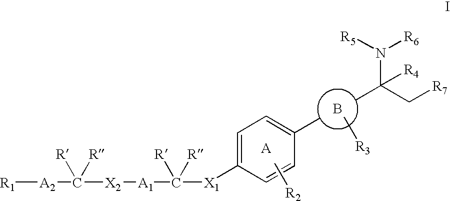

wherein:

R₁ is hydrogen, halogen, cyano, alkyl, aryl, heteroaryl, cycloalkyl, heterocycloalkyl, aralkyl, heteroalykl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkyl-SO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 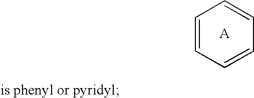 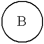
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;

R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein

Z′ is hydroxyl or halogen;

Z″ is H or halogen;

Rₚ₁ and Rₚ₂ are each independently hydrogen, C₁-C₆-alkyl, aryl, or one of the following groups:

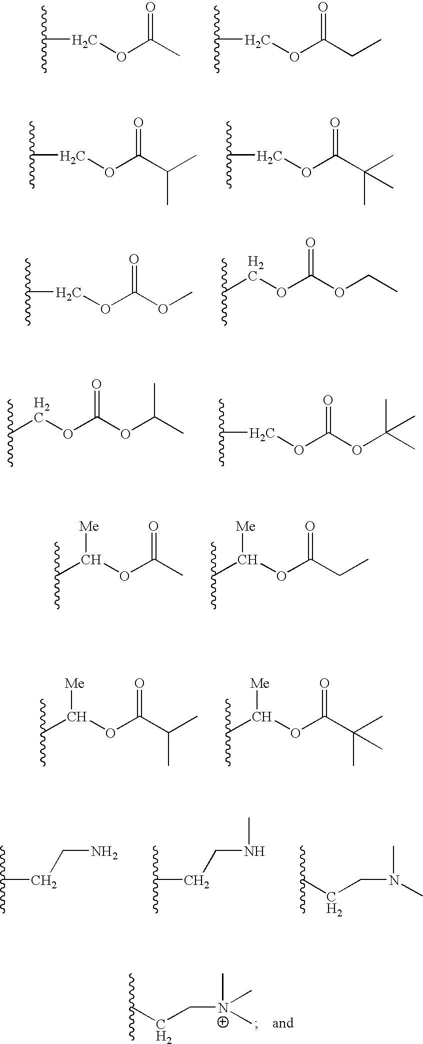

Y is heterocyclo or heteroaryl.

In some embodiments, R₁ is aryl, optionally substituted with 1, 2, or 3 groups selected from halo, alkyl, haloalkyl, —CF₃, —CN, —OH, or —O-alkyl. In some embodiments, R₁ is aryl, e.g., phenyl, optionally substituted with 1 or 2 groups selected from —CF₃, —CN, —OMe, —Cl or —F. In some embodiments, R₁ is hydrogen. In other embodiments, R₁ is phenyl. In other embodiments, R₁ is pyridyl. In still other embodiments, R₁ is thiophenyl. In other embodiments, R₁ is cyclohexyl. In yet other embodiments, R₁ is cyclopentyl.

In some embodiments, A₁ is n-octyl. In other embodiments, A₁ is n-heptyl. In some embodiments, A₁ is a C₁₋₅ alkylene. In still other embodiments, A₁ is n-hexyl. In other embodiments, A₁ is n-pentyl. In other embodiments, n-butyl. In still other embodiments, A₁ is n-propyl. In other embodiments, A₁ is ethyl. In yet other embodiments, A₁ is methyl.

In some embodiments, A₂ is absent. In other embodiments, A₂ is n-octyl. In other embodiments, A₂ is n-heptyl. In other embodiments, A₂ is n-hexyl. In some embodiments, A₂ is a C₁₋₅ alkylene. In some embodiments, A₂ is n-pentyl. In other embodiments, A₂ is n-butyl. In still other embodiments, A₂ is n-propyl. In other embodiments, A₂ is ethyl. In still other embodiments, A₂ is methyl.

In some embodiments, X₁ is O. In other embodiments, X₁ is CH₂. In still other embodiments, X₁ is C═O. In some embodiments, X₂ is O. In other embodiments, X₂ is C═O.

In some embodiments, R′ is hydrogen. In other embodiments, R′ is methyl. In some embodiments, R″ is hydrogen. In other embodiments, R″ is methyl. In some embodiments, R′ and R″ taken together with the carbon to which they are attached, is C═O, with the provision that only one of X₁ or R′ and R″ taken together with the carbon may form C═O.

The compounds of the present invention include a selectivity enhancing moiety. The term “selectivity enhancing moiety (SEM)” is defined in U.S. application Ser. No. 11/349,069 filed on Feb. 6, 2006 which is assigned to the assignee of the present application, the contents of which are incorporated herein by reference, refers to one or more moieties that provide an enhancement in the selectivity of the compound to which they are attached for the S1P-1 receptor, as compared to the compound not containing the moiety or moieties. The SEM confers selectivity to the compound to which it is attached for the S1P-1 receptor as compared to, for example, the S1P-2 to S1P-5 receptors. The enhancement conferred to a compound by the SEM may be measured by, for example, determining the binding specificity of a compound for the S1P-1 receptor and one or more of the other S1P receptors wherein enhancement conferred to a compound by the SEM may be in the form of increased potency. In some embodiments, at least one of R₂ and/or R₃ is an SEM. In some embodiments, the SEM is a halo-substituted alkyl group such as CF₃, CF₂CF₃, CF₂CF₂CF₃, CFHCF₃, CH₂CF₃, CH₂CH₂CF₃, CHCl₂, or CH₂Cl.

In certain embodiments, the SEM may possess a selectivity enhancing orientation (SEO). The term “selectivity enhancing orientation” or “SEO,” is defined in U.S. application Ser. No. 11/349,069 filed on Feb. 6, 2006 which is assigned to the assignee of the present application, the contents of which are incorporated herein by reference and as used herein refers to the relative selectivity enhancement of a compound based on the orientation of the SEM as well as the additional substituents on the ring, either alone or in combination with each other. In particular, the SEO may result from the orientation of the SEM on the ring to which it is attached, in relation to any other ring and/or moiety attached to the same ring. In one embodiment, the SEM on 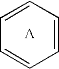
is in the ortho position relative to X₁ in Formula I. In another specific embodiment, the SEM is in the meta position relative to X₁.

Thus, in some embodiments, R₂ is alkyl substituted with 1, 2 or 3 halo groups. In some embodiments, R₂ is trifluoromethyl. In still other embodiments, R₂ is methyl.

In some embodiments, R₃ is absent. For example, in the case of compounds where 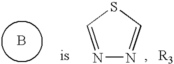
would be considered absent, because there no substituents on the ring. In other embodiments, R₃ is halogen.

In some embodiments, R₄ is hydrogen. In other embodiments, R₄ is an alkyl, e.g., a C₁₋₄ alkyl. For example, in some embodiments, R₄ is methyl. In some embodiments, R₄ is hydroxymethyl.

In some embodiments, R₅ is hydrogen. In some embodiments, R₆ is hydrogen. In some embodiments, R₅ is an alkyl, e.g., a C₁₋₄ alkyl. In some embodiments, R₆ is an alkyl, e.g., a C₁₋₄ alkyl.

In some embodiments, R₇ is OH. In other embodiments, R₇ is CO₂H. In still other embodiments, R₇ is CO₂Me or CO₂Et. In other embodiments, R₇ is CO₂-phenyl. In still other embodiments, R₇ is —OP(O)₃H₂. In other embodiments, R₇ is —CH₂P(O)₃H₂.

In some embodiments, 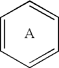
is phenyl. In other embodiments, 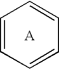
is pyridyl.

In some embodiments, 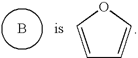
In some embodiments, 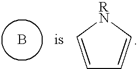
In some embodiments, 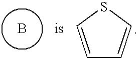
In some embodiments, 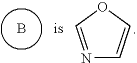
In some embodiments, 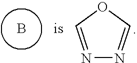
In some embodiments, 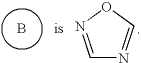
In some embodiments, 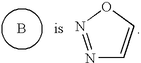
In some embodiments, 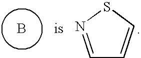
In some embodiments, 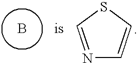
In some embodiments, 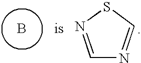
In some embodiments, 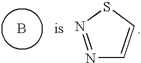
In some embodiments, 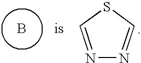
In some embodiments, 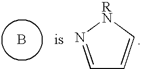
In some embodiments, 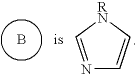
In some embodiments, 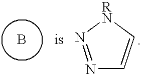
In some embodiments, 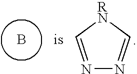
In some embodiments, 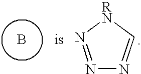
In some embodiments, 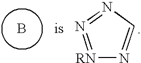
In each of the above structures, R can be hydrogen or alkyl.

In some embodiments, compounds of the invention are compounds wherein

R₁ is hydrogen, aryl, cycloalkyl, or heteroaryl.

R₄ is hydrogen, alkyl, alkylene-OH, aryl, -alkylene-O-alkyl, alkylene-CO₂H, or -alkylene-CO₂-alkyl;

R₅ and R₆ are each independently hydrogen or alkyl, or alkylene-OH;

R₇ is selected from the group consisting of OH, alkylene-OH, —CO₂H, alkylene-CO₂H, -alkylene-CO₂-alkyl, C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, or —C(Z′)(Z″)PO₃Rₚ₁Rₚ₂.

In some embodiments, compounds of the invention are compounds wherein

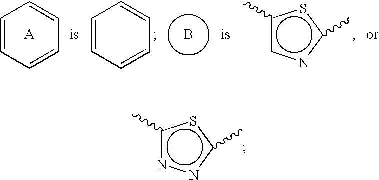

R₁ is hydrogen or aryl, optionally substituted with 1 or 2 groups selected from —CF₃, —CN, —OMe, —Cl or —F;

R₂ is —CF₃;

R₃ is absent or hydrogen;

R₄ is a C₁₋₄ alkyl;

R₅ and R₆ are each independently hydrogen;

R₇ is —OH or —OPO₃Rₚ₁Rₚ₂;

A₁ is (C₁-C₅)alkyl;

A₂ is absent or (C₁-C₅)alkyl;

R′ and R″ are hydrogen;

X₁ is O; and

X₂ is O.

In other embodiments, compounds of the invention are compounds wherein

R₁ is hydrogen or aryl;

R₄ is hydrogen or alkyl;

R₅ and R₆ are each independently hydrogen or alkyl, or alkylene-OH;

R₇ is selected from the group consisting of —OH, alkylene-OH, —CO₂H, alkylene-CO₂H, -alkylene-CO₂-ally, C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂.

In other embodiments, compounds of the invention are compounds wherein

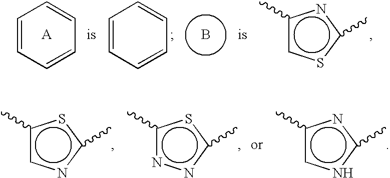

R₁ is phenyl;

A₁ is (C₁-C₈)alkyl;

A₂ is (C₁-C₈)alkyl;

R′ and R″ are hydrogen;

X₁ is O;

X₂ is O;

R₄ is hydrogen, alkyl, or alkylene-OH;

R₅ and R₆ are each independently hydrogen, alkyl;

R₇ is selected from the group consisting of —OH, alkylene-OH, —CO₂H, alkylene-CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂.

In some embodiments, compounds of the invention are compounds of formula I-1.

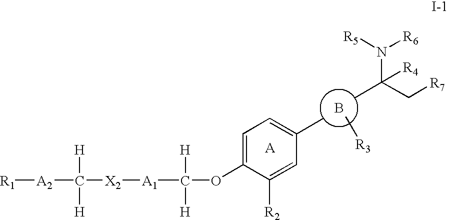

In some embodiments, compounds of the invention are compounds of formula I-2.

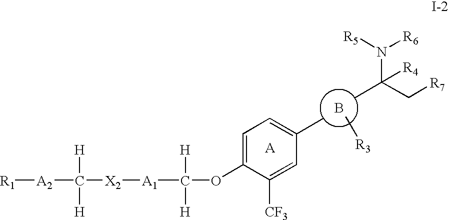

In some embodiments, compounds of formula I are compounds of formula I-3.

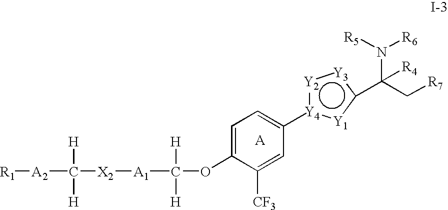

wherein 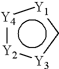
is a heteroaryl ring containing up to three heteroatoms selected from N, O, or S, optionally substituted on carbon with halogen or alkyl, wherein

Y₁ is C, N, S, or O;

Y₂ and Y₃ are each independently C, N, O, or S; provided that when 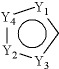
contains an N—H, that hydrogen may be replaced with alkyl; and

Y₄ is C or N.

In some embodiments, compounds of formula I are compounds of formula I-4.

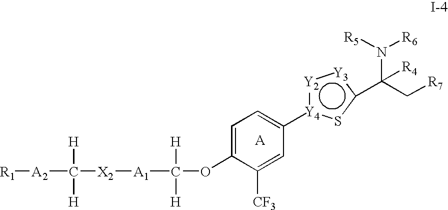

In some embodiments, compounds of formula I are compounds of formula I-5.

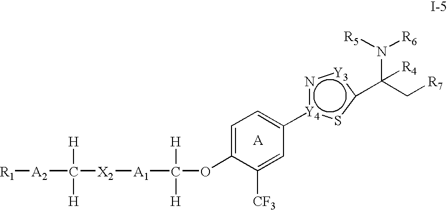

In some embodiments, compounds of formula I are compounds of formula I-6.

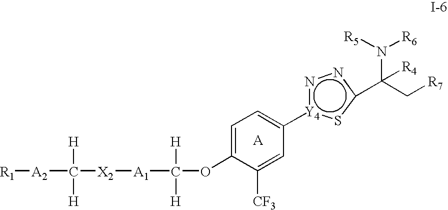

In some aspects, the present invention is directed to a compound of formula II.

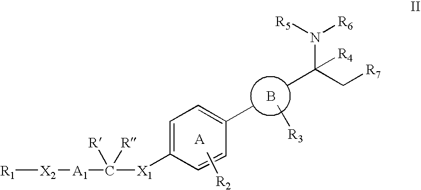

wherein:

R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from O NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 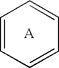
is phenyl or pyridyl; 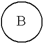
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;

R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein

Z′ is hydroxyl or halogen;

Z″ is H or halogen;

Rₚ₁ and Rₚ₂ are each independently hydrogen, C₁-C₆-alkyl, aryl, or one of the following groups:

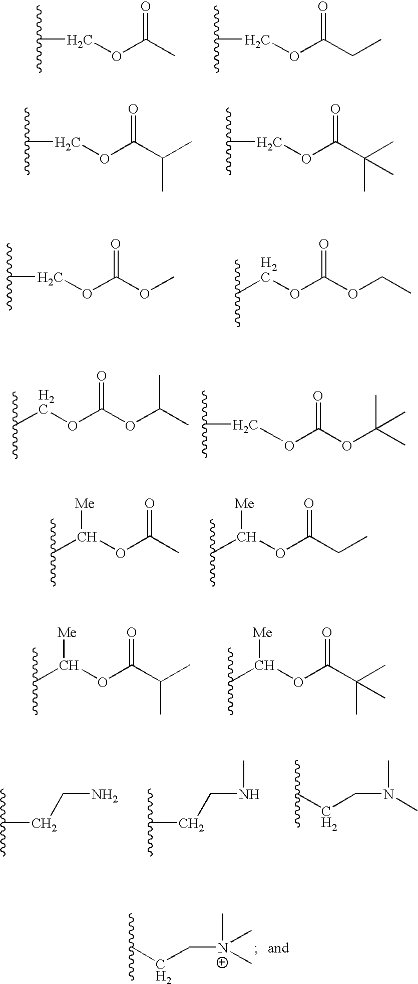

Y is heterocyclo or heteroaryl.

In some embodiments, R₁ is aryl, optionally substituted with 1, 2, or 3 groups selected from halo, alkyl, haloalkyl, —CF₃, —CN, —OH, or —O-alkyl. In some embodiments, R₁ is aryl, e.g., phenyl, optionally substituted with 1 or 2 groups selected from —CF₃, —CN, —OMe, —Cl or —F. In some embodiments, R₁ is phenyl. In other embodiments, R₁ is pyridyl. In still other embodiments, R₁ is thiophenyl. In other embodiments, R₁ is cyclohexyl. In still other embodiments, R₁ is cyclopentyl.

In some embodiments, A₁ is n-octyl. In other embodiments, A₁ is n-heptyl. In still other embodiments, A₁ is n-hexyl. In some embodiments, A₁ is a C₁₋₅ alkylene. In other embodiments, A₁ is n-pentyl. In still other embodiments, A₁ is n-butyl. In other embodiments, A₁ is n-propyl. In yet other embodiments, A₁ is ethyl. In other embodiments, A₁ is methyl.

In some embodiments, X₁ is O. In other embodiments, X₁ is CH₂. In still other embodiments, X₁ is C═O. In some embodiments, X₂ is O. In other embodiments, X₂ is C═O.

In some embodiments, R′ is hydrogen. In other embodiments, R′ is methyl. In some embodiments, R″ is hydrogen. In other embodiments, R″ is methyl. In some embodiments, R′ and R″ taken together with the carbon to which they are attached, is C═O, with the provision that only one of X₁ or R′ and R″ taken together with the carbon may form C═O.

In some embodiments, R₂ is trifluoromethyl. In some embodiments, R₂ is cyano. In other embodiments, R₂ is methyl.

A specific value for R₃ is halogen. For example, in the case of compounds where 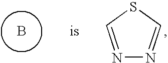
R₃ would be considered absent, because there no substituents on the ring. In other embodiments, R₃ is halogen.

In some embodiments, R₄ is hydrogen. In other embodiments, R₄ is an alkyl, e.g., a C₁₋₄ alkyl. For example, in some embodiments, R₄ is methyl. In some embodiments, R₄ is hydroxymethyl.

In some embodiments, R₅ is hydrogen. In some embodiments, R₆ is hydrogen. In some embodiments, R₅ is an alkyl, e.g., a C₁₋₄ alkyl. In some embodiments, R₆ is an alkyl, e.g., a C₁₋₄ alkyl.

In some embodiments, R₇ is OH. In other embodiments, R₇ is CO₂H. In some embodiments, R₇ is CO₂Me or CO₂Et. In other embodiments, R₇ is CO₂-phenyl. In some embodiments, R₇ is —OP(O)₃H₂. In other embodiments, R₇ is —CH₂P(O)₃H₂.

In some embodiments, 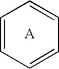
is phenyl. In other embodiments, 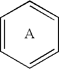
is pyridyl.

In some embodiments, 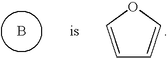
In some embodiments, 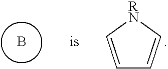
In some embodiments, 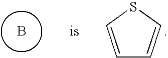
In some embodiments, 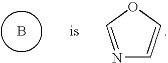
In some embodiments, 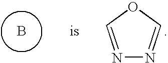
In some embodiments, 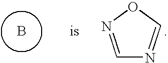
In some embodiments, 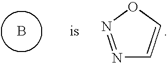
In some embodiments, 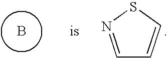
In some embodiments, 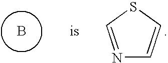
In some embodiments, 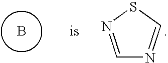
In some embodiments, 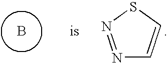
In some embodiments, 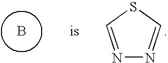
In some embodiments, 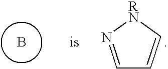
In some embodiments, 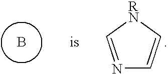
In some embodiments, 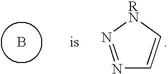
In some embodiments, 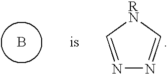
In some embodiments, 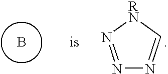
In some embodiments, 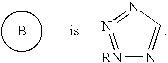
In each of the above structures, R can be hydrogen or alkyl.

In some embodiments, compounds of the invention are compounds wherein

R₁ is hydrogen, aryl, cycloalkyl, or heteroaryl.

R₄ is hydrogen, alkyl, alkylene-OH, aryl, -alkylene-O-alkyl, alkylene-CO₂H, or -alkylene-CO₂-alkyl;

R₅ and R₆ are each independently hydrogen or alkyl, or alkylene-OH;

R₇ is selected from the group consisting of OH, alkylene-OH, —CO₂H, alkylene-CO₂H, -alkylene-CO₂-alkyl, C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, or —C(Z′)(Z″)PO₃Rₚ₁R_{p2.}

In some embodiments, compounds of the invention are compounds wherein

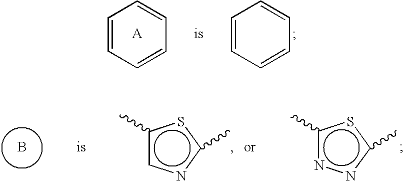

R₁ is hydrogen or aryl, optionally substituted with 1 or 2 groups selected from —CF₃, —CN, —OMe, —Cl or —F;

R₂ is —CF₃;

R₃ is absent or hydrogen;

R₄ is a C₁₋₄ alkyl;

R₅ and R₆ are each independently hydrogen;

R₇ is —OH or —OPO₃Rₚ₁Rₚ₂;

A₁ is (C₁-C₈)alkyl;

R′ and R″ are hydrogen;

X₁ is O; and

X₂ is O.

In other embodiments, compounds of the invention are compounds wherein

R₁ is hydrogen or aryl;

R₄ is hydrogen or alkyl;

R₅ and R₆ are each independently hydrogen or alkyl, or alkylene-OH;

R₇ is selected from the group consisting of —OH, alkylene-OH, —CO₂H, alkylene-CO₂H, -alkylene-CO₂-ally, C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁R_{p2.}

In other embodiments, compounds of the invention are compounds wherein

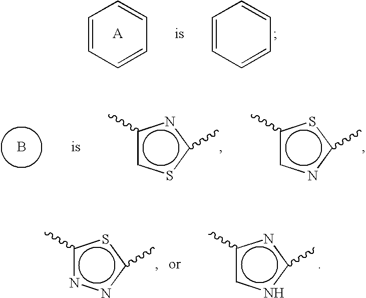

R₁ is phenyl;

A₁ is (C₁-C₈)alkyl;

R′ and R″ are hydrogen;

X₁ is O;

X₂ is O;

R₄ is hydrogen, alkyl, or alkylene-OH;

R₅ and R₆ are each independently hydrogen, alkyl;

R₇ is selected from the group consisting of —OH, alkylene-OH, —CO₂H, alkylene-CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂.

In some embodiments, compounds of the invention are compounds of formula II-1.

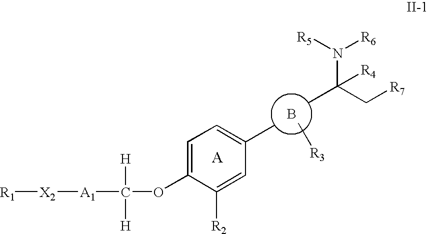

In some embodiments, compounds of the invention are compounds of formula II-2.

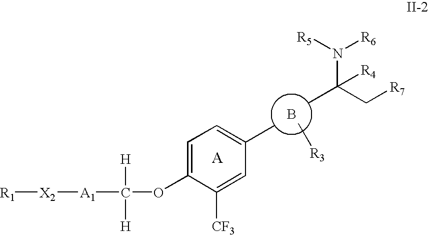

In other embodiments, compounds of formula II are compounds of formula II-3.

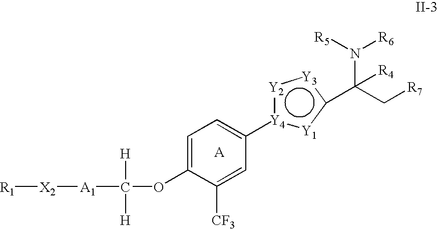

wherein 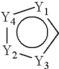
is a heteroaryl ring containing up to three heteroatoms selected from N, O, or S, optionally substituted on carbon with halogen or alkyl, wherein

Y₁ is C, N, S, or O;

Y₂ and Y₃ are each independently C, N, O, or S; provided that when 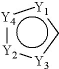
contains an N—H, that hydrogen may be replaced with alkyl; and

Y₄ is C or N.

In other embodiments, compounds of formula II are compounds of formula II-4.

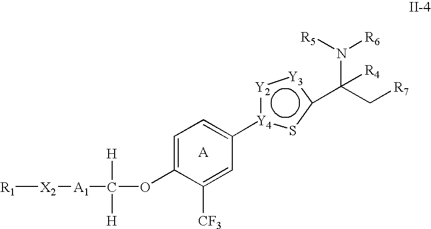

In some embodiments, compounds of formula II are compounds of formula II-5.

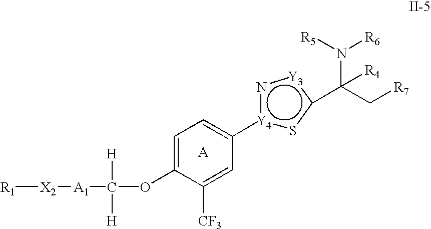

In other embodiments, compounds of formula II are compounds of formula II-6.

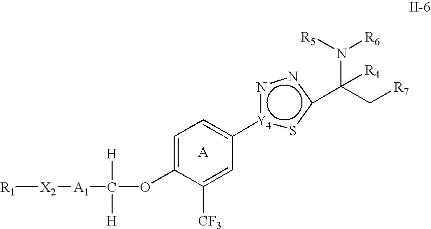

In some embodiments, compounds of the present invention include compounds listed in the following table:

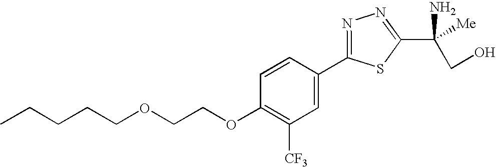

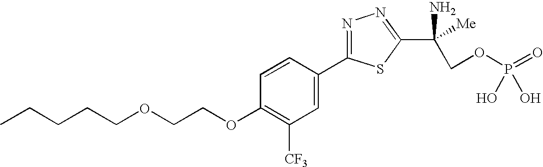

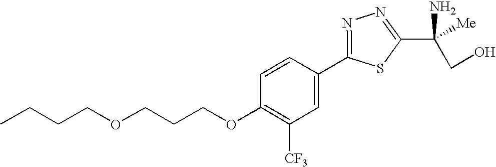

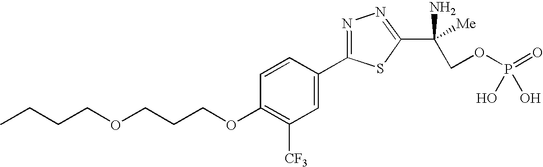

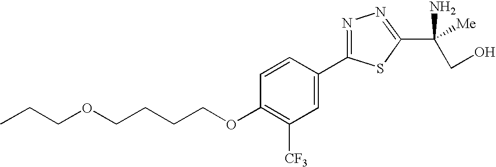

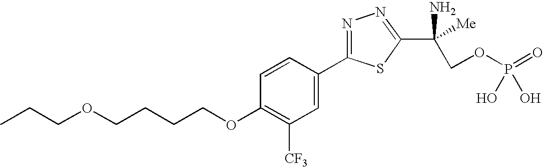

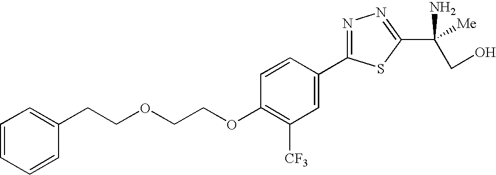

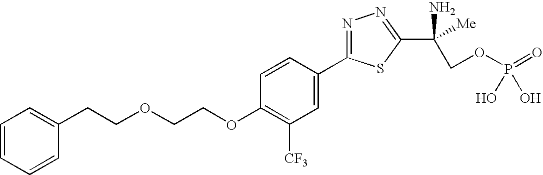

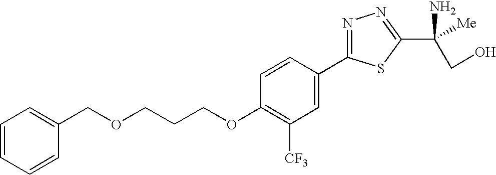

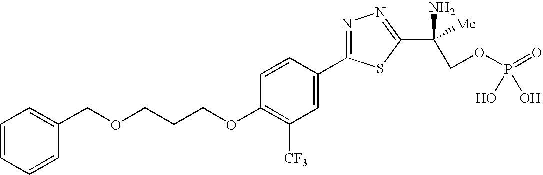

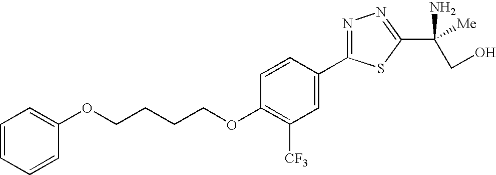

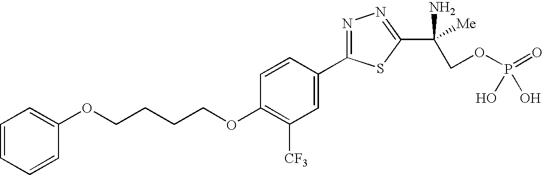

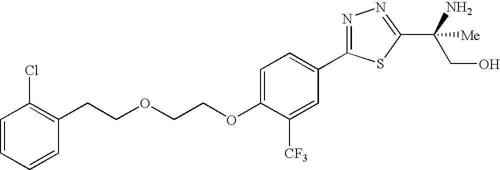

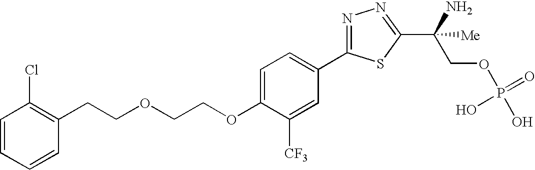

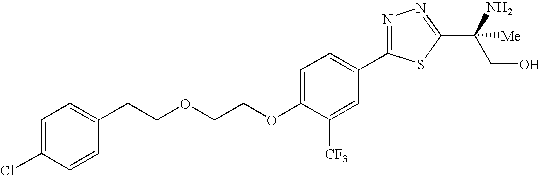

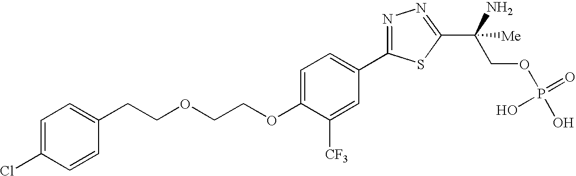

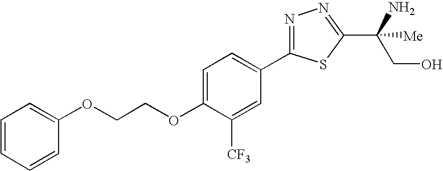

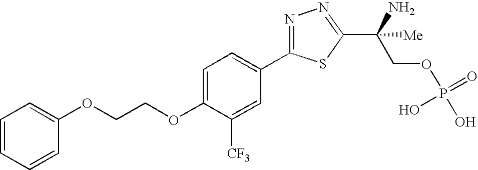

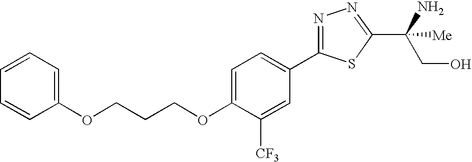

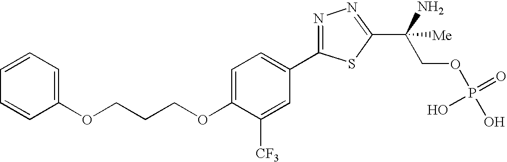

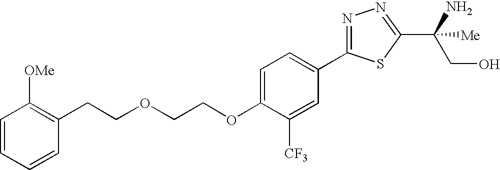

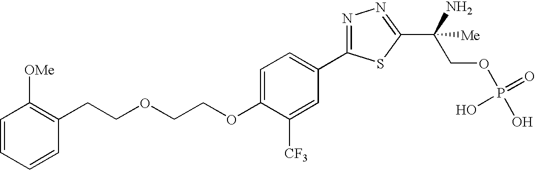

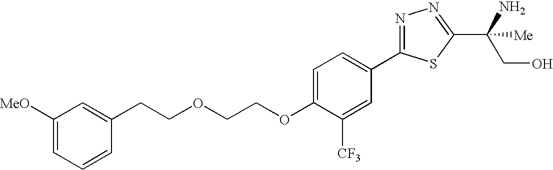

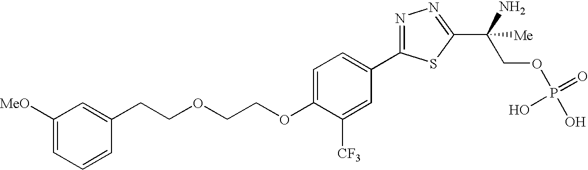

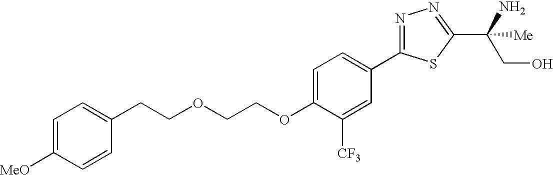

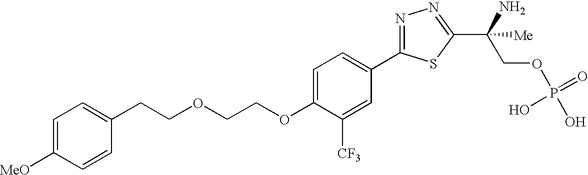

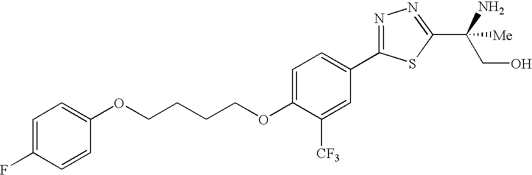

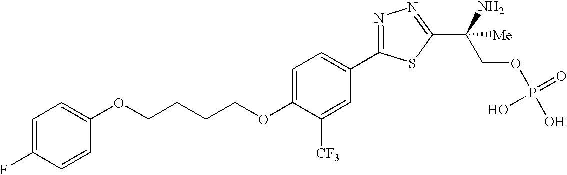

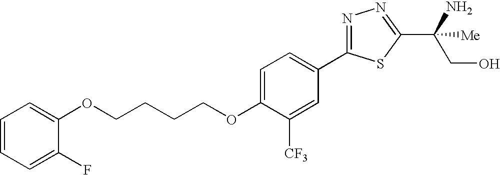

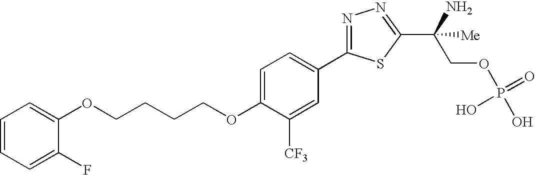

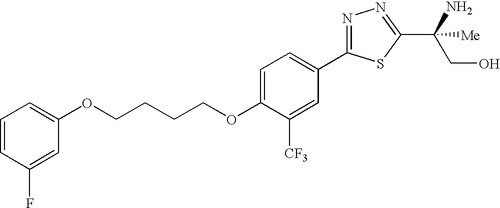

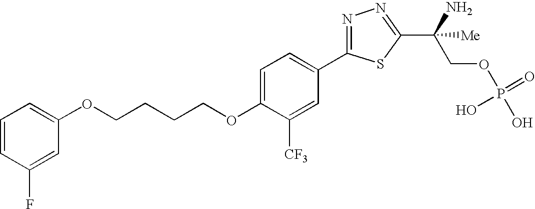

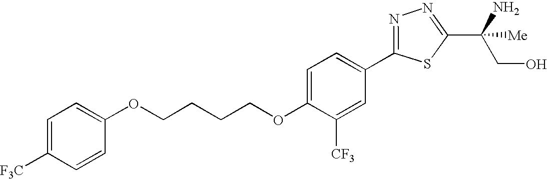

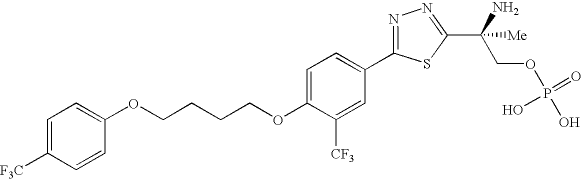

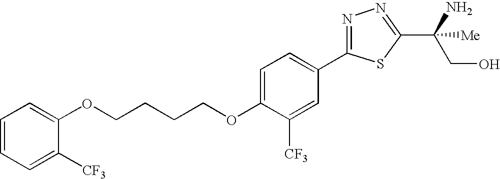

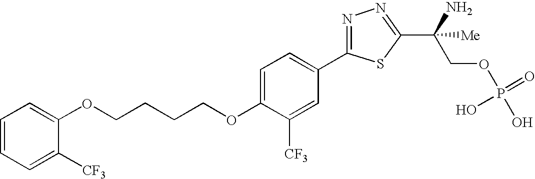

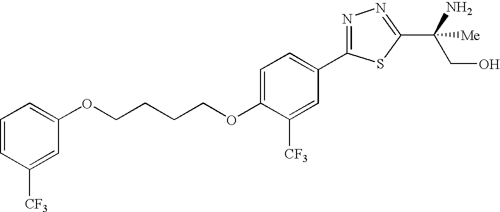

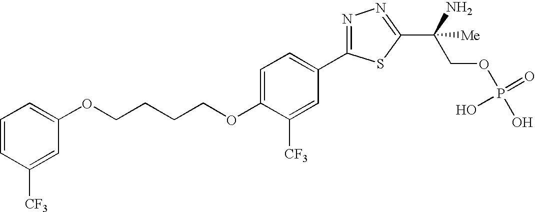

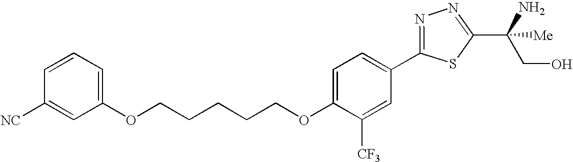

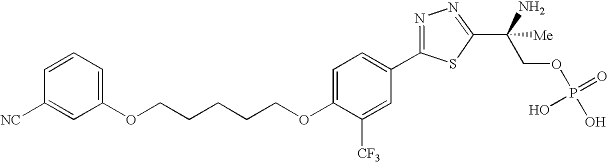

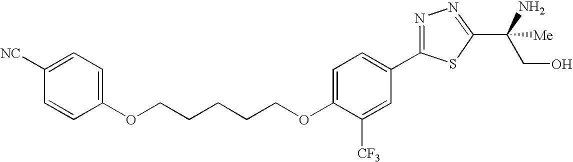

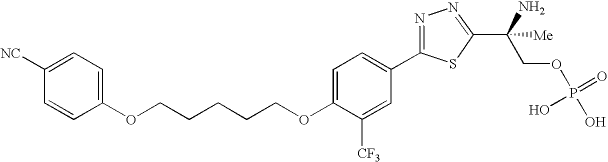

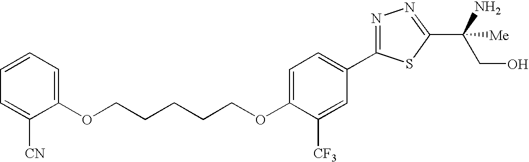

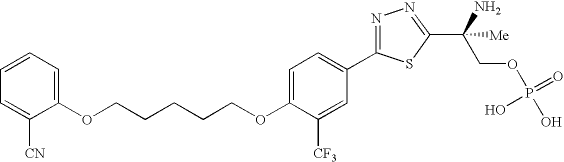

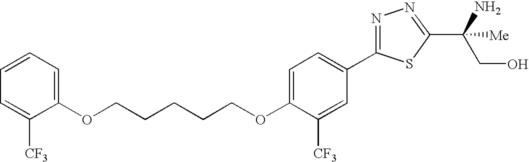

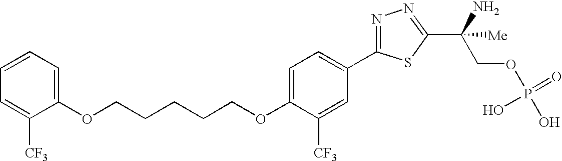

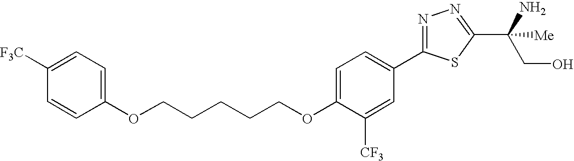

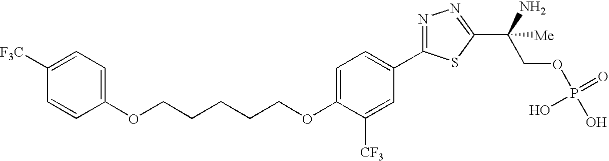

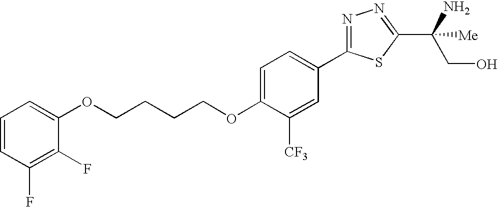

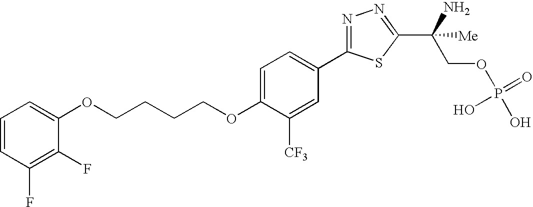

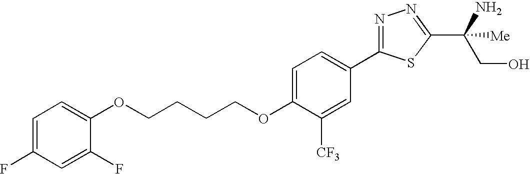

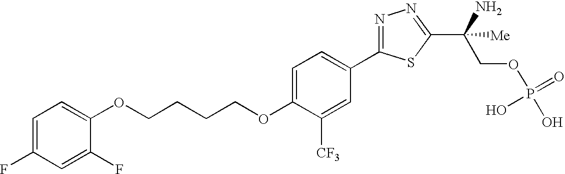

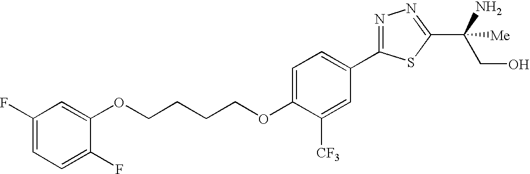

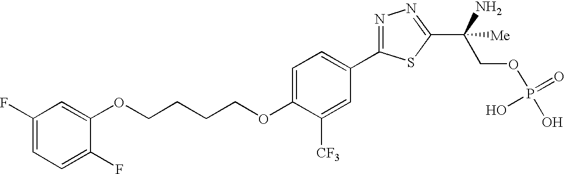

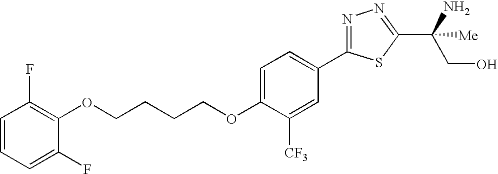

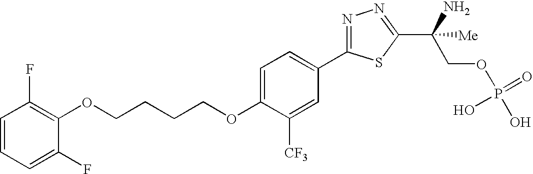

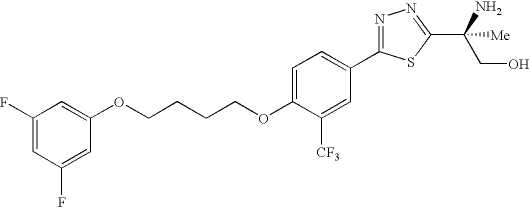

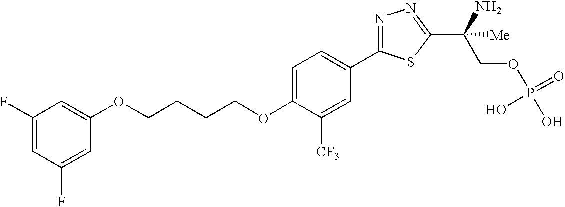

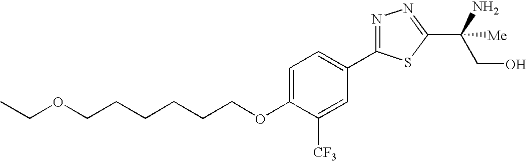

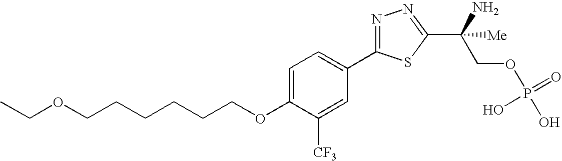

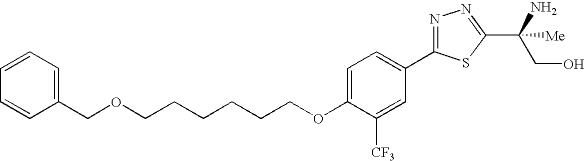

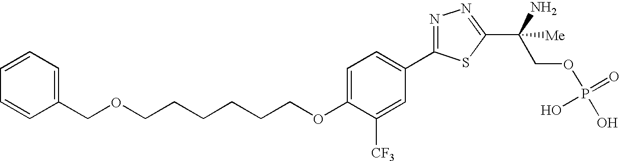

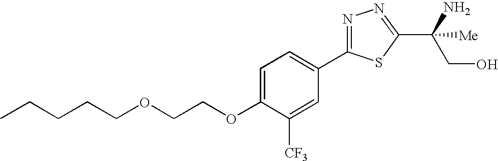

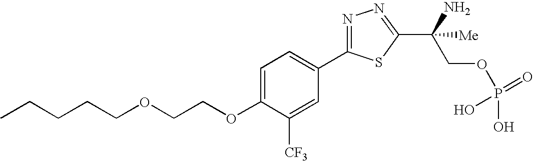

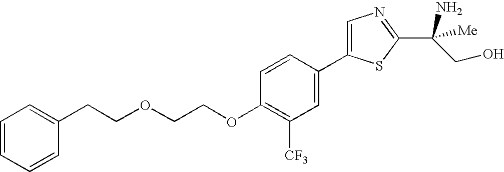

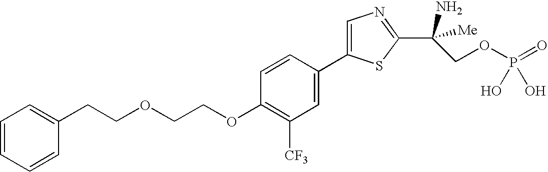

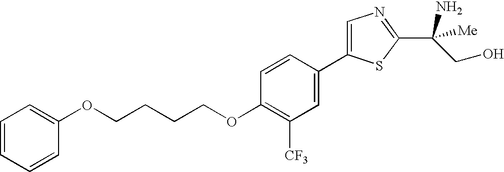

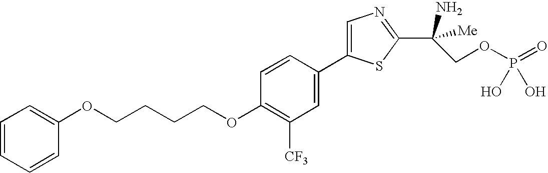

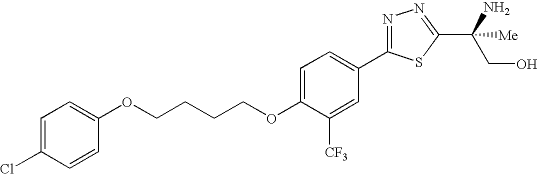

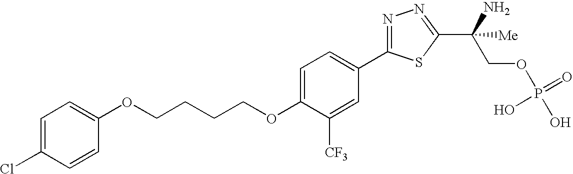

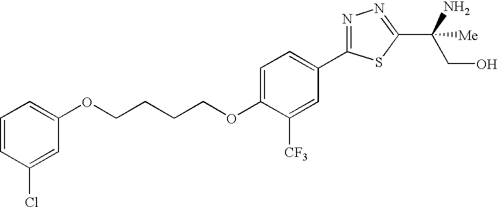

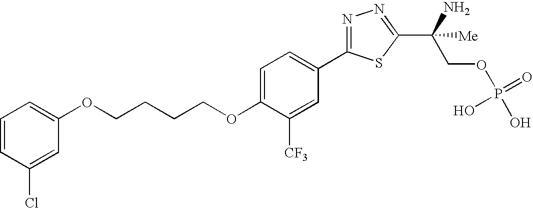

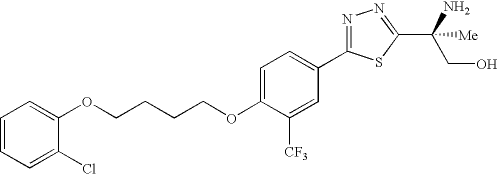

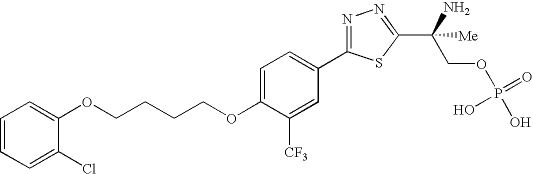

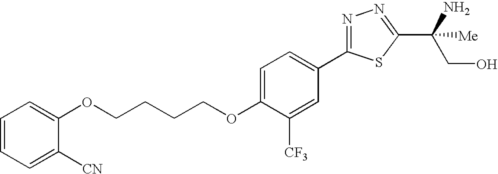

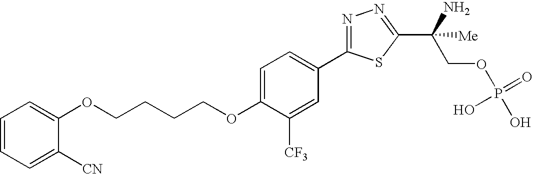

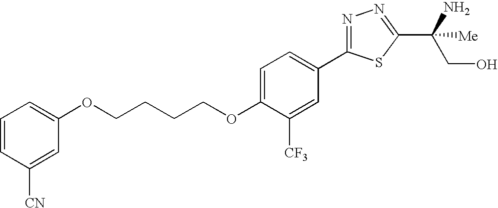

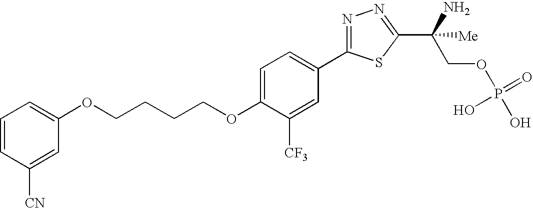

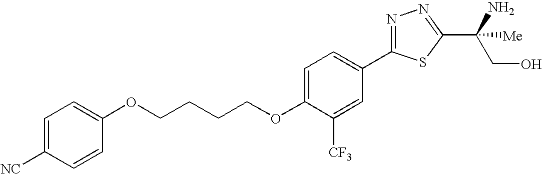

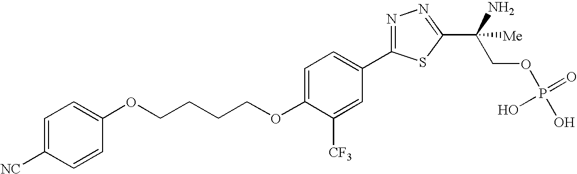

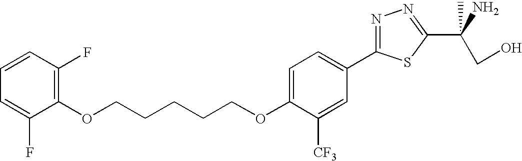

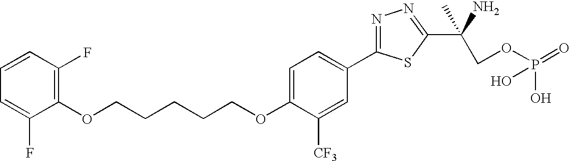

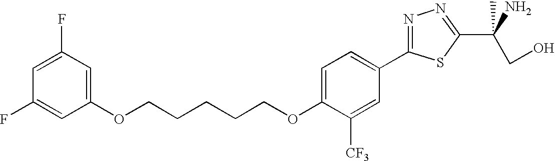

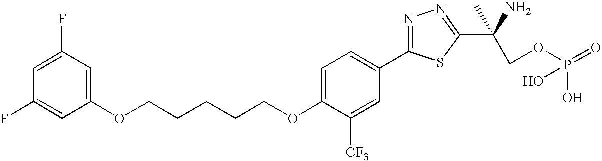

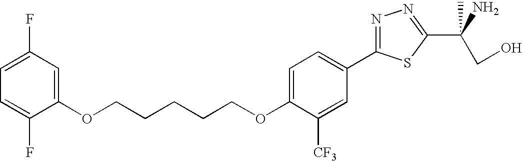

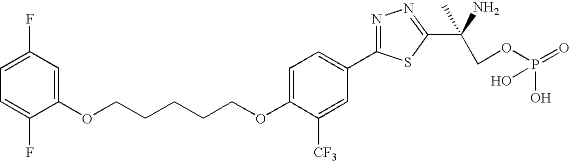

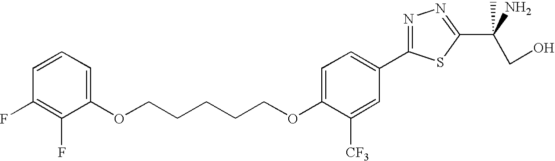

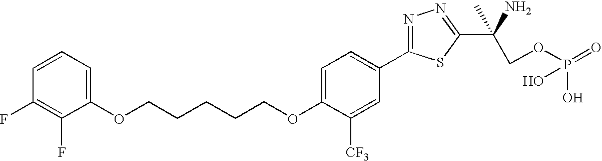

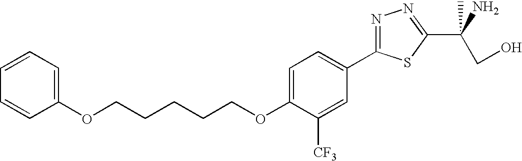

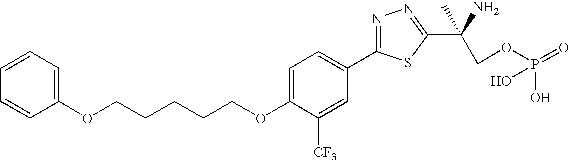

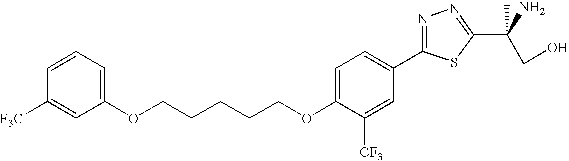

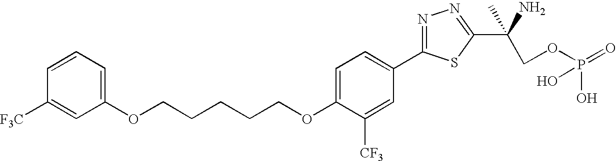

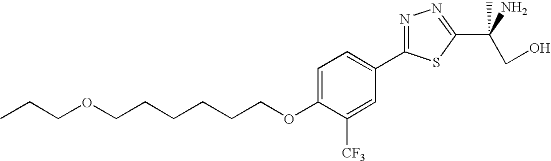

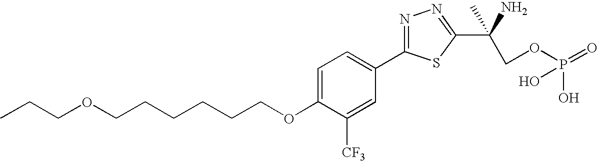

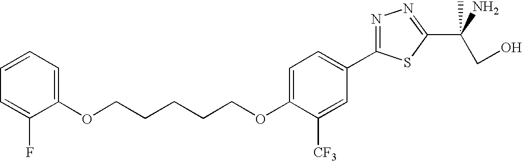

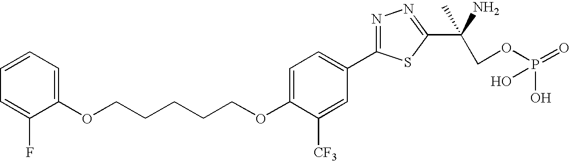

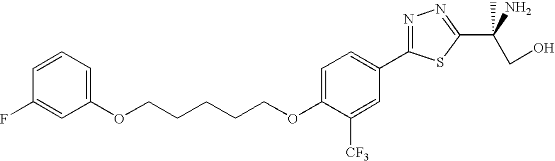

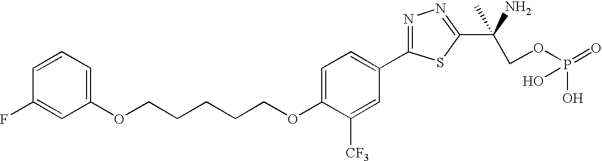

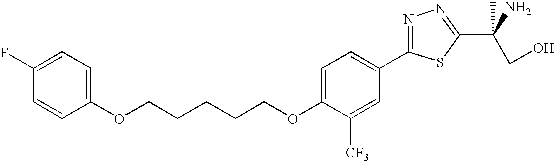

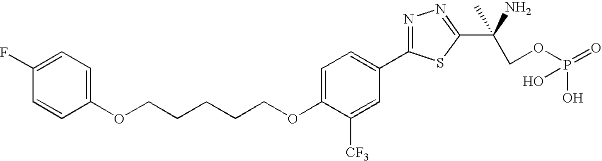

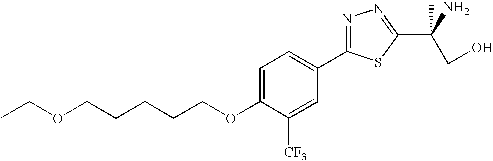

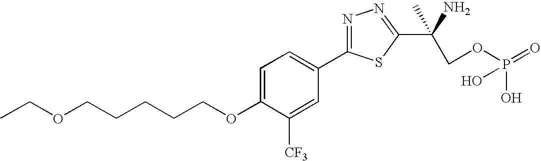

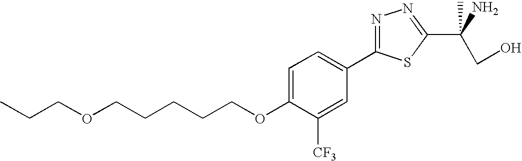

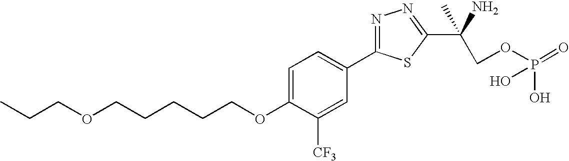

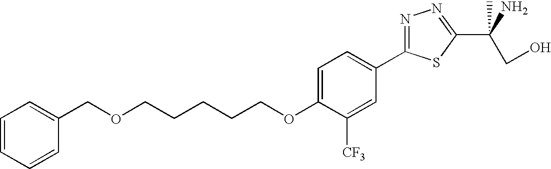

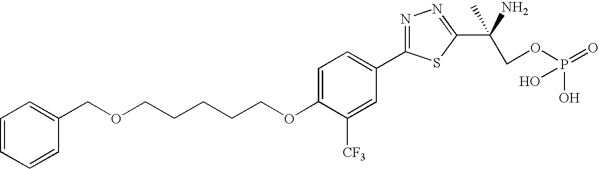

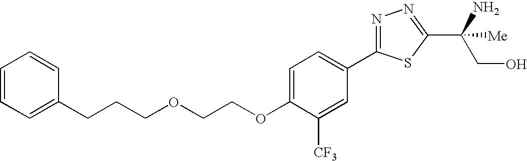

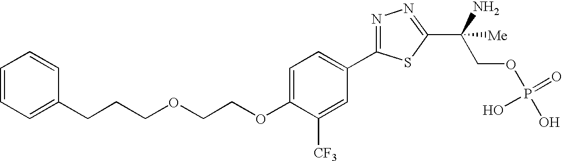

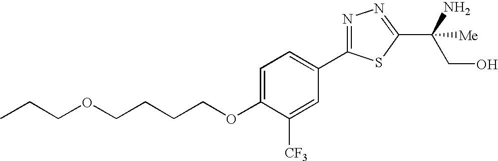

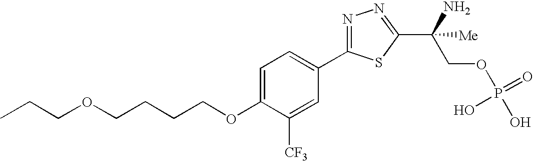

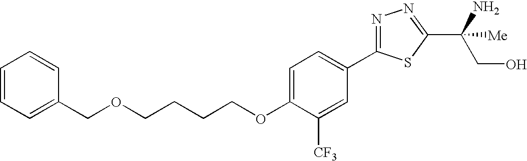

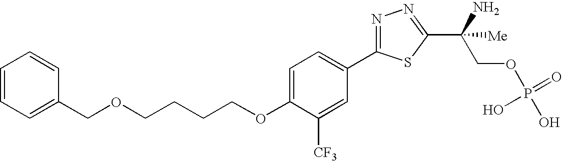

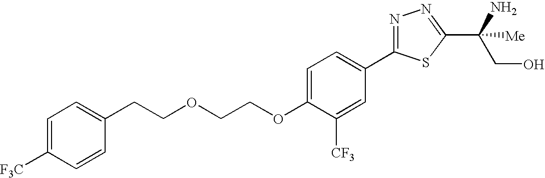

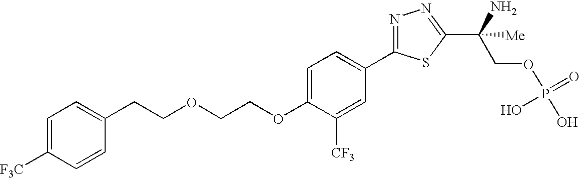

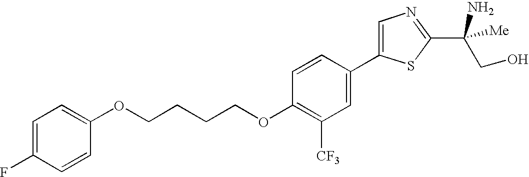

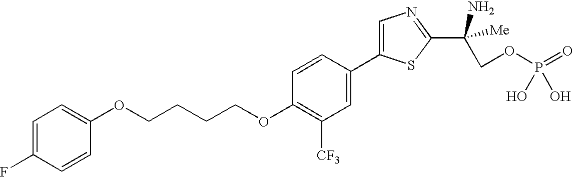

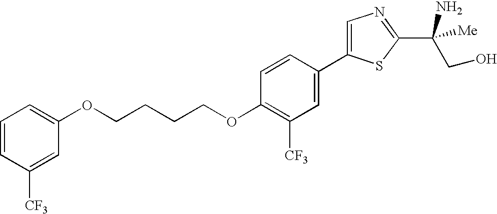

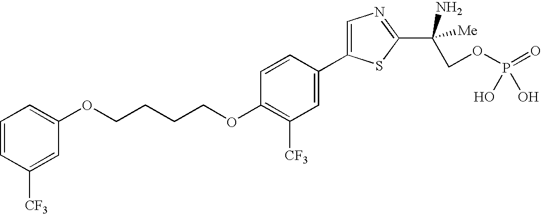

and pharmaceutically acceptable salts, phosphate derivatives, phosphate mimics, or phosphate precursor analogs thereof.

In some aspects, the present invention is directed to a compound of formula III

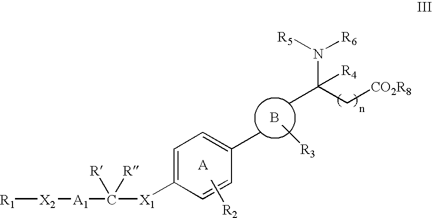

or a pharmaceutically acceptable salt thereof, wherein:

R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;

A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, CH₂O, S, —S(O), —S(O)₂, —C(O)—, —C(O)O—, or NRₓ, wherein Rₓ is H or (C₁-C₆)alkyl;

X₂ is O, CH₂O, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen

R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 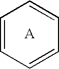
is phenyl or pyridyl; 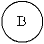
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;

R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

n is 0, 1, or 2;

R₈ is hydrogen, alkyl, or aryl.

In some embodiments, individual values for R₁, R′, R″, X₁, X₂, 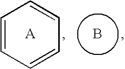
R₃, R₄, R₅, R₆; and R₇ are as provided for a compound of formula I.

In some embodiments, n is 0. In other embodiments, n is 1. In still other embodiments, n is 2.

In some embodiments, R₈ is hydrogen. In some embodiments, R₈ is a C₁₋₄ alkyl. In some embodiments, R₈ is methyl. In some embodiments, R₈ is ethyl. In some embodiments, R₈ is phenyl.

In some aspects, the present invention is directed to the compounds of the following table:

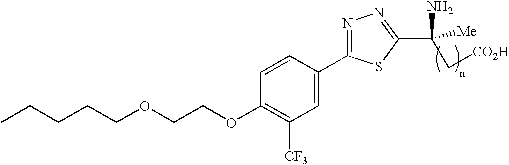

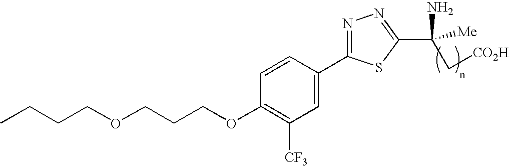

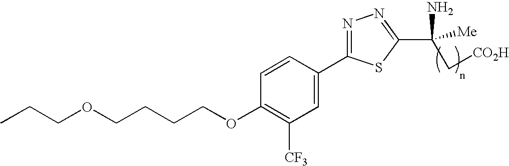

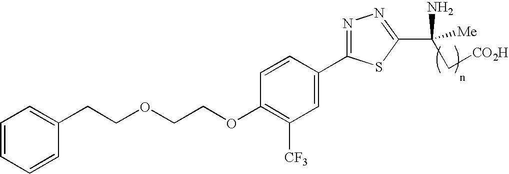

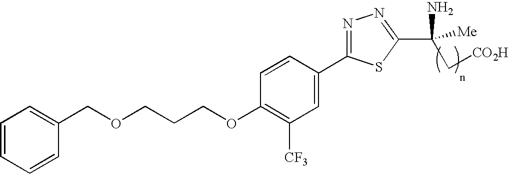

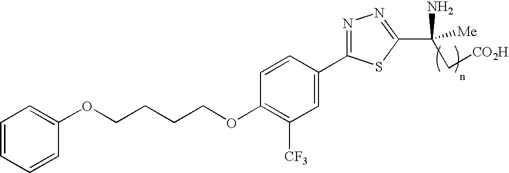

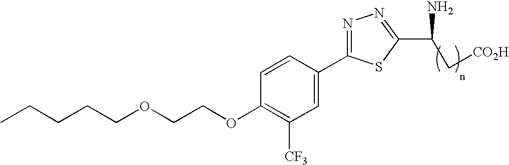

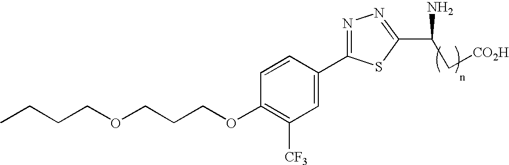

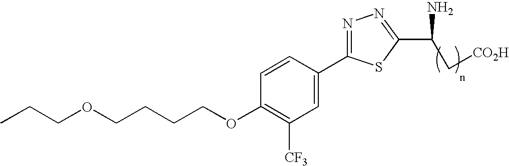

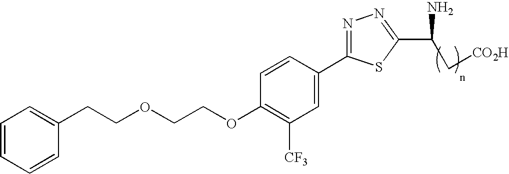

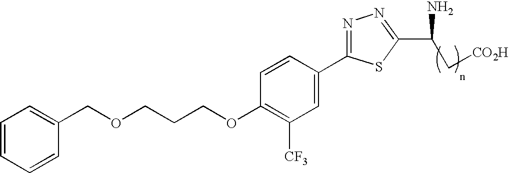

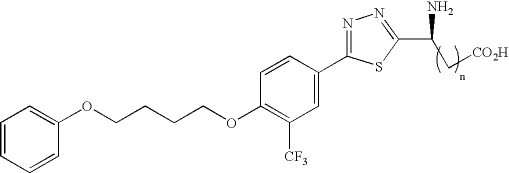

wherein n for each compound is 0, 1, or 2, as well as pharmaceutically acceptable salts, phosphate derivatives, phosphate mimics, or phosphate precursor analogs thereof.

In some embodiments, compounds of the present invention do not include the compounds listed in WO 05/041899, WO 04/010949, WO 04/02463, WO 06/020951, and U.S. Ser. No. 11/349,069, the latter two of which are assigned to the same assignee as the present application.

Biological Activity of Compounds of the Invention

Lymphopenia Assay

Several of the compounds described herein were evaluated for the ability to induce lymphopenia in mice. Male C57B1/6 mice were divided into groups of three. A control group received the 3% BSA vehicle only. The other groups received a single dose of either a specified dose of test compound in vehicle administered orally (PO) and intravenously (IV). After 6 hours, the mice were anesthesized with isoflurane and approximately 250 μL of blood was removed from the retroorbital sinus and collected in an EDTA microtainer, mixed with an anticoagulant and placed on a tilt table until complete blood count (CBC) analysis. Oral administration (10 mg/K) of these compounds induced increased lymphopenia versus the vehicle.

Binding to S1P-1 or S1P-3 Receptors

In certain embodiments, the compounds of the invention selective for the S1P-1 receptor as compared to one or more of the other S1P receptors. For example, one set of compounds includes compounds which are selective for the S1P-1 receptor relative to the S1P-3 receptor. Compounds selective for the S1P-1 receptor can be agonists of the S1P-1 receptor, significantly weaker agonists of one or more other receptors and/or antagonists of one or more other receptors. A compound is “selective” for the S1P-1 receptor relative to a second receptor, if the EC₅₀ of the compound for the second receptor is at least two-fold greater than the EC₅₀ for the S1P-1 receptor. The EC₅₀ of a compound is determined using the ³⁵S-GTPγS binding assay, as described in WO 03/061567, the entire contents of which are incorporated herein by reference. Additionally or alternatively, a compound is “selective” for the S1P-1 receptor relative to a second receptor, if the IC₅₀ of the compound for the second receptor is at least two-fold greater than the IC₅₀ for the S1P-1 receptor. The IC₅₀ of a compound is determined using the [³³P]sphingosine 1-phosphate binding assay, as described in Davis, M. D. et al., Sphingosine 1-Phosphate Analogs as Receptor Antagonists. J. Biol. Chem. (2005) 280:9833-9841, the entire contents of which are incorporated herein by this reference.

The terms “agonist” or “S1P-1 receptor agonist” as used herein include the compounds described herein which bind to and/or agonize the S1P-1 receptor. In one embodiment, the S1P receptor agonists have an IC₅₀ for the S1P-1 receptor of about 100 nM-0.25 nM, about 50 nM-0.25 nM, about 25 nM-0.5 nM, about 100 nM or less, about 75 nM or less, about 50 nM or less, about 40 nM or less, about 30 nM or less, about 20 nM or less, about 10 nM or less, about 5 nM or less, about 1 nM or less, about 0.5 nM or less, or about 0.25 nM or less. The compounds' IC₅₀ for the S1P1 receptor can be measured using the binding assays described in Example 13 or those described in WO 03/061567. Compounds of the invention generally had an IC₅₀ in the range of 100 pM (picomolar) to 100 M.

For example, 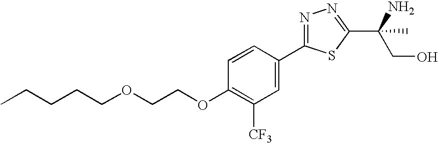
had an IC₅₀ 2.17 nM

Ranges intermediate to the above recited values are also intended to be part of this invention. For example, ranges using a combination of any of the above recited values as upper and/or lower limits are intended to be included.

In a further embodiment, the S1P receptor agonist has an IC₅₀ value for the S1P-3 receptor of about 10 nM-10,000 nM, about 100 nM-5000 nM, about 100 nM-3000 nM, about 10 nM or greater, about 20 nM or greater, about 40 nM or greater, about 50 nM or greater, about 75 nM or greater, or about 100 nM or greater. In another embodiment, the S1P compound of the invention binds the S1P-3 receptor with an IC₅₀ of 1000 nM or greater, 2000 nM or greater, 3000 nM or greater, 5000 nM or greater, 10,000 nM or greater. The IC₅₀ for of S1P-3 receptor can be measured using the binding assays described herein or those described in WO 03/061567.

In addition, it should be understood that the ranges intermediate to the above recited values are also intended to be part of this invention. For example, ranges using a combination of any of the above recited values as upper and/or lower limits are intended to be included.

In yet another embodiment, the S1P receptor agonists described herein have an IC₅₀ value for the S1P-1 receptor that is about 5-fold lower, about 10-fold lower, about 20-fold lower, about 50-fold lower, about 100-fold lower, about 200-fold lower, about 500-fold lower or about 1000-fold lower than their IC₅₀ value for the S1P-3 receptor.

Ranges intermediate to the above recited values are also intended to be part of this invention. For example, ranges using a combination of any of the above recited values as upper and/or lower limits are intended to be included.

The ability of several of the compounds described herein to bind to the S1P-1 or S1P-3 receptor was also tested as follows.

For the membrane preparation, plasmid DNA was transfected into HEK 293 T cells using the FuGENE 6 transfection protocol (publicly available from Roche). Briefly, subconfluent monolayers of HEK 293 T cells were transfected with the DNA mixture containing FuGENE 6 (using a 1:3 ratio). The dishes containing the cells were then placed in a tissue culture incubator (5% CO₂, 37° C.). The cells were harvested 48 hours after addition of the DNA by scraping in HME buffer (in mM: 20 HEPES, 5 MgCl₂, 1 EDTA, pH 7.4, 1 mM PMSF) containing 10% sucrose on ice, and disrupted using a Dounce homogenizer. After centrifugation at 800×g, the supernatant was diluted with HME without sucrose and centrifuged at 17,000×g for 1 hour. This crude membrane pellet was resuspended in HME with sucrose, aliquoted, and snap-frozen by immersion in liquid nitrogen. The membranes were stored at −70 C. Protein concentration was determined spectroscopically by Bradford protein assay.

For the binding assay, [³³P]sphingosine 1-phosphate (obtained from American Radiolabeled Chemicals, Inc) was added to membranes in 200 μl in 96-well plates with assay concentrations of 2.5 pM [³³P]sphingosine 1-phosphate, 4 mg/ml BSA, 50 mM HEPES, pH 7.5, 100 mM NaCl, 5 mM MgCl2, and 5 μg of protein. Binding was performed for 60 minutes at room temperature with gentle mixing and terminated by collecting the membranes onto GF/B filter plates. After drying the filter plates for 10 minutes, 50 μl of Microscint 40 was added to each well, and filter-bound radionuclide was measured on a Packard Top Count. Nonspecific binding was defined as the amount of radioactivity remaining in the presence of excess of unlabeled S1P.

Methods of Using Compounds of the Invention

The compounds of the invention have been determined to be useful in the treatment of sphingosine 1-phosphate associated disorders. Accordingly, in one embodiment, the invention relates to a method for treating a subject suffering from a sphingosine 1-phosphate associated disorder, comprising administering to a subject an effective amount of a compound of the invention; that is, a compound of formula I or compounds otherwise described herein, such that the subject is treated for a sphingosine 1-phosphate associated disorder.

The term “sphingosine 1-phosphate associated disorder” includes disorders, diseases or conditions which are associated with or caused by a misregulation in S1P receptor function and/or signaling or S1P receptor ligand function. The term also includes diseases, disorders or conditions which can be treated by administering to a subject an effective amount of a sphingosine 1-phosphate receptor agonist. Such disorders include disorders that are associated with an inappropriate immune response and conditions associated with an overactive immune response, e.g., autoimmune diseases. In some embodiments, sphingosine 1-phosphate associated disorders include autoimmune diseases. In other embodiments, sphingosine 1-phosphate associated disorders include inflammation. In further embodiments, sphingosine 1-phosphate associated disorders include transplant rejection. In still other embodiments, sphingosine 1-phosphate associated disorders include acute respiratory distress syndrome (ARDS). In other embodiments, sphingosine 1-phosphate associated disorders include asthma. In yet other embodiments, sphingosine 1-phosphate associated disorders include any combination of the disorders listed herein.

“Treatment”, or “treating” as used herein, is defined as the application or administration of a therapeutic agent such as a compound of formula I to a subject who has a sphingosine 1-phosphate associated disorder as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically.

In some embodiments, the efficacy of the compounds of the present invention can be measured by comparing a value, level, feature, characteristic, property, etc. to a “suitable control”. A “suitable control” is any control or standard familiar to one of ordinary skill in the art useful for comparison purposes. In one embodiment, a “suitable control” is a value, level, feature, characteristic, property, etc. determined prior to administering a composition of the present invention. For example, the immune response, etc. can be determined prior to introducing a compound of the invention into a cell or subject. In another embodiment, a “suitable control” is a value, level, feature, characteristic, property, etc. determined in a cell or organism, e.g., a control or normal cell or organism, exhibiting, for example, normal traits. In yet another embodiment, a “suitable control” is a predefined value, level, feature, characteristic, property, etc. For example a “suitable control” can be a pre-defined level of binding to a specified S1P receptor.

An additional embodiment of the invention pertains to a method for treating a subject suffering from a sphingosine 1-phosphate associated disorder, comprising administering to a subject a compound, such that the subject is treated for a sphingosine 1-phosphate associated disorder by a compound of the invention; that is, a compound of formulae I or compounds otherwise described herein.

The present invention is also directed to a method of selectively treating a sphingosine 1-phosphate associated disorder, comprising administering to a subject an effective amount of a compound of the invention, e.g., compounds of any of Formulae I-III or compounds otherwise described herein, such that the subject is selectively treated for a sphingosine 1-phosphate associated disorder. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

Another embodiment of the invention is a method of selectively treating a sphingosine 1-phosphate associated disorder, comprising administering to a subject a compound, such that the subject is selectively treated for a sphingosine 1-phosphate associated disorder by a compound of the invention, e.g., compounds of any of Formulae I-VIII or compounds otherwise described herein. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

In another embodiment, the present invention provides a method of treating a condition associated with an activated immune system. Such diseases or disorders include multiple sclerosis as well as rejection of transplanted organs, tissue or cells; graft-versus-host diseases brought about by transplantation; autoimmune syndromes including rheumatoid arthritis; systemic lupus erythematosus; antiphospholipid syndrome; Hashimoto's thyroiditis; lymphocytic thyroiditis; myasthenia gravis; type I diabetes; uveitis; episcleritis; scleritis; Kawasaki's disease, uveo-retinitis; posterior uveitis; uveitis associated with Behcet's disease; uveomeningitis syndrome; allergic encephalomyelitis; chronic allograft vasculopathy; post-infectious autoimmune diseases including rheumatic fever and post-infectious glomerulonephritis; inflammatory and hyperproliferative skin diseases; psoriasis; psoriatic arthritis; atopic dermatitis; myopathy; myositis; osteomyelitis; contact dermatitis; eczematous dermatitis; seborrhoeic dermatitis; lichen planus; pemphigus; bullous pemphigoid; epidermolysis bullosa; urticaria; angioedema; vasculitis; erythema; cutaneous eosinophilia; acne; scleroderma; alopecia greata; keratoconjunctivitis; vernal conjunctivitis; keratitis; herpetic keratitis; dystrophia epithelialis corneas; corneal leukoma; ocular pemphigus; Mooren's ulcer; ulcerative keratitis; scleritis; Graves' opthalmopathy; Vogt-Koyanagi-Harada syndrome; sarcoidosis; pollen allergies; reversible obstructive airway disease; bronchial asthma; allergic asthma; intrinsic asthma; extrinsic asthma; dust asthma; chronic or inveterate asthma; late asthma and airway hyper-responsiveness; bronchiolitis; bronchitis; endometriosis; orchitis; gastric ulcers; ischemic bowel diseases; inflammatory bowel diseases; necrotizing enterocolitis; intestinal lesions associated with thermal burns; coeliac disease; proctitis; eosinophilic gastroenteritis; mastocytosis; Crohn's disease; ulcerative colitis; vascular damage caused by ischemic diseases and thrombosis; atherosclerosis; fatty heart; myocarditis; cardiac infarction; aortitis syndrome; cachexia due to viral disease; vascular thrombosis; migraine; rhinitis; eczema; interstitial nephritis; IgA-induced nephropathy; Goodpasture's syndrome; hemolytic-uremic syndrome; diabetic nephropathy; glomerulosclerosis; glomerulonephritis; tubulointerstitial nephritis; interstitial cystitis; multiple myositis; Guillain-Barre syndrome; Meniere's disease; polyneuritis; multiple neuritis; myelitis; mononeuritis; radiculopathy; hyperthyroidism; Basedow's disease; thyrotoxicosis; pure red cell aplasia; aplastic anemia; hypoplastic anemia; idiopathic thrombocytopenic purpura; autoimmune hemolytic anemia; autoimmune thrombocytopenia; agranulocytosis; pernicious anemia; megaloblastic anemia; anerythroplasia; osteoporosis; fibroid lung; idiopathic interstitial pneumonia; dermatomyositis; leukoderma vulgaris; ichthyosis vulgaris; photoallergic sensitivity; cutaneous T cell lymphoma; polyarteritis nodosa; Huntington's chorea; Sydenham's chorea; myocardosis; myocarditis; scleroderma; Wegener's granuloma; Sjogren's syndrome; adiposis; eosinophilic fascitis; lesions of gingiva, periodontium, alveolar bone, substantia ossea dentis; male pattern alopecia or alopecia senilis; muscular dystrophy; pyoderma; Sezary's syndrome; hypophysitis; chronic adrenal insufficiency; Addison's disease; ischemia-reperfusion injury of organs which occurs upon preservation; endotoxin shock; pseudomembranous colitis; colitis caused by drug or radiation; ischemic acute renal insufficiency; chronic renal insufficiency; lung solid cancer; malignancy of lymphoid origin; acute or chronic lymphocytic leukemias; lymphoma; psoriasis; pulmonary emphysema; cataracts; siderosis; retinitis pigmentosa; senile macular degeneration; vitreal scarring; corneal alkali burn; dermatitis erythema; ballous dermatitis; cement dermatitis; gingivitis; periodontitis; sepsis; pancreatitis; peripheral artery disease; carcinogenesis; solid cancer tumors; metastasis of carcinoma; hypobaropathy; autoimmune hepatitis; primary biliary cirrhosis; sclerosing cholangitis; partial liver resection; acute liver necrosis; cirrhosis; alcoholic cirrhosis; hepatic failure; fulminant hepatic failure; late-onset hepatic failure; “acute-on-chronic” liver failure.

As used herein, the term “subject” includes warm-blooded animals, e.g., mammals, including humans, cats, dogs, horses, bears, lions, tigers, ferrets, rabbits, mice, cows, sheep, pigs, etc. In a particular embodiment, the subject is a primate. In a specific embodiment, the primate is a human.

As used herein, the term “administering” to a subject includes dispensing, delivering or applying a compound of the invention in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, topical delivery, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat the condition in a subject. An effective amount of a compound of the invention, as defined herein, may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of a compound of the invention (i.e., an effective dosage) may range from about 0.001 to 30 mg/kg body weight, for example, about 0.01 to 25 mg/kg body weight, for example, about 0.1 to 20 mg/kg body weight. It is to be understood that all values and ranges between those listed are intended to be encompassed by the present invention. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, for example, can include a series of treatments. It will also be appreciated that the effective dosage of the compound used for treatment may increase or decrease over the course of a particular treatment.

The methods of the invention further include administering to a subject a therapeutically effective amount of a compound of the invention in combination with another pharmaceutically active compound known to treat the disease or condition, e.g., an immunomodulatory agent or an anti-inflammatory agent. Pharmaceutically active compounds that may be used depend upon the condition to be treated, but include as examples cyclosporin, rapamycin, FK506, methotrexate, etanercept, infliximab, adalimumab, non-steroidal anti-inflammatory agents, cyclooxygenase-2-inhibitors, such as celecoxib and rofecoxib, and corticosteroids. Other suitable compounds can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The compound of the invention and the additional pharmaceutically active compound may be administered to the subject in the same pharmaceutical composition or in different pharmaceutical compositions (at the same time or at different times).

Pharmaceutical Compositions Comprising Compounds of the Invention

The present invention also provides pharmaceutically acceptable formulations and compositions comprising one or more compounds of the invention; that is, compounds of formula I or compounds otherwise described herein. In certain embodiments, the compound of the invention is present in the formulation in a therapeutically effective amount; that is, an amount effective to treat a sphingosine 1-phosphate associated disorder.

Accordingly, in one embodiment, the invention pertains to a pharmaceutical composition comprising a therapeutically effective amount of a compound of the invention; that is, compounds of formula I or compounds otherwise described herein, and a pharmaceutically acceptable carrier.

In another embodiment, the invention is directed to a packaged pharmaceutical composition comprising a container holding a therapeutically effective amount of a compound of the invention; that is, compounds of formula I or compounds otherwise described herein; and instructions for using the compound to treat a sphingosine 1-phosphate associated disorder in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing a sphingosine 1-phosphate associated disorder in a subject.

Another embodiment of the invention relates to a packaged pharmaceutical composition comprising a container holding a therapeutically effective amount of a compound of the invention; that is, a compound of formula I or compounds otherwise described herein, and instructions for using the compound to selectively treat a sphingosine 1-phosphate associated disorder in a subject.

Such pharmaceutically acceptable formulations typically include one or more compounds of the invention as well as one or more pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat transplant or autoimmune disease, i.e., immunomodulatory agents and anti-inflammatory agents, as described above, can also be incorporated into the compositions of the invention. Suitable pharmaceutically active compounds that may be used can be found in Harrison's Principles of Internal Medicine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions, or sterile powders for the extemporaneous preparation of sterile injectable solutions or dispersions. For intravenous administration, suitable carriers include physiological saline, bacteriostatic water, Cremophor EI™ (BASF, Parsippany, N.J.) or phosphate buffered saline (PBS). In all cases, the pharmaceutical composition must be sterile and should be fluid to the extent that easy syringability exists. It must also be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as needed, followed by filtered sterilization. Generally, dispersions are prepared by incorporating the compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the compound of the invention can be incorporated with excipients and used in the form of tablets, troches, or capsules. Oral compositions can also include an enteric coating. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds of the invention are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds of the invention are formulated into ointments, salves, gels, or creams as generally known in the art.

The present pharmaceutical compositions can also be prepared in the form of suppositories (e.g., with conventional suppository bases such as cocoa butter and other glycerides) or retention enemas for rectal delivery.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044 and U.S. Pat. No. 5,576,018, and U.S. Pat. No. 4,883,666, the contents of all of which are incorporated herein by reference.

The compounds of the invention can also be incorporated into pharmaceutical compositions which allow for the sustained delivery of the compounds to a subject for a period of at least several weeks to a month or more. Such formulations are described in published PCT application no. WO 02/74247, incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of a compound of the invention calculated to produce the desired therapeutic effect in association with the required pharmaceutical carrier. The specification for the unit dosage forms of the invention are dictated by and directly dependent on the unique characteristics of the compound and the particular therapeutic effect to be achieved, and the limitations inherent in the art of compounding such compounds for the treatment of individuals.

This invention is further illustrated by the following examples, which should not be construed as limiting. The contents of all references, patents, patent applications cited throughout this application are incorporated herein by reference. It should be understood that the use of any of the compounds described herein are within the scope of the present invention and are intended to be encompassed by the present invention and are expressly incorporated herein for all purposes.

### EXAMPLES

### General Approach to the Synthesis of 2,5-Disubstituted Thiazoles

The synthesis of 2,5-substituted thiazoles is described in Scheme 1. Reaction of alcohol R¹—OH wherein R¹ is alkyl, aralkyl, heteroaryl, heterocyclo, or cycloalkyl with substituted 4-fluoroacetophenone 1 afforded the ether-acetophenone intermediate 2. Ether-acetophenone intermediate 2 was then converted to the corresponding bromo-acetophenone using Bu₄NBr₃, which, upon reaction with NaN₃, provided the azido-acetophenone intermediate. Hydrogenation of the azido-acetophenone intermediate afforded amine 3, followed by coupling with orthogonally protected amino acid 4 or amino diol-carboxylic acid 5 gave amide 6. As a note, compound 4 was synthesized from (S)-2-(tert-butoxycarbonylamino)-3-hydroxy-2-methylpropanoic acid in three steps in overall 52-64% yield. A synthesis of (R)-3-(tert-butoxycarbonyl)-2,2,4-trimethyloxazolidine-4-carboxylic acid is described in Clemens, J. J.; Davis, M. D.; Lynch, K. R.; Macdonald, T. L. Bioorg. Med. Chem. Lett. 2005, 15, 3568-3572. Compound 5 was synthesized from 2-amino-2-(hydroxymethyl)propane-1,3-diol in five steps in overall 30% yield, also as described in Clemens, J. J.; Davis, M. D.; Lynch, K. R.; Macdonald, T. L. Bioorg. Med. Chem. Lett. 2005, 15, 3568-3572. Under conditions using Lawesson's reagent, amide 6 was converted to thiazole 7 in good yield. Removal of the protecting groups afforded the final alcohol 8, which upon reaction with diethyl chlorophosphate and subsequent deprotection with TMSBr gave the phosphate 9. 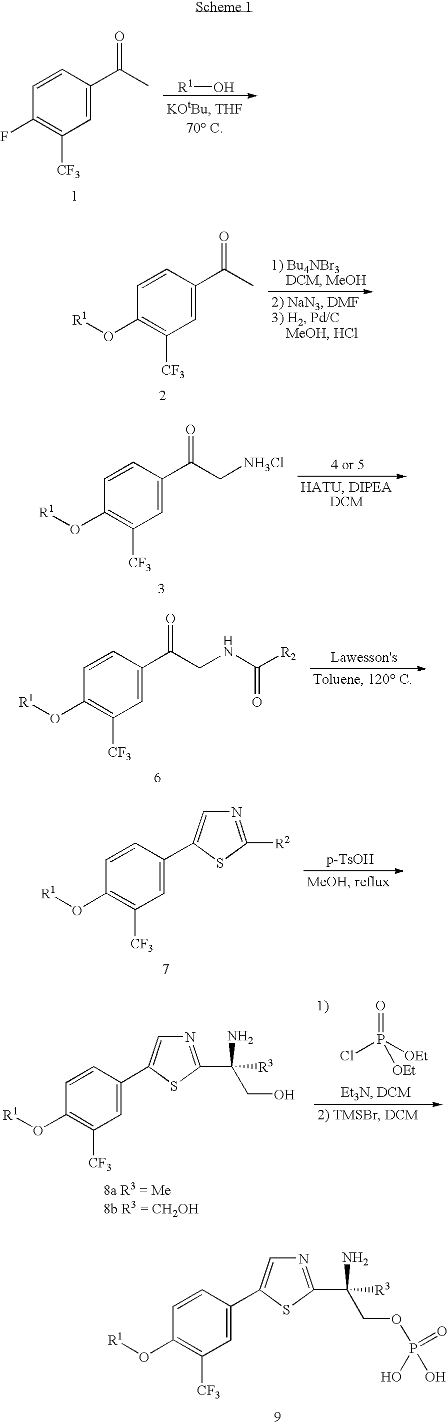 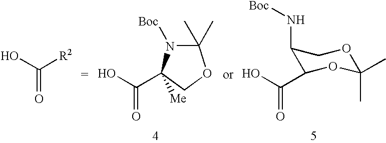
General Protocol for Synthesis of Substituted Acetophenones (Williamson Ether Synthesis) (2)

To a solution of the desired alcohol (1.0 equivalent) in dry THF under nitrogen atmosphere was added KO^{t}Bu (either 1.0 M solution in THF or solid, 1.1 equivalent). The reaction mixture was heated at 60-70° C. for 10 minutes, then substituted 4-fluoroacetophenone 1 (1.0 equivalent) was added. The reaction was then stirred for 1 to 3 hours before cooling to room temperature (RT). The solvent removed in vacuo. The product was purified by silica gel column chromatography using the Combi-Flash system (Hex:EtOAc).

### (R)-3-(tert-Butoxycarbonyl)-2,2,4-trimethyloxazolidine-4-carboxylic acid (4)

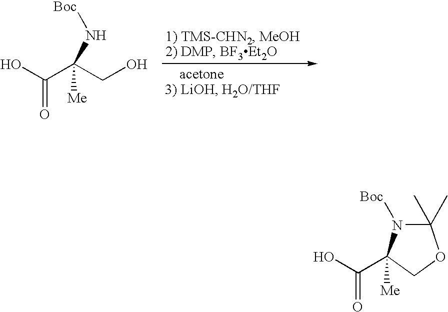

To a solution of the (S)-2-(tert-butoxycarbonylamino)-3-hydroxy-2-methylpropanoic acid (5.0 g, 1.0 equivalent) in CH₂Cl₂/MeOH (4:1, 50 mL) at 0° C. was added a solution of TMS-CHN₂ (2.0 M in diethyl ether or hexanes, 12.5 mL, 1.1 equivalents) drop-wise until the colourless solution turned a light yellow color. The reaction mixture was stirred for 20 minutes then a few drops of acetic acid were added to quench the last unreacted TMS-CHN₂ (the solution turns colorless from light yellow). The solvent was removed in vacuo. TLC (2:1, Hex/EtOAc), R_{f}=0.4.

The residue was dissolved in acetone (30 mL). To the resulting solution was then added 2,2-dimethoxypropane (DMP) (15 mL). To the mixture was added BF₃.OEt₂ (2 mL) drop-wise and the solution was stirred at room temperature (RT) for 4-18 hours. The solvent was removed in vacuo and the product was purified by silica gel column chromatography using the Combi-Flash system (Hex:EtOAc). TLC (3:1, Hex/EtOAc), R_{f}=0.6; ¹H NMR (400 MHz, CDCl₃) δ 4.06-4.12 (m, 1H), 3.73-3.83 (m, 4H), 1.55-1.64 (m, 9H), 1.48 (br s, 3H), 1.41 (br s, 6H).

The purified residue was dissolved in THF (40 mL) and to the solution was added LiOH (1.15 g, 1.20 equiv) in H₂O (20 mL). The solution was heated at reflux for 6-18 hours, then concentrated in vacuo to remove most of the THF. The solution was diluted with H₂O (150 mL) and washed with Et₂O (2×150 mL). The aqueous layer was cooled to 0° C. then acidified to a pH of approximately 3 using concentrated HCl, then extracted with EtOAc (2×200 mL). The EtOAc layers were combined, dried (MgSO₄), filtered, and the solvent was removed in vacuo to afford carboxylate 4 as a white solid in 52-64% yield (3.78 g) yield. TLC (1:1 EtOAc:Hex), R_{f}=0.2; ¹H NMR (400 MHz, CDCl₃) o (rotamers) 4.47 (br d, 0.5H, J=8.8 Hz), 4.17 (br d, 0.5H, J=8.8 Hz), 3.85 (br d, 0.5H, J=8.8 Hz), 3.78 (br d, 0.5H, J=8.8 Hz), 1.38-1.67 (m, 18H).

### 5-(tert-butoxycarbonylamino)-2,2-dimethyl-1,3-dioxane-5-carboxylic acid (5)

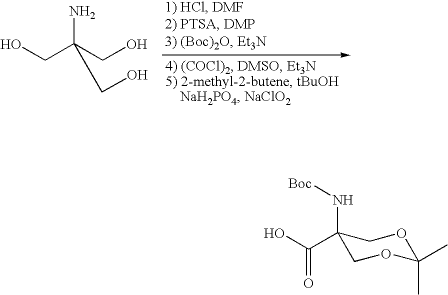

To a solution of the 2-amino-2-(hydroxymethyl)propane-1,3-diol (2.0 g, 1.0 equivalent) in DMF (20 mL) at RT was added 1M HCl (16.5 mL, 1.0 equiv) in diethyl ether. The resulting mixture was stirred for 20 minutes, then para-toluenesulfonic acid (PTSA) (157 mg, 0.05 equivalent) and 2,2-dimethoxypropane or (2.23 mL, 1.1 equivalents) were added. The reaction mixture was stirred for 24 hours, then Et₃N (3.0 equivalent, 6.90 mL) and (Boc)₂O (1.0 equiv, 3.60 g) were added and the mixture was stirred overnight. The reaction mixture was diluted with EtOAc (50 mL) and washed with H₂O (2×50 mL). The solvent removed in vacuo and the product was purified by silica gel column chromatography using the Combi-Flash system (Hex:EtOAc) as a white solid in 58% (2.49 g) yield. TLC (2:1, Hex/EtOAc), R_{f}=0.3; ¹H NMR (400 MHz, CDCl₃) δ 5.33 (br s, 1H), 4.27 (br s, 1H), 3.79-3.84 (m, 4H), 3.72 (d, 2H, J=6.4 Hz), 1.46 (s, 12H), 1.44 (s, 3H).

To a solution of oxalyl chloride (2.0 M in CH₂Cl₂, 5.74 mL, 3.0 equivalents) in dry CH₂Cl₂ (10 mL) at −78° C. was added DMSO (1.36 ml, 5.0 equivalents). The resulting mixture was stirred for 15 minutes, then a solution of the desired alcohol (from last step, 1.0 g) in dry CH₂Cl₂ (10 mL) was added drop-wise. The mixture was stirred for 2 hours, then Et₃N (5.33 mL, 10 equivalents) was added. The reaction mixture was stirred for 10 minutes then the cooling bath was removed and the mixture was allowed to warm to RT. The reaction mixture was then diluted with EtOAc (50 mL) and washed with 10% NH₄Cl (2×50 mL). The organic layer was dried over MgSO₄, filtered, and the solvent was removed in vacuo to afford aldehyde intermediate as a white solid in >99% yield (1.00 g). For more detailed Swern oxidation conditions see: a) Blaskovich, M. A.; Evindar, G.; Rose, N. G. W.; Wilkinson, S.; Luo, Y.; Lajoie, G. A. J. Org. Chem. 1998, 63, 3631-3646. and b) Rose, N. G. W.; Blaskovich, M. A.; Evindar, G.; Wilkinson, S.; Luo, Y.; Fishlock, D.; Reid, C.; Lajoie, G. A. Organic Syntheses 2002, 79, 216-227. TLC (2:1, Hex/EtOAc), R_{f}=0.7; ¹H NMR (400 MHz, CDCl₃) δ 9.63 (s, 1H), 5.55 (br s, 1H), 4.07 (d, 2H, J=12.0 Hz), 3.95 (d, 2H, J=12.0 Hz), 1.47 (s, 18H).

To a solution of the aldehyde (from last step, 1.0 g) in t-BuOH (20 mL) and 2-methyl-2-butene (10 mL) at room temperature was added a solution of NaH₂PO₄ (1.06 g, 2.0 equivalents), and NaClO₂ (1.40 g, 4.0 equivalents) in H₂O (10 mL). The reaction was stirred for 3 hours and then was diluted with H₂O (10 mL). The mixture was extracted with EtOAc (30 mL). The organic layer was dried over MgSO₄, filtered, and the solvent was removed in vacuo to afford carboxylate 5 as a white solid in 52% yield (550 mg). For more detailed procedure for oxidation of aldehyde to carboxylate see: Taylor, R. E.; Galvin, G. M.; Hilfiker, K. A.; Chen, Y. J. Org. Chem. 1998, 63, 9580-9583. TLC (1:1 EtOAc:Hex), R_{f}=0.2; ¹H NMR (400 MHz, CDCl₃) δ 5.50 (br s, 1H), 4.18 (d, 2H, J=11.8 Hz), 4.10 (d, 2H, J=11.8 Hz), 1.47 (br s, 18H).

General Approach to the Synthesis of 2,5-Disubstituted-1,3,4-Thiadiazoles

The synthesis of 2,5-substituted thiadiazoles is described in Scheme 2. Reaction of alcohol ROH with substituted 4-fluorobenzoic acid 10 afforded ether-benzoate intermediate 11. The ether-benzoate intermediate 11 was then coupled with hydrazine to afford benzohydrazide 13. Reaction of benzohydrazide 13 with orthogonally protected amino Note, this phrase used here and elsewhere in the application is new to me acid 4 under using N,N,N′-Tetramethyl-O-(7-azabenzotriazol-1-yl)uronium hexafluorophosphate (HATU) followed by cyclization with Lawesson's reagent provided thiadiazole 14 in good yield. Removal of the protecting groups afforded final alcohol 15. Alcohol 15 was then converted to corresponding phosphate as reported in scheme 1. 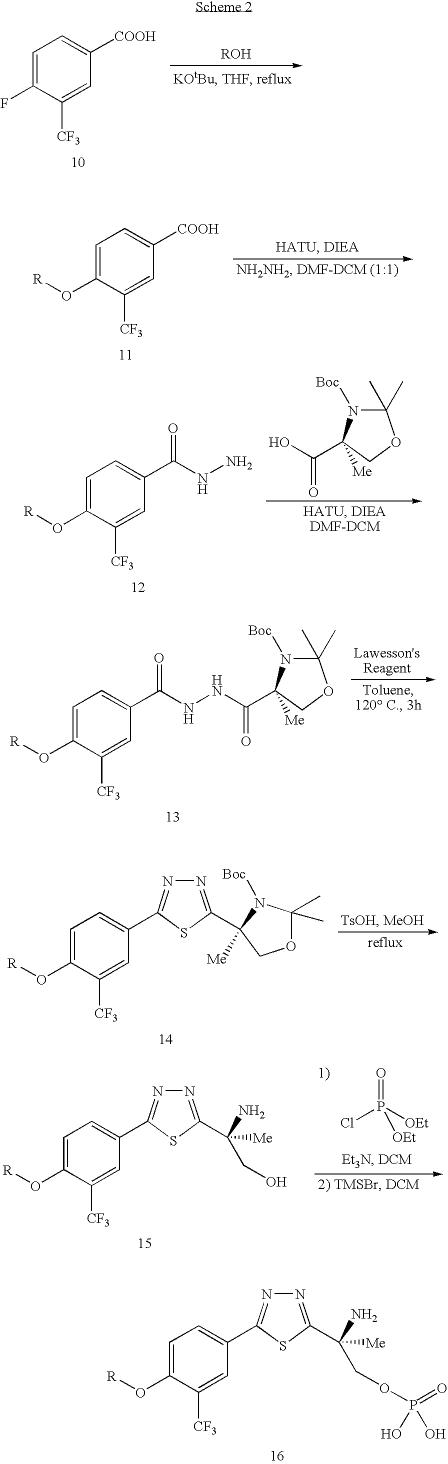
General Approach for Synthesis of Carboxylate 11

To a solution of the desired alcohol (1.0 equiv) in anhydrous THF was added potassium t-butyloxide (2.5 equiv, 1M solution in THF). The mixture was heated at 70° C. for 15 min then cooled down to room temperature. 4-Fluoro-3-trifluoromethylbenzoid acid (10) (1.0 equiv) in THF was added and the resultant was heated at 75° C. overnight. After cooling down to room temperature, the reaction was diluted with ethyl acetate and washed with water. The water layer was acidified to pH=3 with HCl (2M) and extracted with ethyl acetate. The combined organic layer was washed with brine, dried over Na₂SO₄ and concentrated in vacuo to afford the title compound which was used for next reaction without further purification.

### 4-(2-(Pentyloxy)ethoxy)-3-(trifluoromethyl)benzoic acid (11a)

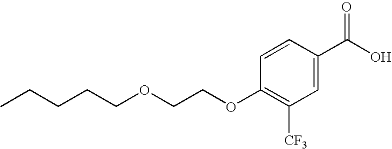

The title compound was prepared based on the general protocol for synthesis of carboxylate 11 in >95% yield. HPLC retention time on a C8(2) column (30×3.00 mm, 3μ) is 2.97 min with gradient 20-98% acetonitrile-H₂O (0.1% TFA) in 3.5 min as mobile phase.

### 4-(3-(tert-Butyldimethylsilyloxy)propoxy)-3-(trifluoromethyl)benzoic acid (11b)

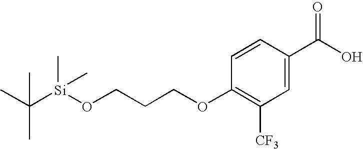

The title compound was prepared based on the general protocol for synthesis of carboxylate 11 in 68% yield. HPLC retention time on a C8(2) column (30×3.00 mm, 3μ) is 2.89 min with gradient 50-98% acetonitrile-H₂O (0.1% TFA) in 3.5 min as mobile phase.

### 4-(3-(Benzyloxy)propoxy)-3-(trifluoromethyl)benzoic acid (11c)

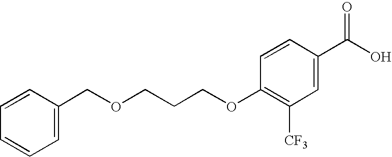

The title compound was prepared based on the general protocol for synthesis of carboxylate 11 in >95% yield. HPLC retention time on a C8(2) column (30×3.00 mm, 3μ) is 1.95 min with gradient 50-98% acetonitrile-H₂O (0.1% TFA) in 3.5 min as mobile phase.

General Approach for Synthesis of Benzohydrazide 12

The desired benzoic acid 11 (1.0 equiv) was stirred with HATU (1.0 equiv) and DIEA (5.0 equiv) in a mixture of DCM-DMF (4:1) for 10 min followed by addition of hydrazine (5.0 equiv) dropwise. The reaction mixture was continuously stirred for another hour, then was diluted with ethyl acetate and washed with water (1×) and brine (3×). The organic layer was dried over Na₂SO₄ and concentrated in vacuo to afford the desired product, which was used for next reaction without further purification.

### 4-(2-(Pentyloxy)ethoxy)-3-(trifluoromethyl)benzohydrazide (12a)

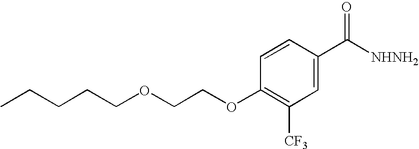

The title compound was prepared based on the general protocol for synthesis of benzohydrazide 12 in >95% yield. MS (ESI): 335.1 (MH⁺); HPLC retention time on a C8(2) column (50×3.00 mm, 3μ) is 2.82 min with gradient 20-95% acetonitrile-H₂O (0.1% TFA) in 4.0 min as mobile phase.

### 4-(3-(tert-Butyldimethylsilyloxy)propoxy)-3-(trifluoromethyl)benzohydrazide (12b)

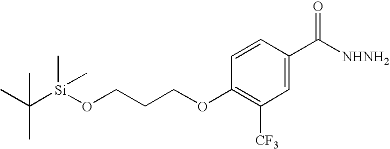

The title compound was prepared based on the general protocol for synthesis of benzohydrazide 12 in 77% yield except that column purification (silica gel, ethyl acetate-hexane (0-30%, v/v) as eluent system) was performed in order to obtain pure sample. MS (ESI): 393.09 (MH⁺); HPLC retention time on a C8(2) column (30×3.00 mm, 3μ) is 2.34 min with gradient 30-98% acetonitrile-H₂O (0.1% TFA) in 3.5 min as mobile phase.

### 4-(3-(Benzyloxy)propoxy)-3-(trifluoromethyl)benzohydrazide (12c)

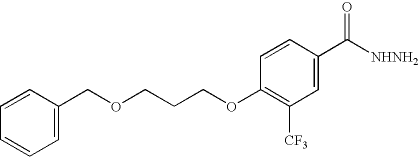

The title compound was prepared based on the general protocol for synthesis of benzohydrazide 12 in >95% yield. MS (ESI): 369.09 (MH⁺); HPLC retention time on a C8(2) column (30×3.00 mm, 3μ) is 1.81 min with gradient 30-98% acetonitrile-H₂O (0.1% TFA) in 3.5 min as mobile phase.

General Approach for Synthesis of Oxazolidine-Hydrazide 13

Carboxylic acid 4 (1.0 equiv) was stirred with HATU (1.0 equiv) and DIEA (5.0 equiv) in DCM-DMF (2:1) for 10 min followed by addition of benzohydrazide 12 (1.0 equiv) in DCM. The reaction was stirred at room temperature for 1 h and then was concentrated under vacuum. The residue was diluted with ethyl acetate and washed with water (1×), brine (2×) and dried over Na₂SO₄. The organic layer was condensed in vacuo and chromatographed on a silica gel column (ethyl acetate-hexane, 0-33%, as eluent) to afford the title compound.

### (S)-tert-Butyl 2,2,4-trimethyl-4-(2-(4-(2-(pentyloxy)ethoxy)-3-(trifluoromethyl)benzoyl)hydrazinecarbonyl)oxazolidine-3-carboxylate (13a)

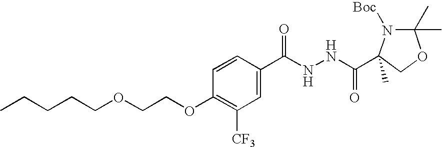

The title compound was prepared based on the general protocol for synthesis of oxazolidine-hydrazide 13 in quantitative yield. MS (ESI): 575.82 (MH⁺); ¹H NMR (400 MHz, CDCl₃) δ 9.96 (br s, 1H), 8.92 (br, 1H), 8.05 (d, 1H, J=2.4 Hz), 7.95 (dd, 1H, J=8.8 Hz, J=2.4 Hz), 7.06 (d, 1H, J=8.8 Hz), 4.55 (br s, 1H), 4.25 (t, 2H, J=4.4 Hz), 3.83 (t, 2H, J=5.2 Hz), 3.78 (br, 1H), 3.54 (t, 2H, J=6.8 Hz), 1.68 (s, 6H), 1.58 (m, 3H), 1.52 (s, 9H), 1.33-1.29 (m, 4H), 0.89 (t, 3H, J=5.6 Hz).

### (S)-tert-Butyl 4-(2-(4-(3-(tert-butyldimethylsilyloxy)propoxy)-3-(trifluoromethyl)benzoyl)hydrazinecarbonyl)-2,2,4-trimethyloxazolidine-3-carboxylate (13b)

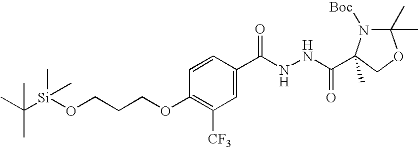

The title compound was prepared based on the general protocol for synthesis of oxazolidine-hydrazide 13 in 53% yield. MS (ESI): 633.97 (MH⁺); ¹H NMR (400 MHz, CDCl₃) δ 9.96 (br s, 1H), 8.88 (br, 1H), 8.05 (d, 1H, J=1.6 Hz), 7.95 (dd, 1H, J=8.8 Hz, J=1.6 Hz), 7.05 (d, 1H, J=8.8 Hz), 4.58 (br s, 1H), 4.20 (t, 2H, J=5.6 Hz), 3.81 (t, 2H, J=6.4 Hz), 3.79 (br s, 1H), 2.02 (m, 2H), 1.68 (s, 6H), 1.58 (m, 3H), 1.52 (s, 9H), 0.87 (s, 9H), 0.02 (s, 6H).

### (S)-tert-Butyl 4-(2-(4-(3-(benzyloxy)propoxy)-3-(trifluoromethyl)benzoyl)hydrazinecarbonyl)-2,2,4-trimethyloxazolidine-3-carboxylate (13c)

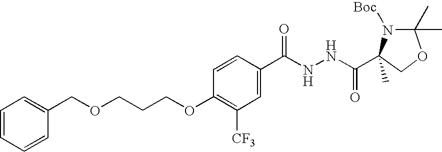

The title compound was prepared based on the general protocol for synthesis of oxazolidine-hydrazide 13 in 84% yield. MS (ESI): 609.93 (MH⁺); ¹H NMR (400 MHz, CDCl₃) δ 9.94 (br, 1H), 9.22 (br s, 1H), 8.07 (s, 1H), 7.96 (d, 1H, J=8.4 Hz), 7.29 (m, 5H), 7.00 (d, 1H, J=8.4 Hz), 4.55 (br s, 1H), 4.50 (s, 2H), 4.21 (t, 2H, J=5.6 Hz), 3.77 (br, 1H), 3.67 (t, 2H, J=6.0 2.12 (m, 2H), 1.68 (s, 6H), 1.58 (s, 3H), 1.52 (s, 9H).

General Approach for Synthesis of di-Substituted Thiadiazole 14

A solution of oxazolidine-hydrazide 13 (1.0 equiv) in toluene was treated with Lawesson's reagent (3.0 equiv) at 85° C. for 2 hours. The reaction was cooled down to room temperature and the supernatant was chromatographed on a silica gel column eluted with ethyl acetate-hexane (0-30%, v/v) to afford the title compound.

### (R)-tert-butyl 2,2,4-trimethyl-4-(5-(4-(2-(pentyloxy)ethoxy)-3-(trifluoromethyl)-phenyl)-1,3,4-thiadiazol-2-yl)oxazolidine-3-carboxylate (14a)

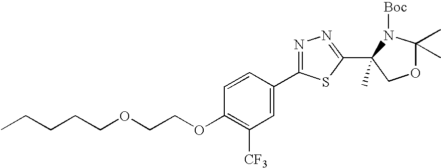

The title compound was prepared based on the general protocol for synthesis of oxazolidine-hydrazide 14 in 81% yield. MS (ESI): 574.16 (MH⁺), HPLC retention time on a C8(2) column (30×3.00 mm, 3μ) is 3.42 minutes with gradient 50-98% acetonitrile-H₂O (0.1% TFA) in 3.5 minutes as mobile phase.

### (R)-tert-Butyl 4-(5-(4-(3-hydroxypropoxy)-3-(trifluoromethyl)phenyl)-1,3,4-thiadiazol-2-yl)-2,2,4-trimethyloxazolidine-3-carboxylate (14b)

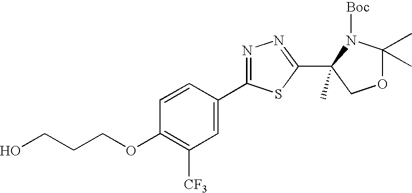

The title compound was prepared based on the general protocol for synthesis of oxazolidine-hydrazide 14 in 66% yield except that 0-70% ethyl acetate-hexane (v/v) was used as eluent system for column purification. MS (ESI): 518.13 (MH⁺), HPLC retention time on a C8(2) column (30×3.00 mm, 3μ) is 1.46 minutes with gradient 70-98% acetonitrile-H₂O (0.1% TFA) in 3.5 minutes as mobile phase.

### (R)-tert-Butyl 4-(5-(4-(3-butoxypropoxy)-3-(trifluoromethyl)phenyl)-1,3,4-thiadiazol-2-yl)-2,2,4-trimethyloxazolidine-3-carboxylate (14c)

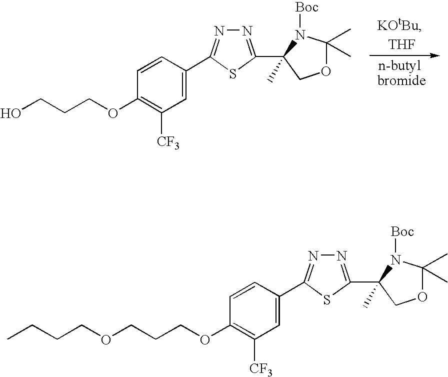

To a solution of oxazolidine-hydrazide 14b (46 mg, 0.09 mmol) in THF was added KO^{t}Bu (0.18 mL, 1M solution in THF) and the resultant was stirred at 65° C. for 10 minutes. n-Butyl bromide was added and the reaction was continuously stirred at 65° C. for overnight. The reaction mixture was cooled down to room temperature, and then was diluted with ethyl acetate and washed with water and brine. The organic layer was dried over Na₂SO₄ and condensed to provide the title compound as a crude product (52 mg) which was used for next reaction without further purification. MS (ESI): 574.25 (MH⁺), HPLC retention time on a C8(2) column (30×3.00 mm, 3μ) is 2.50 minutes with gradient 70-98% acetonitrile-H₂O (0.1% TFA) in 3.5 minutes as mobile phase.

### (R)-tert-Butyl 4-(5-(4-(3-(benzyloxy)propoxy)-3-(trifluoromethyl)phenyl)-1,3,4-thiadiazol-2-yl)-2,2,4-trimethyloxazolidine-3-carboxylate (14d)

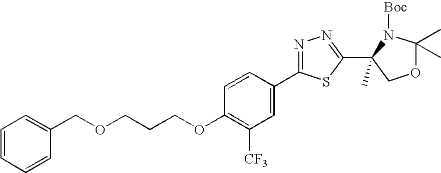

The title compound was prepared based on the general protocol for synthesis of oxazolidine-hydrazide 14 in 55% yield. MS (ESI): 608.24 (MH⁺), HPLC retention time on a C8(2) column (30×3.00 mm, 3μ) is 2.38 minutes with gradient 70-98% acetonitrile-H₂O (0.1% TFA) in 3.5 minutes as mobile phase

General Approach for Synthesis of di-Substituted Thiadiazole-Aminoalcohol 15

A solution of oxazolidine-hydrazide 14 (1.0 equiv) in methanol was treated with p-toluenesulfonic acid monohydrate (5.0 equiv) at 70° C. for 2 h. The reaction mixture was then cooled to room temperature and purified by prep HPLC on a C8(2) column (Luna, 5μ, 100×21.10 mm) with acetonitrile-H₂O (0.1% TFA) as mobile phase and gradient 30-98% in 20 minutes. The title compound was obtained as bis-TFA salt.

### (S)-2-Amino-2-(5-(4-(2-(pentyloxy)ethoxy)-3-(trifluoromethyl)phenyl)-1,3,4-thiadiazol-2-yl)propan-1-ol (15a)

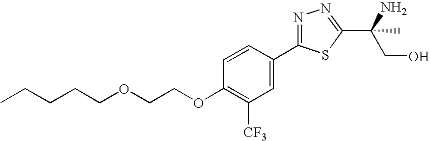

The title compound was prepared based on the general protocol for synthesis of oxazolidine-hydrazide 15 in 70% yield. MS (ESI): 434.03 (MH⁺); ¹H NMR (400 MHz, DMSO-d₆) δ 8.63 (br s, 2H), 8.23 (dd, 1H, J=8.8 Hz, J=2.4 Hz), 8.16 (d, 1H, J=2.4 Hz), 7.49 (d, 1H, J=8.8 Hz), 4.36 (t, 2H, J=4.4 Hz), 3.83 (d, 1H, J=11.2 Hz), 3.76 (d, 1H, J=11.2 Hz), 3.74 (t, 2H, J=5.2 Hz), 3.46 (t, 2H, J=6.8 Hz), 1.69 (s, 3H), 1.48 (m, 2H), 1.25 (m, 4H), 0.83 (t, 3H, J=7.2 Hz).

### (S)-2-Amino-2-(5-(4-(3-butoxypropoxy)-3-(trifluoromethyl)phenyl)-1,3,4-thiadiazol-2-yl)propan-1-ol (15b)

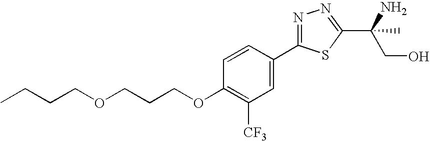

The title compound was prepared based on the general protocol for synthesis of oxazolidine-hydrazide 15 in 20% yield. MS (ESI): 434.08 (MH⁺); ¹H NMR (400 MHz, CD₃OD) δ 8.22 (d, 1H, J=2.0 Hz), 8.18 (dd, 1H, J=8.8 Hz, J=2.0 Hz), 7.37 (d, 1H, J=8.4 Hz), 4.29 (t, 2H, J=5.6 Hz), 3.97 (d, 1H, J=11.2 Hz), 3.90 (d, 1H, J=11.2 Hz), 3.63 (t, 2H, J=6.0 Hz), 3.45 (t, 2H, J=6.4 Hz), 2.08 (m, 2H), 1.82 (s, 3H), 1.54 (m, 2H), 1.36 (m, 2H), 0.89 (t, 3H, J=7.2 Hz).

### (S)-2-Amino-2-(5-(4-(3-(benzyloxy)propoxy)-3-(trifluoromethyl)phenyl)-1,3,4-thiadiazol-2-yl)propan-1-ol (15c)

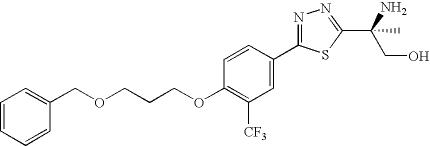

The title compound was prepared based on the general protocol for synthesis of oxazolidine-hydrazide 15 in 37% yield. MS (ESI): 468.07 (MH⁺); ¹H NMR (400 MHz, CD₃OD) δ 8.21 (d, 1H, J=2.0 Hz), 8.17 (dd, 1H, J=8.8 Hz, J=2.0 Hz), 7.36 (d, 1H, J=8.8 Hz), 7.32-7.22 (m, 5H), 4.51 (s, 2H), 4.30 (t, 2H, J=6.0 Hz), 3.97 (d, 1H, J=11.2 Hz), 3.90 (d, 1H, J=11.2 Hz), 3.70 (t, 2H, J=6.0 Hz), 2.12 (m, 2H), 1.83 (s, 3H).

General Method for Phosphate Synthesis

Synthetic strategy for synthesis of desired phosphates is illustrated in Scheme 1 above. To a solution of unprotected amino alcohol (1.0 equiv) in dry CH₂Cl₂ at room temperature was added excess diethyl chlorophosphate (10.0 equiv) and triethylamine (20.0 equivalents) and the reaction stirred for 12-18 hours. The reaction was monitored by LC-MS. The crude reaction mixture was then evaporated to dryness in vacuo. The obtained phospho-diester intermediate was reacted with excess bromotrimethylsilane (10.0-20.0 equiv) in dry CH₂Cl₂ at room temperature over a period of 6-10 hours to afford the final phosphate which was purified by reverse-phase preparative HPLC after evaporation of the solvent and excess reagent.

### (S)-2-Amino-2-(5-(4-(2-(pentyloxy)ethoxy)-3-(trifluoromethyl)phenyl)-1,3,4-thiadiazol-2-yl)propyl dihydrogen phosphate (16a)

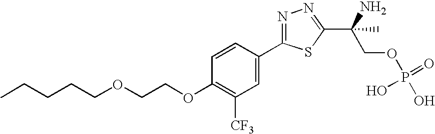 MS (ESI): 514.00 (MH⁺), HPLC retention time on a C8(2) column (30×3.00 mm, 3μ) is 1.81 min with gradient 30-98% acetonitrile-H₂O (0.1% TFA) in 3.5 min as mobile phase.
General Approach to Synthesis of Compounds of Formula III

The synthesis of compounds of formula III is described in Scheme 3. Synthesis of compounds 1a and 1b in strategy A and compound 3 in strategy B were described in schemes 1 and 2. Oxidation of the compounds 1a and 1b in strategy A followed by deprotection afforded compounds 2a and 2b. In strategy B, coupling of the free amine in compound 3 with the desired protected-amino acid gave compound 4 which upon cyclization under Lawesson's reagent conditions provided the desired azole 5. Removal of the protecting groups afforded the final carboxylate 6. 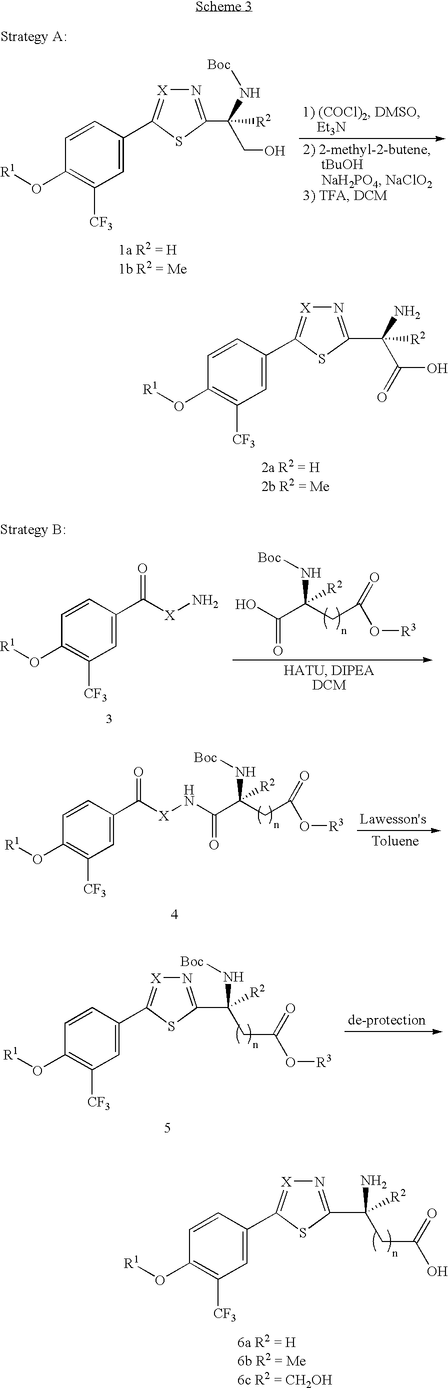
General Approach to Synthesis of Phenyl-Thiadiazoles

Synthesis of phenyl-thiazoles is described in Scheme 4. Reaction of benzyl or allyl alcohol with substituted 4-fluorobenzoic acid 1 afforded the substituted ether-benzoate 2. The substituted ether-benzoate 2 was then coupled with hydrazine to afford benzohydrazide 3. Reaction of benzohydrazide 3 with orthogonally protected amino acid 4 under HATU conditions followed by cyclization and deprotection (or vis versa) provided phenol 6 in good yield. Mitsunobu reaction of phenol 6 with desired alcohol followed by deprotection afforded the desired final compound 8. Reaction of the alcohol 8 with diethyl chlorophosphate followed by deprotection with TMSBr gave the corresponding phosphate.

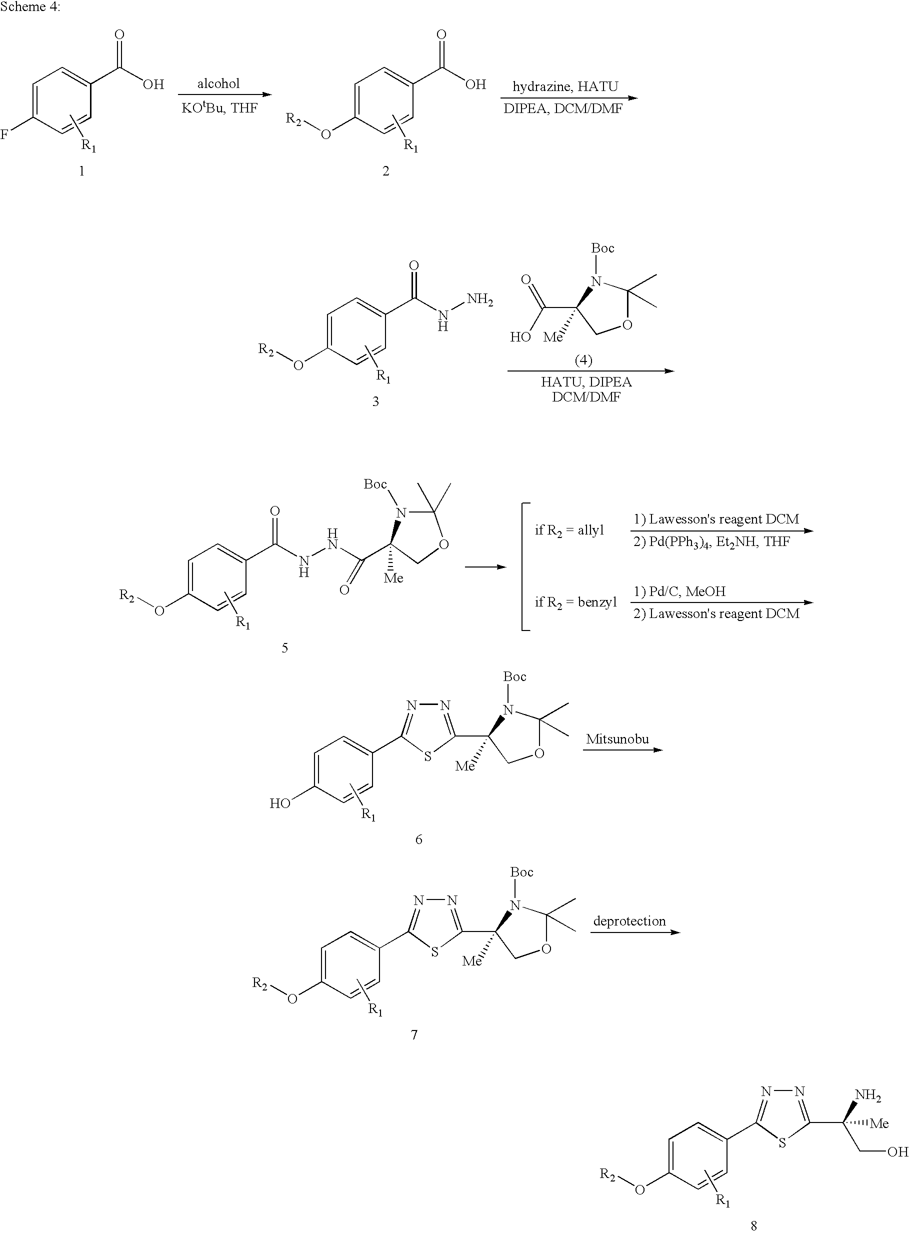

Some further aspect of R₂ modifications are described in scheme 5. Mitsunobu reaction of phenol 6 with desired diol or mono-silyl protected diol followed by deprotection of the silyl group afforded the desired alcohol 9. A second Mitsunobu reaction or alkylation provided phenyl-azole 7 which upon deprotection gave alcohol 8. 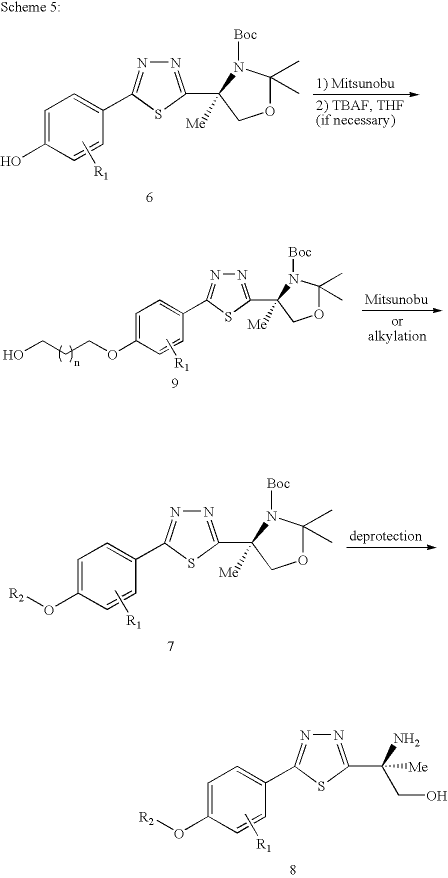
General Procedure for Synthesis of Phenoxyethoxyethanols

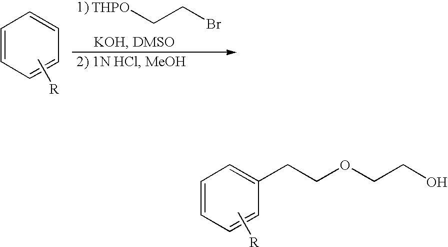

To a mixture of 2-(2-bromoethoxy)tetrahydro-2H-pyran (2 equiv) and the proper substituted 2-phenoxyethanol (1.0 equiv) in DMSO was added solid KOH (2.0 equiv). The reaction mixture was stirred at 100° C. for 3 h. After cooling to rt, the reaction mixture was quenched with water, extracted with EtOAc. The organic layer was washed with brine, dried over anhydrous Na₂SO₄, and concentrated under reduced pressure to give a residue as the substituted 2-(2-(2-phenoxyethoxy)ethoxy)tetrahydro-2H-pyran. The crude material was then treated with MeOH: 1N HCl (10:1). The reaction mixture was stirred at rt for 1 h, concentrated under reduced pressure, extracted with EtOAc. The organic layer was dried over anhydrous Na₂SO₄, concentrated under reduced pressure and purified by SiO₂ column chromatograph (n-hexane/EtOAc=4:1) to final substituted phenoxyethoxyethanol.

### 2-Phenethoxyethanol

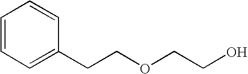

2-Phenethoxyethanol was prepared according to the general procedure as a colorless oil. HPLC retention time on a C18 column (30×4.6 mm, 3.5μ) was 1.52 min with gradient 10-95% acetonitrile-H₂O (0.1% TFA) in 3.5 min as mobile phase. MS (ESI, M+H⁺)=167.2; NMR (400 MHz, CDCl₃) δ 7.31-7.21 (m, 5H), 3.73-3.69 (m, 4H), 3.56 (t, 2H, J=4.4 Hz), 2.91 (t, 2H, J=7.2 Hz), 1.82 (br s, 1H).

### 2-(2-Chlorophenethoxy)ethanol

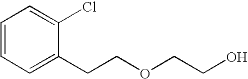

2-(2-Chlorophenethoxy)ethanol was prepared according to the general procedure as a colorless oil. HPLC retention time on a C18 column (30×4.6 mm, 3.5μ) was 1.85 min with gradient 10-95% acetonitrile-H₂O (0.1% TFA) in 3.5 min as mobile phase. MS (ESI, M+H⁺)=201.2

### 2-(3-Chlorophenethoxy)ethanol

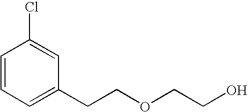

2-(3-Chlorophenethoxy)ethanol was prepared according to the general procedure as a colorless oil. HPLC retention time on a C18 column (30×4.6 mm, 3.5μ) was 1.88 min with gradient 10-95% acetonitrile-H₂O (0.1% TFA) in 3.5 min as mobile phase. MS (ESI, M+H⁺)=201.2

### 2-(4-Chlorophenethoxy)ethanol

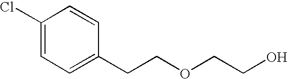

2-(4-Chlorophenethoxy)ethanol was prepared according to the general procedure as a colorless oil. HPLC retention time on a C18 column (30×4.6 mm, 3.5μ) was 1.90 min with gradient 10-95% acetonitrile-H₂O (0.1% TFA) in 3.5 min as mobile phase. MS (ESI, M+H⁺)=201.2

### 2-(2-Methoxyphenethoxy)ethanol

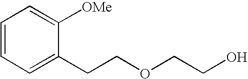

2-(2-Methoxyphenethoxy)ethanol was prepared according to the general procedure as a colorless oil. HPLC retention time on a C18 column (30×4.6 mm, 3.5μ) was 1.65 min with gradient 10-95% acetonitrile-H₂O (0.1% TFA) in 3.5 min as mobile phase. MS (ESI, M+H⁺)=197.2

### 2-(3-Methoxyphenethoxy)ethanol

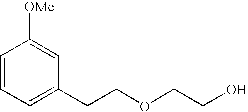

2-(3-Methoxyphenethoxy)ethanol was prepared according to the general procedure as a colorless oil. HPLC retention time on a C18 column (30×4.6 mm, 3.5μ) was 1.66 min with gradient 10-95% acetonitrile-H₂O (0.1% TFA) in 3.5 min as mobile phase. MS (ESI, M+H⁺)=197.2

### 2-(4-Methoxyphenethoxy)ethanol

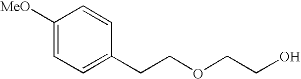

2-(4-Methoxyphenethoxy)ethanol was prepared according to the general procedure as a colorless oil. HPLC retention time on a C18 column (30×4.6 mm, 3.5μ) was 1.66 min with gradient 10-95% acetonitrile-H₂O (0.1% TFA) in 3.5 min as mobile phase. MS (ESI, M+H⁺)=197.2

### 4-Phenoxybutan-1-ol

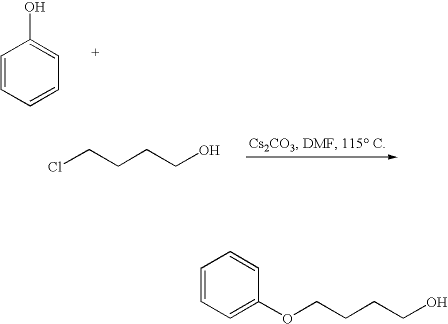

To a solution of phenol (940 mg, 10 mmol, 1 equiv) and 4-chloro-1-butanol (1.3 mL, 11 mmol, 1.1 equiv) in DMF (30 mL) was added Cs₂CO₃ (3.9 g, 12 mmol, 1.2 equiv). The reaction mixture was stirred at 115° C. for 18 h, cooled to rt, quenched with water and extracted with EtOAc. The organics was dried over anhydrous Na₂SO₄, and evaporated under reduced pressure to give a residue, which was purified by SiO₂ column chromatograph (n-hexane/EtOAc=1:9 to 1:5) to give 4-phenoxybutan-1-ol as a colorless oil (210 mg, 12%). HPLC retention time on a C18 column (30×4.6 mm, 3.5μ) was 1.78 min with gradient 10-95% acetonitrile-H₂O (0.1% TFA) in 3.5 min as mobile phase. MS (ESI, M+H⁺)=167.2; ¹H NMR (400 MHz, CDCl₃) δ 7.29-7.25 (m, 2H), 6.96-6.88 (m, 3H), 4.01 (t, 2H, J=6.2 Hz), 3.72 (t, 2H, J=6.2 Hz), 1.90-1.85 (m, 2H), 1.80-1.74 (m, 2H), 1.61 (br s, 1H).
General Protocol for Synthesis of Substituted 4-(allyloxy)benzoic acid (2)

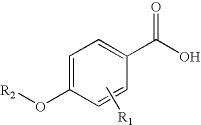

To a solution of the desired alcohol (1.05 equiv) in anhydrous THF was added potassium t-butyloxide (2.05 equiv). The mixture was heated at 65° C. for 10 minutes then added substituted 4-fluorobenzoic acid (1) (1.00 equiv) in THF. The resultant solution was heated at 65° C. 1 to 3 hours. After cooling down to room temperature, the reaction was diluted with ethyl acetate and washed with 10% KHSO₄ or 1N HCl (1×), and saturated NaCl (1×). The organic layer was dried over MgSO₄, filtered, and the solvent was removed in vacuo to afford intermediate 2.

### 4-(Allyloxy)-3-(trifluoromethyl)benzoic acid (2a)

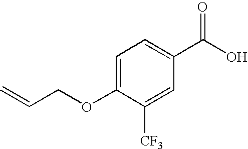

The title compound was prepared from 4-fluoro-3-(trifluoromethyl)benzoic acid (1a) in >99% (5.65 g) yield. HPLC retention time on a C8(2) column (30×3.00 mm, 3μ) was 2.53 min with gradient 20-98% acetonitrile-H₂O (0.1% TFA) in 4.0 min as mobile phase. ¹H NMR (400 MHz, DMSO-d₆) δ 13.10 (br s, 1H), 8.15 (dd, 1H, J=8.8 Hz, J=2.4 Hz), 8.08 (d, 1H, J=2.4 Hz), 7.35 (d, 1H, J=8.8 Hz), 5.95-6.80 (m, 1H), 5.38-5.45 (m, 1H), 5.26-5.32 (m, 1H), 4.77-4.82 (m, 2H).

### 4-(Benzyloxy)-3-(trifluoromethyl)benzoic acid (2b)

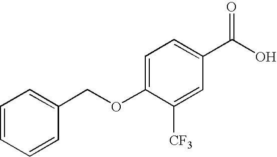

The title compound was prepared from 4-fluoro-3-(trifluoromethyl)benzoic acid (1a) in >99% (7.22 g) yield
General Protocol for Synthesis of Substituted benzohydrazide (3)

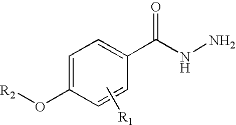

Benzoic acid 2 (1.0 equiv) was stirred with HATU (1.1 equiv) and DIEA (3.0 equiv) in DCM-DMF (2:1) for 20 minutes. The solution was then added to a solution of hydrazine mono-hydrate (3.0-5.0) in DCM-DMF (2:1). The reaction mixture was stirred at rt for 1 hour, then diluted with ethyl acetate and washed with 10% NH₄Cl (2×) and saturated NaCl (1×). The organic layer was dried over MgSO₄, filtered, and the solvent was removed in vacuo to afford benzohydrazide 3.

### 4-(Allyloxy)-3-(trifluoromethyl)benzohydrazide (3a)

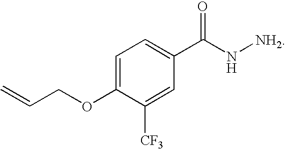

The title compound was prepared from 4-(allyloxy)-3-(trifluoromethyl)benzoic acid (1a) in >99% (6.00 g) yield. HPLC retention time on a C8(2) column (30×3.00 mm, 3μ) was 1.79 min with gradient 20-98% acetonitrile-H₂O (0.1% TFA) in 4.0 min as mobile phase. MS (ESI, M+H⁺)=261.1

### 4-(Benzyloxy)-3-(trifluoromethyl)benzohydrazide (3b)

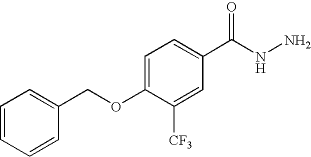

The title compound was prepared from 4-(Benzyloxy)-3-(trifluoromethyl)benzoic acid 2b in >99% (14.7 g) yield. MS (ESI, M+H⁺)=311.1.
General Protocol for Synthesis of acyl-benzohydrazide (5)

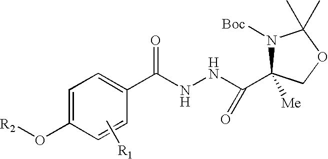

(R)-3-(tert-Butoxycarbonyl)-2,2,4-trimethyloxazolidine-4-carboxylic acid 4 (1.0 equiv) was stirred with HATU (1.1 equiv) and DIEA (3.0 equiv) in DCM-DMF (2:1) for 10 min followed by addition of substituted benzohydrazide 3 (1.0 equiv). The reaction mixture was stirred at rt for 1 hour, then diluted with ethyl acetate and washed with 10% NH₄Cl (2×) and saturated NaCl (1×). The organic layer was dried over MgSO₄, filtered, and the solvent was removed in vacuo to afford acyl-benzohydrazide 5.

### (R)-tert-Butyl 4-(2-(4-(allyloxy)-3-(trifluoromethyl)benzoyl)hydrazinecarbonyl)-2,2,4-trimethyloxazolidine-3-carboxylate (5a)

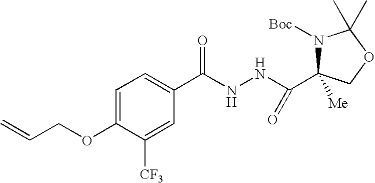

The title compound was prepared from 4-(allyloxy)-3-(trifluoromethyl)benzohydrazide 3a in >99% (11.51 g) yield. HPLC retention time on a C8(2) column (30×3.00 mm, 3μ) was 2.92 min with gradient 20-98% acetonitrile-H₂O (0.1% TFA) in 4.0 min as mobile phase. MS (ESI, M+Na⁺)=524.1

### (R)-tert-Butyl 4-(2-(4-(benzyloxy)-3-(trifluoromethyl)benzoyl)hydrazinecarbonyl)-2,2,4-trimethyloxazolidine-3-carboxylate (5b)

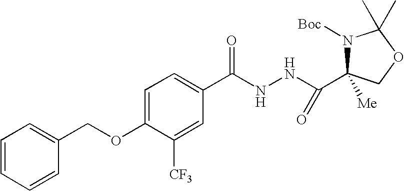

The title compound was prepared from 4-(benzyloxy)-3-(trifluoromethyl)benzohydrazide 3b in 83% (13.1 g) yield. MS (ESI, M+Na⁺)=574.1; TLC (2:1, Hex/EtOAc), R_{f}=0.34.
General Protocol for Synthesis of phenyl-thiadiazole from allyl Protected Precursor (6)

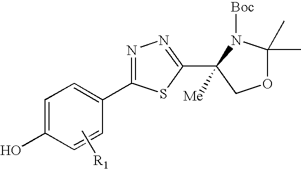

A solution of allyl protected acyl-benzohydrazide 5a (1.0 equiv) in DCM was treated with Lawesson's reagent (1.0 equiv) at 50° C. overnight. The reaction was cooled down to room temperature and the supernatant was chromatographed on a silica gel column eluted with ethyl acetate in hexanes (0-40%, v/v) to afford phenyl-thiadiazole.

A solution of phenyl-thiadiazole (1.0 equiv) and Et₂NH (1.5 equiv) in THF was treated with Pd(PPh₃)₄ (0.02 to 0.05 equiv) at rt for 1-3 hours. The solvent removed in vacuo and the product was purified by silica gel column chromatography using the Combi-Flash system (Hex:EtOAc).

### (R)-tert-Butyl 4-(5-(4-(allyloxy)-3-(trifluoromethyl)phenyl)-1,3,4-thiadiazol-2-yl)-2,2,4-trimethyloxazolidine-3-carboxylate

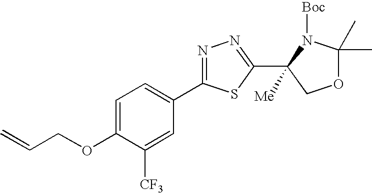

The title compound was prepared from acyl-benzohydrazide 5a in 88% (8.35 g) yield. HPLC retention time on a C8(2) column (30×3.00 mm, 3 μl) was 2.84 min with gradient 50-98% acetonitrile-H₂O (0.1% TFA) in 4.0 min as mobile phase. MS (ESI, M+H⁺)=500.0; ¹H NMR (400 MHz, DMSO-d₆) δ 8.13-8.24 (m, 2H), 7.42 (d, 1H, J=8.4 Hz), 5.98-6.10 (m, 1H), 5.40-5.50 (m, 1H), 5.25-5.34 (m, 1H), 4.80-4.84 (m, 2H), 4.10-4.40 (m, 2H), 1.88 (s, 3H), 1.66 (s, 3H), 1.56 (s, 3H), 1.41 (s, 3H), 1.18 (s, 6H).

### (R)-tert-Butyl 4-(5-(4-hydroxy-3-(trifluoromethyl)phenyl)-1,3,4-thiadiazol-2-yl)-2,2,4-trimethyloxazolidine-3-carboxylate (6a)

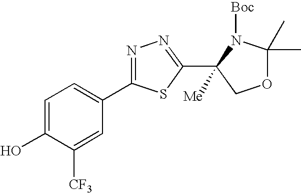

The title compound was prepared from allyl protected phenyl-thiadiazole 6a in 64% (4.86 g) yield. HPLC retention time on a C8(2) column (30×3.00 mm, 3μ) was 2.06 min with gradient 50-98% acetonitrile-H₂O (0.1% TFA) in 4.0 min as mobile phase. MS (ESI, M+H⁺)=460.0; ¹H NMR (400 MHz, DMSO-d₆) δ 11.40 (s, 1H), 8.00-8.10 (m, 2H), 7.18 (d, 1H, J=8.8 Hz), 4.07-4.21 (m, 2H), 1.88 (s, 3H), 1.67 (s, 3H), 1.57 (s, 3H), 1.42 (s, 3H), 1.19 (s, 6H).

### (R)-tert-Butyl 4-(2-(4-hydroxy-3-(trifluoromethyl)benzoyl)hydrazinecarbonyl)-2,2,4-trimethyloxazolidine-3-carboxylate (5c)

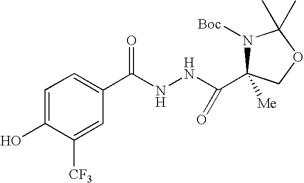

A solution of benzyl protected acyl-benzohydrazide 5b (1.0 equiv) in MeOH was subjected to hydrogenation in the presence of Pd/C (10% w) for 1 h. The reaction mixture was filtered through celite and concentrated to give compound 5c (6.86 g, 99% yield). MS (ESI, M+Na⁺): 484.0; TLC (2:1, Hex/EtOAc), R_{f}=0.20; ¹H NMR (400 MHz, CD₃OD) δ 8.11 (s, 1H,), 7.95 (d, 1H, J=8.8 Hz), 7.01 (d, 1H, J=8.8 Hz), 4.28 (br s, 1H), 3.91 (br s, 1H), 1.69 (s, 3H,), 1.64 (s, 3H), 1.58 (s, 3H), 1.49 (s, 9H).

### (R)-tert-Butyl 4-(5-(4-hydroxy-3-(trifluoromethyl)phenyl)-1,3,4-thiadiazol-2-yl)-2,2,4-trimethyloxazolidine-3-carboxylate (6a)

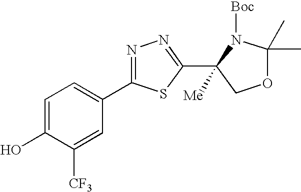 The phenyl-thiazole 6a was prepared through two different protocols from 5c:

Protocol A:

A solution of acyl-benzohydrazide 5c (1 equiv) in DCM was treated with Lawesson's reagent (3.0 equiv) at 50° C. overnight. The reaction was cooled down to room temperature and the supernatant was chromatographed on a silica gel column eluted with ethyl acetate in hexanes (0-40%, v/v) to afford phenyl-thiadiazole 6 in 37% (670 mg) yield.

Protocol B:

A solution of acyl-benzohydrazide 5c (1 equiv) in DCM was added acetyl anhydride (1.1 equiv) and pyridine (1.1 equiv). The mixture was stirred at rt for 4 h. The solvent was removed under vacuum and the residue was dissolved in ethyl acetate, washed with brine (3×), dried over MgSO₄ and concentrated to afford crude acylated 5c in quantitative (5.46 g) yield. TLC (4:1, Hex/EtOAc), R_{f}=0.40; MS (ESI, M+H⁺)=504.1.

To a solution of acylated intermediate 5c (1.0 equiv) in toluene was added Lawesson's reagent (1.1 equiv). The mixture was heated at 85° C. for 3 h. The reaction was cooled down to room temperature and the supernatant was chromatographed on a silica gel column eluted with ethyl acetate in hexanes (15%-30%, v/v) to afford acylated 6a in 82% (5.1 g) yield. MS (ESI, M+H⁺)=502.0.

Acylated 6a was dissolved in a mixture of methanol and saturated NaHCO₃ (2:1, v/v) and stirred at rt overnight. The methanol was removed and the aqueous phase was extracted with ethyl acetate (3×). The combined organic phase was washed with brine, dried over MgSO₄ and concentrated to afford 6a in 76% (3.1 g) yield.

General Protocol for Mitsunobu Reaction (Compounds 7 or 9)

To a suspension of triphenyl phosphine, polymer bound [3 mmol/g loading] (1.2-6.0 equiv) in DCM or PPh₃ (1.0 equiv) in DCM or THF, was added a phenol 6 (1.0 equiv) and the desired alcohol (1.0 equiv). The reaction was then cooled to 0° C. in an ice bath and added diisopropyl azodicarboxylate (DLAD) (1.0 equiv). The reaction was then allowed to warm to rt and stirred for 4-12 hours. The reaction mixture was filtered and the filtrate was concentrated under reduced pressure to give a crude product, which was taken on to the next step without any further purification.

General Protocol Silyl Group Deprotection (9)

A crude solution of silyl protected intermediate 9 in THF was treated with tetra-n-butylammonium fluoride (TBAF) (1.1 equiv) at 0° C. and allowed to warm to rt for 1 hour. The reaction was concentrated in vacuo and the residue was purified by silica gel column chromatography using the Combi-Flash system (Hex:EtOAc).

General Protocol for Alkylation of Compound 9 to 7

To solution of alcohol 9 in THF, added potassium t-butyloxide (1M in THF, 10 equiv) and alkyl bromide (in some cases iodide used instead of bromide, 10 equiv). The mixture was stirred at 50° C. for 24-72 h. The reaction was cooled down to rt, then diluted with ethyl acetate and washed with water (2×) and saturated NaCl (1×). The organic layer was dried over MgSO₄, filtered, and the solvent was removed in vacuo to afford compound 7 which was used directly for next step.

### (R)-tert-Butyl 4-(5-(4-(4-hydroxybutoxy)-3-(trifluoromethyl)phenyl)-1,3,4-thiadiazol-2-yl)-2,2,4-trimethyloxazolidine-3-carboxylate (9a)

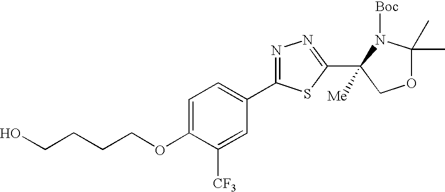

The title product was obtained from 4-(tert-butyldimethylsilyloxy)butan-1-ol according to general procedures in 76% yield. HPLC retention time on a Synergi MAX-RP 100A (20×2 mm, 2μ) was 1.41 min with gradient 40-99% acetonitrile-H₂O (0.1% TFA) in 2.0 min as mobile phase. MS (ESI, M+H⁺)=532.0; ¹H NMR (400 MHz, CD₃OD) δ 8.18 (s, 1H), 8.11 (d, 1H, J=7.6 Hz), 7.33 (d, 1H, J=8.4 Hz), 4.23 (t, 2H, J=7.4 Hz), 4.18 (br s, 1H), 3.64 (t, 2H, J=6.8 Hz), 3.56 (t, 1H, J=6.4 Hz), 3.45 (br s, 1H), 1.98 (s, 3H), 1.94-1.88 (br s, 2H), 1.78-1.71 (m, 2H), 1.64 (s, 6H), 1.25 (d, 9H).

### (R)-tert-Butyl 4-(5-(4-(5-hydroxypentyloxy)-3-(trifluoromethyl)phenyl)-1,3,4-thiadiazol-2-yl)-2,2,4-trimethyloxazolidine-3-carboxylate (9b)

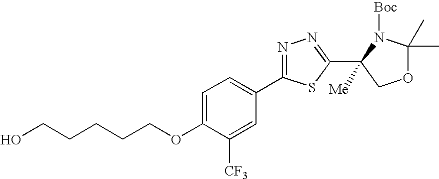

The title compound was obtained from pentane-1,5-diol in 54% (320 mg) yield. MS (ESI, MH⁺)=546.1; ¹H NMR (400 MHz, CDCl₃) δ 8.12-8.07 (m, 2H,), 7.07 (d, 1H, J=8.0 Hz), 4.19-4.07 (m, 4H), 3.69 (t, 2H, J=6.4 Hz), 1.99-1.13 (m, 24H).

### (R)-tert-Butyl 4-(5-(4-(6-hydroxyhexyloxy)-3-(trifluoromethyl)phenyl)-1,3,4-thiadiazol-2-yl)-2,2,4-trimethyloxazolidine-3-carboxylate (9c)

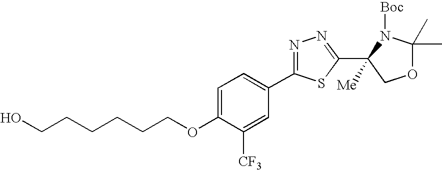

The title compound was obtained from hexane-1,6-diol in 66% (250 mg) yield. MS (ESI, MH⁺): 560.0; ¹H NMR (400 MHz, CDCl₃) δ 8.13-8.08 (m, 2H,), 7.07 (d, 1H, J=9.2 Hz), 4.19-4.07 (m, 4H), 3.67 (t, 2H, J=6.4 Hz), 1.99-1.13 (m, 26H).
General Protocol for One Pot Deprotection of Both Boc and Oxazolidine (8)

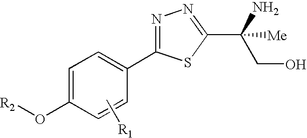

To a solution of compound 8 in DCM added TFA (10-50% v/v) and 1% anisole or triisopropyl silane (TIPS) as scavenger. The reaction mixture was allowed to stir at rt for 0.5-2 hours, dried under vacuum and was subjected directly to prep HPLC purification. The product was purified by prep HPLC on a C8(2) column ((Luna, 5μ, 100×21.10 mm) with acetonitrile-H₂O (0.1% TFA) as mobile phase and gradient 30-98% in 20 min.

### (S)-2-Amino-2-(5-(4-(2-phenethoxyethoxy)-3-(trifluoromethyl)phenyl)-1,3,4-thiadiazol-2-yl)propan-1-ol (8a)

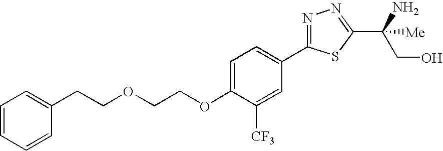

The title product was obtained according to general procedure from compound 6a (Scheme 4) in 49% (23 mg) yield. HPLC retention time on a C18 column (30×4.6 mm, 3.5μ) was 1.85 min with gradient 10-95% acetonitrile-H₂O (0.1% TFA) in 3.5 min as mobile phase. MS (ESI, M+H⁺)=468.3; ¹H NMR (400 MHz, DMSO-d6) δ 8.23-8.20 (m, 1H), 8.17-8.16 (m, 1H), 7.47 (d, 1H, J=9.2 Hz), 7.23-7.21 (m, 3H), 7.18-7.15 (m, 1H), 6.08 (t, 1H, J=5.2 Hz), 4.36 (t, 2H, J=4.6 Hz), 3.84-3.75 (m, 4H), 3.70 (t, 2H, J=6.8 Hz), 3.33 (br s, 2H), 2.81 (t, 2H, J=6.8 Hz), 1.70 (s, 3H).

### (S)-2-Amino-2-(5-(4-(2-(2-chlorophenethoxy)ethoxy)-3-(trifluoromethyl)phenyl)-1,3,4-thiadiazol-2-yl)propan-1-ol (8b)

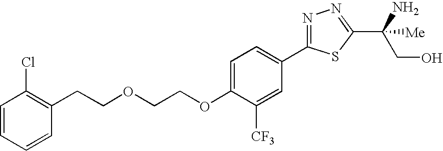

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a (Scheme 4) in 25% (125 mg). HPLC retention time on a C8(2) column (30×50 mm, 3 μL) is 1.85 min with gradient 30-98% acetonitrile-H₂O (0.1% TFA) in 3.5 min as mobile phase. MS (ESI, M+H⁺)=502.1; ¹H NMR (400 MHz, CD₃OD) δ 8.21 (d, 1H, J=2.4 HZ), 8.14 (dd, 1H, J=9.0 Hz, J=2.0 Hz), 7.37 (d, 1H, J=8.4 Hz), 7.29-7.33 (m, 2H), 7.15-7.18 (m, 2H), 4.32-4.35 (m, 2H), 3.86-4.00 (m, 4H), 3.79 (t, 2H, J=6.8 Hz), 3.02 (t, 2H, J=6.8 Hz), 1.83 (s, 3H).

### (S)-2-Amino-2-(5-(4-(2-(4-chlorophenethoxy)ethoxy)-3-(trifluoromethyl)phenyl)-1,3,4-thiadiazol-2-yl)propan-1-ol (8c)

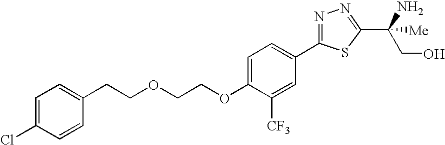

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a (Scheme 4) in 22% (108 mg). HPLC retention time on a C8(2) column (30×50 mm, 3 μL) is 1.86 min with gradient 30-98% acetonitrile-H₂O (0.1% TFA) in 3.5 min as mobile phase. MS (ESI, M+H⁺)=502.1; ¹H NMR (400 MHz, CD₃OD) δ 8.21 (d, 1H, J=2.4 Hz), 8.13 (dd, 1H, J=8.0 Hz, J=2.0 Hz), 7.34 (d, 1H, J=8.8 Hz), 7.18-7.20 (m, 4H), 4.30-4.34 (m, 2H), 3.75-3.99 (m, 4H), 2.84 (t, 2H, J=6.4 Hz), 1.83 (s, 3H).

### (S)-2-Amino-2-(5-(4-(2-phenoxyethoxy)-3-(trifluoromethyl)phenyl)-1,3,4-thiadiazol-2-yl)propan-1-ol (8d)

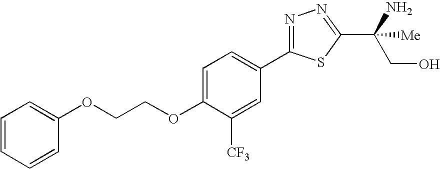

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a (Scheme 4) in 39% (171 mg). HPLC retention time on a Synergi-Max RP column (2×20 mm, 2 μL) is 1.42 min with gradient 20-95% acetonitrile-H₂O (0.1% TFA) in 2 min as mobile phase. MS (ESI, M+H⁺)=440.0; ¹H NMR (400 MHz, DMSO-d₆) δ 8.89 (br s, 2H), 8.26 (dd, 1H, J=8.0 Hz, J=2.0 Hz), 8.19 (s, 1H), 7.56 (d, 1H, J=8.8 Hz), 7.28-7.32 (m, 2H), 6.94-6.98 (m, 3H), 6.12 (s, 1H), 4.59-4.61 (m, 2H), 4.35-4.37 (m, 2H), 4.38-4.85 (m, 2H), 1.71 (s, 3H).

### (S)-2-Amino-2-(5-(4-(3-phenoxypropoxy)-3-(trifluoromethyl)phenyl)-1,3,4-thiadiazol-2-yl)propan-1-ol (8e)

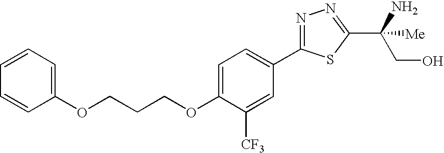

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a in 53% (238 mg). HPLC retention time on a Synergi-Max RP column (2×20 mm, 2 μL) is 1.49 min with gradient 20-95% acetonitrile-H₂O (0.1% TFA) in 2 min as mobile phase. MS (ESI, M+H⁺)=454.1; ¹H NMR (400 MHz, DMSO-d₆) δ 8.90 (br s, 2H), 8.24 (dd, 1H, J=8.8 Hz, J=1.6 Hz), 8.18 (s, 1H), 7.52 (d, 1H, J=8.8 Hz), 7.26-7.30 (m, 2H), 6.91-6.95 (m, 3H), 6.12 (br s, 1H), 4.40 (t, 2H, J=6 Hz), 4.15 (t, 2H, J=6.4 Hz), 3.74-3.81 (m, 2H), 2.21-2.26 (m, 2H), 1.71 (s, 3H).

### (S)-2-Amino-2-(5-(4-(2-(2-methoxyphenethoxy)ethoxy)-3-(trifluoromethyl)phenyl)-1,3,4-thiadiazol-2-yl)propan-1-ol (8f)

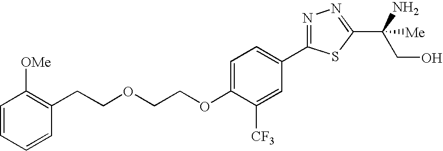

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a in 21% (106 mg). HPLC retention time on a C8(2) column (30×50 mm, 3 μL) is 1.85 min with gradient 30-98% acetonitrile-H₂O (0.1% TFA) in 3.5 min as mobile phase. MS (ESI, M+H⁺)=498.1; ¹H NMR (400 MHz, DMSO-d₆) δ 8.71 (br s, 2H), 8.21 (dd, 1H, J=9.0 Hz, J=2.0 Hz), 8.17 (d, 1H, J=2 Hz), 7.48 (d, 1H, J=9.2 Hz), 7.14-7.20 (m, 2H), 6.93 (d, 1H, J=7.6 Hz), 6.81-6.84 (m, 1H), 6.07 (t, 1H, J=4.8 Hz), 4.36 (t, 2H, J=4.4 Hz), 3.70-3.84 (m, 7H), 3.62-3.65 (m, 2H), 2.79 (t, 2H, J=7.2 Hz), 1.70 (s, 3H).

### (S)-2-Amino-2-(5-(4-(2-(3-methoxyphen ethoxy)ethoxy)-3-(trifluoromethyl)phenyl)-1,3,4-thiadiazol-2-yl)propan-1-ol (8g)

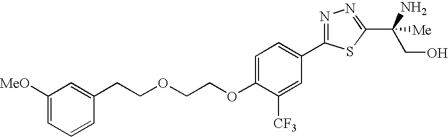

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a in 33% (165 mg). HPLC retention time on a C8(2) column (30×50 mm, 3 μL) is 1.81 min with gradient 30-98% acetonitrile-H₂O (0.1% TFA) in 3.5 min as mobile phase. MS (ESI, M+H⁺)=498.1; ¹H NMR (400 MHz, DMSO-d₆) δ 8.83 (br s, 2H), 8.21 (d, 1H, J=9.2 Hz), 8.17 (s, 1H), 7.47 (d, 1H, J=9.2), 7.16 (t, 1H, J=8 Hz), 6.73-6.80 (m, 3H), 6.09 (br s, 1H), 4.36 (t, 2H, J=4 Hz), 3.78-3.83 (m, 4H), 3.68-3.72 (m, 6H), 2.78 (t, 2H, J=7.2 Hz), 1.71 (s, 3H).

### (S)-2-Amino-2-(5-(4-(2-(4-methoxyphenethoxy)ethoxy)-3-(trifluoromethyl)phenyl)-1,3,4-thiadiazol-2-yl)propan-1-ol (8h)

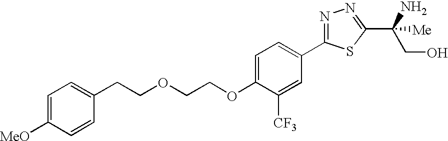

The title compound was prepared from 4-(5-(4-hydroxy-3-(trifluoromethyl)phenyl)-1,3,4-thiadiazol-2-yl)-2,2,4-trimethyoxyazolidine-3-carboxylate 6a in 25% (123 mg). HPLC retention time on a C8(2) column (30×50 mm, 3 μL) is 1.79 min with gradient 30-98% acetonitrile-H₂O (0.1% TFA) in 3.5 min as mobile phase. MS (ESI, M+H⁺)=598.1; ¹H NMR (400 MHz, DMSO-d₆) δ 8.76 (br s, 2H), 8.19 (d, 1H, J=8.4 Hz), 8.16 (s, 1H), 7.45 (d, 1H, J=8.8 Hz), 7.11 (d, 2H, J=8 Hz), 6.78 (d, 2H, J=7.6 Hz), 6.07 (t, 1H, J=4.8 Hz), 4.34 (t, 2H, J=3.6 Hz), 3.74-3.81 (m, 3H), 3.62-3.69 (m, 6H), 2.72 (t, 2H, J=7.2 Hz), 1.69 (s, 3H).

### (S)-2-Amino-2-(5-(4-(2-(3-phenylpropoxy)ethoxy)-3-(trifluoromethyl)phenyl)-1,3,4-thiadiazol-2-yl)propan-1-ol (8i)

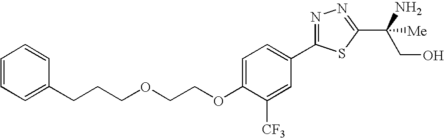

The title compound was prepared from protected phenyl-thiadiazole 6a in 26% (36 mg) yield. HPLC retention time on a C8(2) column (30×50 mm, 3 μL) is 1.90 min with gradient 30-98% acetonitrile-H₂O (0.1% TFA) in 3.5 min as mobile phase. MS (ESI, M+H⁺)=482.0; ¹H NMR (400 MHz, CD₃OD) δ 8.23 (d, 1H, J=2.0 Hz), 8.19 (dd, 1H, J=8.4 Hz, J=2.0 Hz), 7.42 (d, 1H, J=8.4 Hz), 7.21 (m, 2H), 7.12 (m, 3H), 4.36 (m, 2H), 3.98 (d, 1H, J=11.6 Hz); 3.90 (d, 1H, J=11.6 Hz); 3.84 (m, 2H), 3.53 (t, 2H, J=6.0 Hz), 2.65 (t, 2H, J=8.0 Hz), 1.86 (m, 2H), 1.83 (s, 3H).

### (S)-2-Amino-2-(5-(3-(trifluoromethyl)-4-(2-(4-(trifluoromethyl)phenethoxy)ethoxy)phenyl)-1,3,4-thiadiazol-2-yl)propan-1-ol (8j)

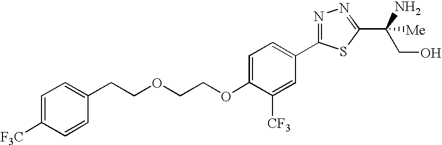

The title compound was prepared from protected phenyl-thiadiazole 6a in 72% (91 mg) yield. HPLC retention time on a C8(2) column (30×50 mm, 3 μL) is 1.90 min with gradient 30-98% acetonitrile-H₂O (0.1% TFA) in 3.5 min as mobile phase. MS (ESI, M+H⁺)=482.0; ¹H NMR (400 MHz, CD₃OD) δ 8.21 (d, 1H, J=2.0 Hz), 8.14 (dd, 1H, J=8.4 Hz, J=2.0 Hz), 7.49 (d, 2H, J=8.0 Hz), 7.40 (d, 2H, J=8.0 Hz), 7.35 (d, 1H, J=8.8 Hz), 4.31 (m, 2H), 3.98 (d, 1H, J=11.2 Hz); 3.91 (d, 1H, J=11.2 Hz); 3.86 (m, 2H), 3.82 (t, 2H, J=6.4 Hz), 2.95 (t, 2H, J=6.4 Hz), 1.83 (s, 3H).

### (S)-2-Amino-2-(5-(4-(4-phenoxybutoxy)-3-(trifluoromethyl)phenyl)-1,3,4-thiadiazol-2-yl)propan-1-ol (8k)

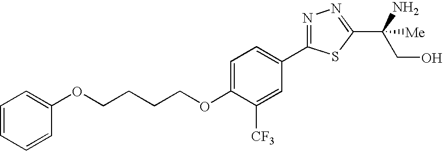

The title product was obtained according to general procedure (Scheme 4). HPLC retention time on a C18 column (30×4.6 mm, 3.5μ) was 2.12 min with gradient 10-95% acetonitrile-H₂O (0.1% TFA) in 3.5 min as mobile phase. MS (ESI, M+H⁺)=468.4; NMR (400 MHz, DMSO-d6) δ 8.87 (s, 2H), 8.25-8.17 (m, 2H), 7.48 (d, 1H, J=9.2 Hz), 7.29-7.25 (m, 2H), 6.93-6.89 (m, 3H), 6.10 (s, 1H), 4.31 (t, 2H, J=5.6 Hz), 4.04 (t, 2H, J=5.8 Hz), 3.85-3.76 (m, 2H), 1.93-1.89 (m, 4H), 1.71 (s, 3H).

### (S)-2-Amino-2-(5-(4-(4-(4-fluorophenoxy)butoxy)-3-(trifluoromethyl)phenyl)-1,3,4-thiadiazol-2-yl)propan-1-ol (8l)

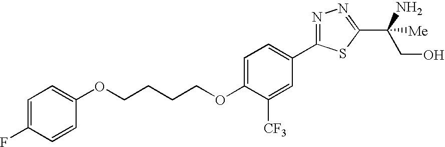

The title product was obtained according to general procedure (Scheme 5) in 21% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=486.1; ¹H NMR (400 MHz, CD₃OD) δ 8.22 (d, 1H, J=2.0 Hz), 8.17 (d, 1H, J=2.0 Hz), 7.37 (s, 1H, J=8.4 Hz), 7-6.94 (m, 2H), 6.9-6.86 (m, 2H), 4.3-4.27 (t, 2H, J=6.8 Hz), 4.05-4.02 (t, 2H, J=6.4 Hz), 3.99-3.89 (m, 2H), 2.07-1.95 (m, 4H), 1.82 (s, 3H).

### (S)-2-Amino-2-(5-(4-(4-(2-fluorophenoxy)butoxy)-3-(trifluoromethyl)phenyl)-1,3,4-thiadiazol-2-yl)propan-1-ol (8m)

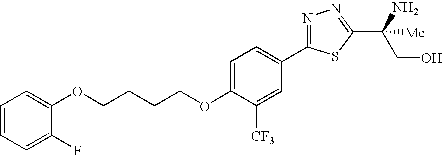

The title product was obtained according to general procedure (Scheme 5) in 24% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=486.0; ¹H NMR (400 MHz, CD₃OD) δ 8.22 (d, 1H, J=2.0 Hz), 8.18 (d, 1H, J=2.0 Hz,), 7.37 (s, 1H, J=7.4 Hz), 7.1-7.03 (m, 3H), 6.9-6.86 (m, 1H), 4.3-4.27 (t, 2H, J=6.0 Hz), 4.15-4.12 (t, 2H, J=6.0 Hz), 3.99-3.89 (m, 2H), 2.1-1.98 (m, 4H), 1.83 (s, 3H).

### (S)-2-Amino-2-(5-(4-(4-(3-fluorophenoxy)butoxy)-3-(trifluoromethyl)phenyl)-1,3,4-thiadiazol-2-yl)propan-1-ol (8n)

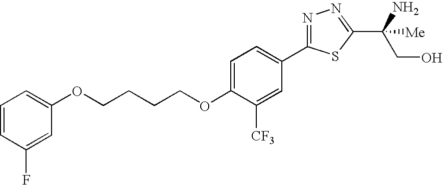

The title product was obtained according to general procedure (Scheme 5) in 54% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=486.0; ¹H NMR (400 MHz, CD₃OD) δ 8.22 (d, 1H, J=2.4 Hz), 8.18-8.16 (d, 1H J=2.0 Hz), 7.37 (s, 1H, J=7.4 Hz), 7.25-7.2 (m, 1H), 6.73-6.72 (d, 1H, J=8.4 Hz), 6.67-6.61 (m, 2H), 4.29 (t, 2H, J=5.6 Hz), 4.07 (t, 2H, J=6.0 Hz), 3.99-3.89 (m, 2H), 2.06-1.97 (m, 4H), 1.83 (s, 3H).

### (S)-2-Amino-2-(5-(3-(trifluoromethyl)-4-(4-(4-(trifluoromethyl)phenoxy)butoxy)phenyl)-1,3,4-thiadiazol-2-yl)propan-1-ol (8o)

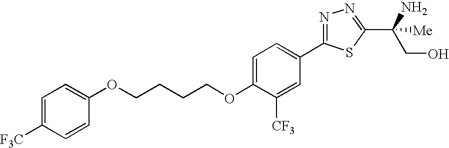

The title product was obtained according to general procedure (Scheme 5) in 32% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=535.9; ¹H NMR (400 MHz, CD₃OD) δ 8.21 (d, 1H, J=2.4 Hz), 8.18-8.16 (d, 1H J=2 Hz), 7.37 (s, 1H, J=7.4 Hz), 7.25-7.28 (m, 1H), 6.73-6.72 (d, 1H, J=8.4 Hz), 6.67-6.61 (m, 2H), 4.29 (t, 2H, J=5.6 Hz), 4.07 (t, 2H, J=6.0 Hz), 3.99-3.89 (m, 2H), 2.06-1.97 (m, 4H), 1.83 (s, 3H).

### (S)-2-Amino-2-(5-(3-(trifluoromethyl)-4-(4-(2-(trifluoromethyl)phenoxy)butoxy)phenyl)-1,3,4-thiadiazol-2-yl)propan-1-ol (8p)

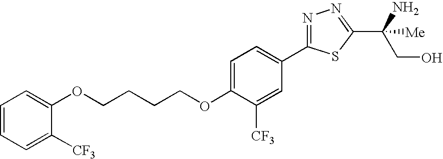

The title product was obtained according to general procedure (Scheme 5) in 32% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=536.0; ¹H NMR (400 MHz, CD₃OD) δ 8.2 (d, 1H), 8.16 (d, 1H J=8.8 Hz), 7.55 (d, 2H, J=7.6 Hz), 7.36 (d, 1H, J=7.4 Hz), 7.16 (d, 1H, J=8.4 Hz), 7.04 (d, 1H, J=7.2 Hz), 4.29 (t, 2H, J=5.4 Hz), 4.18 (t, 2H, J=5.4 Hz), 3.97-3.85 (m, 2H), 2.06 (m, 4H), 1.78 (s, 3H).

### (S)-2-Amino-2-(5-(3-(trifluoromethyl)-4-(4-(3-(trifluoromethyl)phenoxy)butoxy)phenyl)-1,3,4-thiadiazol-2-yl)propan-1-ol (8q)

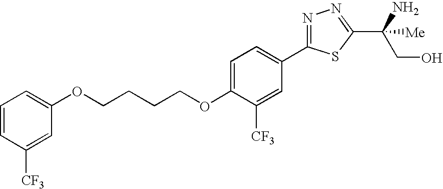

The title product was obtained according to general procedure (Scheme 5) in 47% yield from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=535.9; ¹H NMR (400 MHz, CD₃OD) δ 8.15-8.23 (m, 1H), 7.43 (t, 1H, J=7.6 Hz), 7.38 (d, 1H, J=7.4 Hz), 7.20-7.13 (m, 3H,), 4.3 (t, 2H, J=5.4 Hz), 4.13 (t, 2H, J=6.0 Hz), 3.99-3.89 (m, 2H), 2.07-2.04 (m, 4H), 1.83 (s, 3H).

### (S)-3-(5-(4-(5-(2-Amino-1-hydroxypropan-2-yl)-1,3,4-thiadiazol-2-yl)-2-(trifluoromethyl)phenoxy)pentyloxy)benzonitrile (8r)

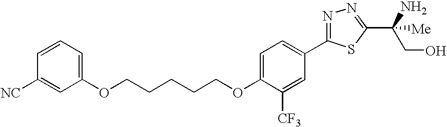

The title product was obtained according to general procedure (Scheme 5) in 33% yield from compound 9b. MS (ESI, M+H⁺)=507.1; ¹H NMR (400 MHz, CD₃OD) δ 8.21 (s, 1H,), 8.16 (d, 1H, J=8.8 Hz), 7.42 (t, 1H, J=7.6 Hz), 7.36 (d, 1H, J=8.8 Hz), 7.26-7.21 (m, 3H), 4.25 (t, 2H, J=6.0 Hz), 4.06 (t, 2H, J=5.6 Hz), 3.99-3.89 (m, 2H,), 1.92-1.85 (m, 4H), 1.83 (s, 3H), 1.76-1.70 (m, 2H).

### (S)-4-(5-(4-(5-(2-Amino-1-hydroxypropan-2-yl)-1,3,4-thiadiazol-2-yl)-2-(trifluoromethyl)phenoxy)pentyloxy)benzonitrile (8s)

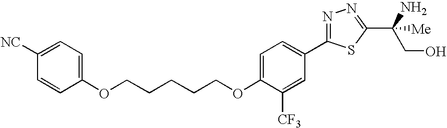

The title product was obtained according to general procedure (Scheme 5) in 21% yield from compound 9b. MS (ESI, M+H⁺)=507.1; ¹H NMR (400 MHz, CD₃OD) δ 8.21 (s, 1H,), 8.16 (d, 1H, J=8.8 Hz), 7.63-7.60 (m, 2H), 7.35 (d, 1H, J=8.8 Hz), 7.05-7.02 (m, 2H), 4.25 (t, 2H, J=6.0 Hz), 4.10 (t, 2H, J=5.6 Hz), 3.94 (m, 2H,), 2.65 (s, 2H), 1.95-1.86 (m, 4H), 1.83 (s, 3H), 1.76-1.70 (m, 2H).

### (S)-2-(5-(4-(5-(2-Amino-1-hydroxypropan-2-yl)-1,3,4-thiadiazol-2-yl)-2-(trifluoromethyl)phenoxy)pentyloxy)benzonitrile (8t)

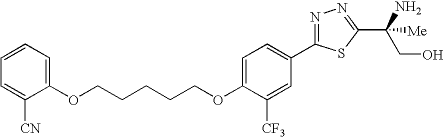

The title product was obtained according to general procedure (Scheme 5) in 28% yield from compound 9b. MS (ESI, M+H⁺)=507.1; ¹H NMR (400 MHz, CD₃OD) δ 8.21 (s, 1H,), 8.16 (d, 1H, J=9.2 Hz), 7.62-7.58 (m, 2H), 7.37 (d, 1H, J=8.8 Hz), 7.16 (d, 1H J=8.8 Hz), 7.04 (t, 1H, J=7.6 Hz) 4.26 (t, 2H, J=6.0 Hz), 4.18 (t, 2H, J=6.0 Hz), 1.96 (m, 4H,), 1.82-1.77 (m, 5H).

### (S)-2-Amino-2-(5-(3-(trifluoromethyl)-4-(5-(2-(trifluoromethyl)phenoxy)pentyloxy)phenyl)-1,3,4-thiadiazol-2-yl)propan-1-ol (8u)

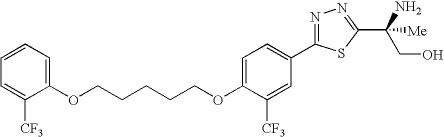

The title product was obtained according to general procedure (Scheme 5) in 40% yield from compound 9b. MS (ESI, M+H⁺)=550.0; ¹H NMR (400 MHz, CD₃OD) δ 8.22 (s, 1H,), 8.17 (d, 1H, J=9.6 Hz), 7.53 (m, 2H), 7.36 (d, 1H, J=9.2 Hz), 7.15 (d, 1H, J=7.6 Hz), 7.02 (t, 1H, J=7.2 Hz), 4.24 (t, 2H, J=6.0 Hz), 4.12 (t, 2H, J=5.6 Hz), 3.99-3.90 (m, 2H,), 1.94-1.75 (m, 9H).

### (S)-2-Amino-2-(5-(3-(trifluoromethyl)-4-(5-(4-(trifluoromethyl)phenoxy)pentyloxy)phenyl)-1,3,4-thiadiazol-2-yl)propan-1-ol (8v)

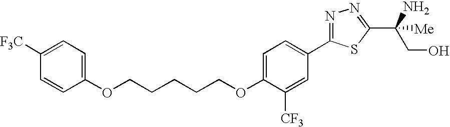

The title product was obtained according to general procedure (Scheme 5) in 53% yield from compound 9b. MS (ESI, M+H⁺)=550.0; ¹H NMR (400 MHz, CD₃OD) δ 8.21 (s, 1H,), 8.17 (d, 1H, J=8.8 Hz), 7.55 (d, 2H, J=8.4 Hz), 7.36 (d, 1H, J=8.4 Hz), 7.04 (d, 2H, J=8.0 Hz), 4.25 (t, 2H, J=6.0 Hz), 4.08 (t, 2H, J=6.4 Hz), 3.99-3.89 (m, 2H,), 1.96-1.73 (m, 9H).

### (S)-2-Amino-2-(5-(4-(5-(2,6-difluorophenoxy)pentyloxy)-3-(trifluoromethyl)phenyl)-1,3,4-thiadiazol-2-yl)propan-1-ol (8w)

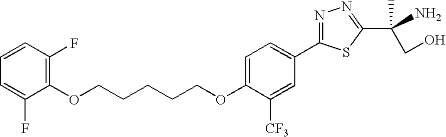

The title compound was prepared from protected phenyl-thiadiazole 6a in 42% (15.6 mg) yield. MS (ESI, M+H⁺)=518.0; ¹H NMR (400 MHz, CD₃OD) δ 8.17 (d, 1H, J=2.0 Hz), 8.12 (dd, 1H, J=8.8 Hz, J=2.4 Hz), 7.32 (d, 1H, J=8.4 Hz), 6.92-7.04 (m, 3H), 4.22 (t, 2H, J=5.6 Hz), 4.14 (t, 2H, J=6.4 Hz), 3.70-3.87 (m, 2H), 1.95-1.71 (m, 6H), 1.60 (s, 3H).

### (S)-2-Amino-2-(5-(4-(5-(3,5-difluorophenoxy)pentyloxy)-3-(trifluoromethyl)phenyl)-1,3,4-thiadiazol-2-yl)propan-1-ol (8x)

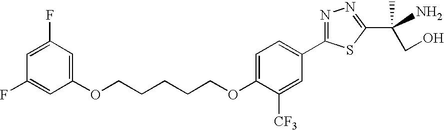

The title compound was prepared from protected phenyl-thiadiazole 6a in 38% (14.1 mg) yield. MS (ESI, M+H⁺)=518.1; ¹H NMR (400 MHz, CD₃OD) δ 8.21 (s, 1H), 8.17 (dd, 1H, J=8.4 Hz, J=2.4 Hz), 7.36 (d, 1H, J=8.4 Hz), 6.52-6.43 (m, 3H), 4.24 (t, 2H, J=5.6 Hz), 4.02-3.89 (m, 4H), 1.95-1.83 (m, 4H), 1.82 (s, 3H), 1.74-1.69 (m, 2H),

### (S)-2-Amino-2-(5-(4-(5-(2,5-difluorophenoxy)pentyloxy)-3-(trifluoromethyl)phenyl)-1,3,4-thiadiazol-2-yl)propan-1-ol (8y)

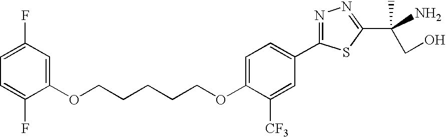

The title compound was prepared from protected phenyl-thiadiazole 6a in 36% (13.4 mg) yield. MS (ESI, M+H⁺)=518.0; ¹H NMR (400 MHz, CD₃OD) δ 8.21 (d, 1H, J=2.4 Hz), 8.16 (dd, 1H, J=8.8 Hz, J=2.0 Hz), 7.36 (d, 1H, J=8.4 Hz), 7.08-7.01 (m, 1H), 6.89-6.84 (m, 1H), 6.62-6.56 (m, 1H), 4.25 (t, 2H, J=6.4 Hz), 4.06 (t, 2H, J=6.4 Hz), 3.99-3.89 (m, 2H), 1.98-1.86 (m, 4H), 1.83 (s, 3H), 1.76-1.69 (m, 2H),

### (S)-2-Amino-2-(5-(4-(5-(2,3-difluorophenoxy)pentyloxy)-3-(trifluoromethyl)phenyl)-1,3,4-thiadiazol-2-yl)propan-1-ol (8z)

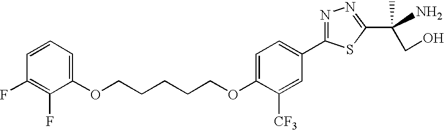

The title compound was prepared from protected phenyl-thiadiazole 6a in 54% (20 mg) yield. MS (ESI, M+H⁺)=518.0; ¹H NMR (400 MHz, CD₃OD) δ 8.21 (s, 1H), 8.16 (dd, 1H, J=8.8 Hz, J=2.0 Hz), 7.35 (d, 1H, J=8.8 Hz), 7.05-6.99 (m, 1H), 6.90-6.86 (m, 1H), 6.82-6.75 (m, 1H),), 4.24 (t, 2H, J=6.4 Hz), 4.10 (t, 2H, J=6.0 Hz), 4.00-3.90 (m, 2H), 1.98-1.87 (m, 4H), 1.83 (s, 3H), 1.77-1.69 (m, 2H),

### (S)-2-Amino-2-(5-(4-(5-phenoxypentyloxy)-3-(trifluoromethyl)phenyl)-1,3,4-thiadiazol-2-yl)propan-1-ol (8aa)

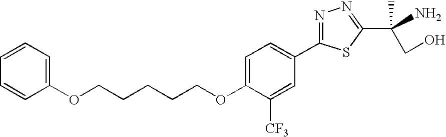

The title compound was prepared from protected phenyl-thiadiazole 6a in 35% yield. MS (ESI, M+H⁺)=482.1; ¹H NMR (400 MHz, CD₃OD) δ 8.21 (d, 1H, J=2.4 Hz), 8.16 (dd, 1H, J=8.8 Hz, J=2.0 Hz), 7.36 (d, 1H, J=8.8 Hz), 7.25-7.20 (m, 2H), 6.90-6.86 (m, 3H), 4.24 (t, 2H, J=5.6 Hz), 4.01-3.90 (m, 4H), 1.95-1.83 (m, 7H), 1.75-1.69 (m, 2H).

### (S)-2-Amino-2-(5-(4-(5-phenoxypentyloxy)-3-(trifluoromethyl)phenyl)-1,3,4-thiadiazol-2-yl)propan-1-ol (8ab)

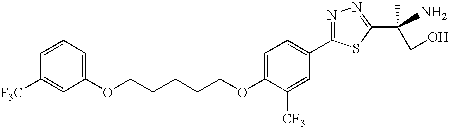

The title compound was prepared from protected phenyl-thiadiazole 6a in 40% (29.1 mg) yield. MS (ESI, M+H⁺)=550.0; ¹H NMR (400 MHz, CD₃OD) δ 8.21 (d, 1H, J=2.4 Hz), 8.17 (dd, 1H, J=8.8 Hz, J=2.4 Hz), 7.43 (t, 1H, J=7.6 Hz), 7.36 (d, 1H, J=8.8 Hz), 7.20-7.14 (m, 3H), 4.25 (t, 2H, J=6.4 Hz), 4.07 (t, 2H, J=6.0 Hz), 3.99-3.89 (m, 2H), 1.98-1.86 (m, 4H), 1.83 (s, 3H), 1.77-1.70 (m, 2H).

### (S)-2-Amino-2-(5-(4-(5-(2-fluorophenoxy)pentyloxy)-3-(trifluoromethyl)phenyl)-1,3,4-thiadiazol-2-yl)propan-1-ol (8ac)

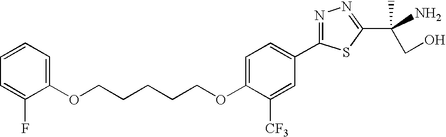

The title compound was prepared from protected phenyl-thiadiazole 6a in 38% (27.2 mg) yield. MS (ESI, M+H⁺)=500.1; ¹H NMR (400 MHz, CD₃OD) δ 8.21 (d, 1H, J=2.0 Hz), 8.16 (dd, 1H, J=8.4 Hz, J=2.4 Hz), 7.35 (d, 1H, J=8.4 Hz), 7.07-7.02 (m, 3H), 6.90-6.85 (m, 1H), 4.24 (t, 2H, J=6.0 Hz), 4.07 (t, 2H, J=6.0 Hz), 3.99-3.90 (m, 2H), 1.97-1.83 (m, 7H), 1.76-1.69 (m, 2H).

### (S)-2-Amino-2-(5-(4-(5-(3-fluorophenoxy)pentyloxy)-3-(trifluoromethyl)phenyl)-1,3,4-thiadiazol-2-yl)propan-1-ol (8ad)

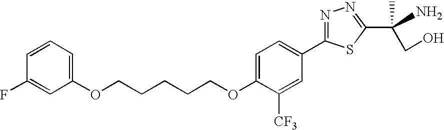

The title compound was prepared from protected phenyl-thiadiazole 6a in 49% (35.2 mg) yield. MS (ESI, M+H⁺)=500.0; ¹H NMR (400 MHz, CD₃OD) δ 8.21 (s, 1H), 8.16 (d, 1H, J=9.2 Hz), 7.36 (d, 1H, J=8.8 Hz), 7.25-7.21 (m, 1H), 6.72-6.60 (m, 3H), 4.24 (t, 2H, J=5.6 Hz), 4.02-3.88 (m, 4H), 1.95-1.82 (m, 7H), 1.73 (m, 2H).

### (S)-2-Amino-2-(5-(4-(5-(4-fluorophenoxy)pentyloxy)-3-(trifluoromethyl)phenyl)-1,3,4-thiadiazol-2-yl)propan-1-ol (8ae)

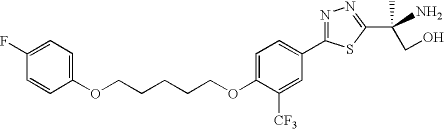

The title compound was prepared from protected phenyl-thiadiazole 6a in 13% (9.2 mg) yield. MS (ESI, M+H⁺)=500.0; ¹H NMR (400 MHz, CD₃OD) δ 8.21 (s, 1H), 8.16 (d, 1H, J=8.4 Hz), 7.36 (d, 1H, J=8.8 Hz), 6.96 (m, 2H), 6.88-6.85 (m, 2H), 4.24 (t, 2H, J=6.4 Hz), 3.99-3.90 (m, 4H), 1.95-1.82 (m, 7H), 1.74-1.71 (m, 2H).

### (S)-2-(5-(4-(4-(2,3-Difluorophenoxy)butoxy)-3-(trifluoromethyl)phenyl)-1,3,4-thiadiazol-2-yl)-2-aminopropan-1-ol (8af)

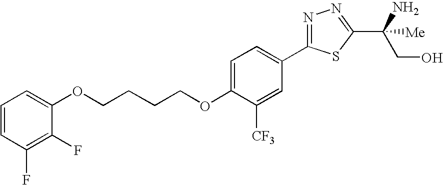

The title product was obtained according to general procedure (Scheme 5) from compound 9a. HPLC retention time on a Synergi MAX-RP 100A (20×2 mm, 2μ) was 1.58 min with gradient 20-95% acetonitrile-H₂O (0.1% TFA) in 2.0 min as mobile phase. MS (ESI, M+H⁺)=504.1; ¹H NMR (400 MHz, CD₃OD) δ 8.22 (d, 1H, J=2.4 Hz), 8.19-8.16 (d,d 1H J=2 Hz), 7.37 (d, 1H, J=8.4 Hz), 7.07-7 (m, 1H), 6.9-6.88 (m, 1H), 6.8-6.76 (m, 1H), 4.3 (t, 2H, J=11.2 Hz), 4.17 (t, 2H, J=11.2 Hz), 3.99-3.89 (m, 2H), 2.07-2.04 (m, 4H), 1.83 (s, 3H).

### (S)-2-(5-(4-(4-(2,4-Difluorophenoxy)butoxy)-3-(trifluoromethyl)phenyl)-1,3,4-thiadiazol-2-yl)-2-aminopropan-1-ol (8ag)

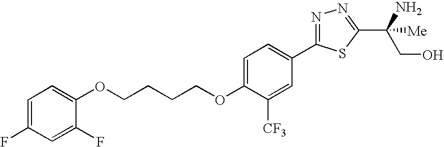

The title product was obtained according to general procedure (Scheme 5) from compound 9a. HPLC retention time on a Synergi MAX-RP 100A (20×2 mm, 2μ) was 1.57 min with gradient 20-95% acetonitrile-H₂O (0.1% TFA) in 2.0 min as mobile phase. MS (ESI, M+H⁺)=504.0; ¹H NMR (400 MHz, CD₃OD) δ 8.21 (d, 1H, J=2 Hz), 8.18-8.16 (d,d 1H J=2 Hz), 7.37 (d, 1H, J=8.8 Hz), 7.10-7.05 (m, 1H, J=24 Hz), 6.96-6.9 (m, 1H), 6.87-6.81 (m, 1H), 4.3 (t, 2H, J=11.2 Hz), 4.11 (t, 2H, J=12 Hz), 3.99-3.89 (m, 2H), 2.09-1.97 (m, 4H), 1.83 (s, 3H).

### (S)-2-(5-(4-(4-(2,5-Difluorophenoxy)butoxy)-3-(trifluoromethyl)phenyl)-1,3,4-thiadiazol-2-yl)-2-aminopropan-1-ol (8ah)

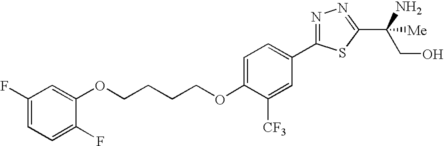

The title product was obtained according to general procedure (Scheme 5) from compound 9a. HPLC retention time on a Synergi MAX-RP 100A (20×2 mm, 2 g) was 1.57 min with gradient 20-95% acetonitrile-H₂O (0.1% TFA) in 2.0 min as mobile phase. MS (ESI, M+H⁺)=504.0; ¹H NMR (400 MHz, CD₃OD) δ 8.22 (d, 1H, J=2 Hz), 8.19-8.16 (d,d 1H J=2 Hz), 7.37 (d, 1H, J=8.4 Hz), 7.08-7.02 (m, 1H), 6.64-6.58 (m, 1H), 6.87-6.81 (m, 1H), 4.3 (t, 2H, J=10.8 Hz), 4.13 (t, 2H, J=11.6 Hz), 3.99-3.89 (m, 2H), 2.07-2.02 (m, 4H), 1.83 (s, 3H).

### (S)-2-(5-(4-(4-(2,6-Difluorophenoxy)butoxy)-3-(trifluoromethyl)phenyl)-1,3,4-thiadiazol-2-yl)-2-aminopropan-1-ol (8ai)

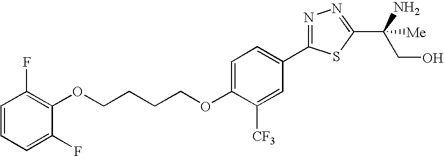

The title product was obtained according to general procedure (Scheme 5) from compound 9a. HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=503.9; ¹H NMR (400 MHz, CD₃OD) δ 8.21 (d, 1H, J=2.4 Hz), 8.19-8.16 (d,d 1H J=2 Hz), 7.37 (d, 1H, J=8.8 Hz), 7.07-6.92 (m, 3H), 4.3 (t, 2H, J=12 Hz), 4.2 (t, 2H, J=12.4 Hz), 3.98-3.87 (m, 2H), 2.11-1.93 (m, 4H), 1.83 (s, 3H).

### (S)-2-(5-(4-(4-(3,5-Difluorophenoxy)butoxy)-3-(trifluoromethyl)phenyl)-1,3,4-thiadiazol-2-yl)-2-aminopropan-1-ol (8aj)

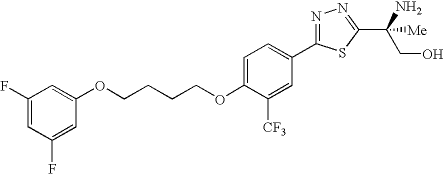

The title product was obtained according to general procedure (Scheme 5) from compound 9a. HPLC retention time on a Synergi MAX-RP 100A (20×2 mm, 2μ) was 1.60 min with gradient 20-95% acetonitrile-H₂O (0.1% TFA) in 2.0 min as mobile phase. MS (ESI, M+H⁺)=503.9; ¹H NMR (400 MHz, CD₃OD) δ 8.21 (d, 1H, J=2 Hz), 8.19-8.16 (d,d 1H J=2 Hz), 7.37 (d, 1H, J=8.8 Hz), 6.55-6.44 (m, 3H), 4.3 (t, 2H, J=11.2 Hz), 4.07 (t, 2H, J=12 Hz), 3.97-3.86 (m, 2H), 2.04-1.99 (m, 4H), 1.8 (s, 3H).

### (S)-2-Amino-2-(5-(4-(4-(4-chlorophenoxy)butoxy)-3-(trifluoromethyl)phenyl)-1,3,4-thiadiazol-2-yl)propan-1-ol (8ak)

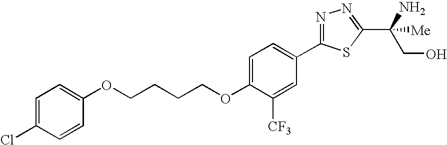

HPLC retention time on a Synergi MAX-RP 100A (20×2 mm, 2μ) was 1.63 min with gradient 20-95% acetonitrile-H₂O (0.1% TFA) in 2.0 min as mobile phase. MS (ESI, M+H⁺)=501.8; ¹H NMR (400 MHz, CD₃OD) δ 8.22 (d, 1H, J=2.4 Hz), 8.19-8.16 (dd, 1H, J=8.8 Hz, J=2.4 Hz), 7.37 (d, 1H, J=8.4 Hz), 7.22 (d, 2H, J=8.8 Hz), 6.88 (d, 2H, J=9.0 Hz), 4.30 (t, 2H, J=5.6 Hz), 4.17 (t, 2H, J=6.0 Hz), 3.97 (AB, 1H, J_{AB}=11.6 Hz), 3.92 (AB, 1H, J_{AB}=11.6 Hz), 2.07-2.04 (m, 4H), 1.83 (s, 3H).

### (S)-2-Amino-2-(5-(4-(4-(3-chlorophenoxy)butoxy)-3-(trifluoromethyl)phenyl)-1,3,4-thiadiazol-2-yl)propan-1-ol (8al)

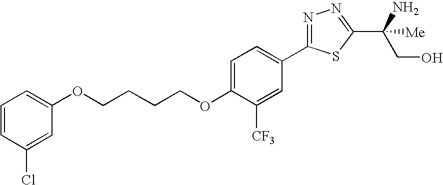

HPLC retention time on a Synergi MAX-RP 100A (20×2 mm, 2μ) was 1.64 min with gradient 20-95% acetonitrile-H₂O (0.1% TFA) in 2.0 min as mobile phase. MS (ESI, M+H⁺)=501.9; ¹H NMR (400 MHz, CD₃OD) δ 8.22 (d, 1H, J=2.0 Hz), 8.18 (dd, 1H, J=8.8 Hz, J=2.4 Hz), 7.37 (d, 1H, J=8.8 Hz), 7.23-7.19 (m, 1H), 6.91-6.83 (m, 3H), 4.3 (t, 2H, J=5.6 Hz), 4.07 (t, 2H, J=6.0 Hz), 3.98 (AB, 1H, J_{AB}=11.6 Hz), 3.91 (AB, 1H, J_{AB}=11.6 Hz), 2.06-1.98 (m, 4H), 1.83 (s, 3H).

### (S)-2-Amino-2-(5-(4-(4-(2-chlorophenoxy)butoxy)-3-(trifluoromethyl)phenyl)-1,3,4-thiadiazol-2-yl)propan-1-ol (8am)

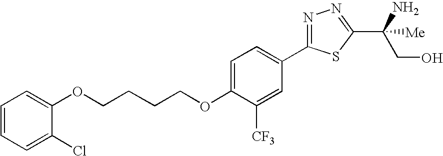

HPLC retention time on a Synergi MAX-RP 100A (20×2 mm, 2 g) was 1.59 min with gradient 20-95% acetonitrile-H₂O (0.1% TFA) in 2.0 min as mobile phase. MS (ESI, M+H⁺)=501.8; ¹H NMR (400 MHz, CD₃OD) δ 8.21 (d, 1H, J=2.4 Hz), 8.17 (dd, 1H, J=8.8 Hz, J=2.4 Hz), 7.38 (d, 1H, J=8.4 Hz), 7.33-7.31 (m, 1H), 7.25-7.21 (m, 1H), 7.05 (dd, 1H, J=8.4 Hz, J=1.2 Hz), 6.91-6.87 (m, 1H), 4.33 (t, 2H, J=5.8 Hz), 4.14 (t, 2H, J=6.0 Hz), 3.97 (AB, 1H, J_{AB}=11.6 Hz), 3.9 (AB, 1H, J_{AB}=11.6 Hz), 2.12-2.04 (m, 4H), 1.83 (s, 3H).

### (S)-2-(4-(4-(5-(2-Amino-1-hydroxypropan-2-yl)-1,3,4-thiadiazol-2-yl)-2-(trifluoromethyl)phenoxy)butoxy)benzonitrile (8an)

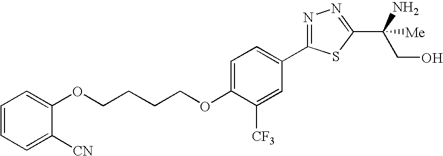

HPLC retention time on a Synergi MAX-RP 100A (20×2 mm, 2μ) was 1.48 min with gradient 20-95% acetonitrile-H₂O (0.1% TFA) in 2.0 min as mobile phase. MS (ESI, M+H⁺)=493.0; ¹H NMR (400 MHz, CD₃OD) δ 8.21 (d, 1H, J=2.0 Hz), 8.17 (dd, 1H, J=8.8 Hz, J=2.4 Hz), 7.57-7.63 (m, 2H), 7.41 (d, 1H, J=8.4 Hz), 7.17 (d, 1H, J=8.4 Hz), 7.07-7.03 (m, 1H), 4.34 (t, 2H, J=5.6 Hz), 4.24 (t, 2H, J=6.0 Hz), 3.98 (AB, 1H, J_{AB}=11.2 Hz), 3.9 (AB, 1H, J_{AB}=11.6 Hz), 2.13-2.06 (m, 4H), 1.83 (s, 3H).

### (S)-3-(4-(4-(5-(2-Amino-1-hydroxypropan-2-yl)-1,3,4-thiadiazol-2-yl)-2-(trifluoromethyl)phenoxy)butoxy)benzonitrile (8ao)

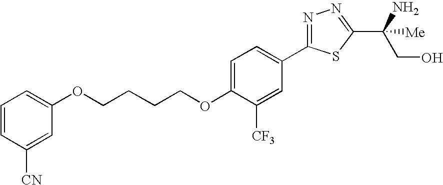

HPLC retention time on a Synergi MAX-RP 100A (20×2 mm, 2μ) was 1.48 min with gradient 20-95% acetonitrile-H₂O (0.1% TFA) in 2.0 min as mobile phase. MS (ESI, M+H⁺)=493.0; ¹H NMR (400 MHz, CD₃OD) δ 8.21 (d, 1H, J=2.0 Hz), 8.19-8.16 (m, 1H), 7.64-7.62 (m, 2H), 7.37 (d, 1H, J=8.8 Hz), 7.07 (m, 2H), 4.32 (t, 2H, J=5.6 Hz), 4.16 (t, 2H, J=6.0 Hz), 3.98 (AB, 1H, J_{AB}=11.6 Hz), 3.91 (AB, 1H, J_{AB}=11.6 Hz), 2.05-2.04 (m, 4H), 1.83 (s, 3H).

### (S)-4-(4-(4-(5-(2-Amino-1-hydroxypropan-2-yl)-1,3,4-thiadiazol-2-yl)-2-(trifluoromethyl)phenoxy)butoxy)benzonitrile (8ap)

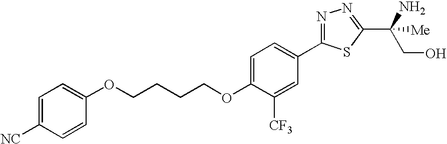

HPLC retention time on a Synergi MAX-RP 100A (20×2 mm, 2 g) was 1.50 min with gradient 20-95% acetonitrile-H₂O (0.1% TFA) in 2.0 min as mobile phase. MS (ESI, M+H⁺)=493.0; ¹H NMR (400 MHz, CD₃OD) δ 8.21 (d, 1H, J=2.0 Hz), 8.19-8.16 (m, 1H), 7.44-7.36 (m, 2H), 7.27 (m, 3H), 4.28 (t, 2H, J=5.6 Hz), 4.12 (t, 2H, J=6.0 Hz), 3.98 (AB, 1H, J_{AB}=11.6 Hz), 3.92 (AB, 1H, J_{AB}=11.2 Hz), 2.07-2.02 (m, 4H), 1.83 (s, 3H).

### (S)-2-Amino-2-(5-(4-(6-ethoxyhexyloxy)-3-(trifluoromethyl)phenyl)-1,3,4-thiadiazol-2-yl)propan-1-ol (8aq)

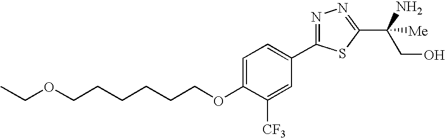

The title product was obtained according to general procedure (Scheme 5) in 20% yield from compound 9b. MS (ESI, M+H⁺)=448.1; ¹H NMR (400 MHz, CD₃OD)) δ 8.21 (s, 1H,), 8.17 (dd, 1H, J=8.8 Hz, J=2.0 Hz), 7.36 (d, 1H, J=8.8 Hz), 4.21 (t, 2H, J=6.0 Hz), 3.99-3.89 (m, 2H), 3.51-3.44 (m, 4H), 1.84-1.88 (m, 2H), 1.82 (s, 3H), 1.63-1.52 (m, 4H), 1.49-1.43 (m, 2H), 1.18 (t, 3H, J=6.8 Hz).

### (S)-2-Amino-2-(5-(4-(6-(benzyloxy)hexyloxy)-3-(trifluoromethyl)phenyl)-1,3,4-thiadiazol-2-yl)propan-1-ol (8ar)

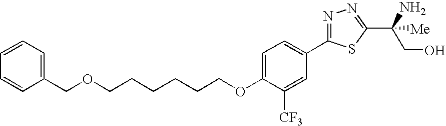

The title product was obtained according to general procedure (Scheme 5) in 11% yield from compound 9b. MS (ESI, M+H⁺)=510.1; ¹H NMR (400 MHz, CD₃OD) δ 8.21 (s, 1H,), 8.16 (dd, 1H, J=8.6 Hz, J=2.0 Hz), 7.35-7.24 (m, 6H), 4.49 (s, 2H), 4.20 (t, 2H, J=6.0 Hz), 3.99-3.89 (m, 2H,), 3.51 (t, 2H, J=6.4 Hz), 1.87-1.84 (m, 2H), 1.83 (s, 3H), 1.69-1.62 (m, 2H), 1.59-1.45 (m, 4H).

### (S)-2-Amino-2-(5-(4-(6-propoxyhexyloxy)-3-(trifluoromethyl)phenyl)-1,3,4-thiadiazol-2-yl)propan-1-ol (8as)

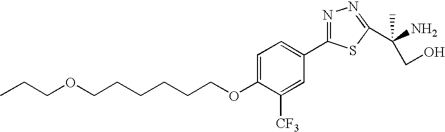

The title compound was prepared from protected phenyl-thiadiazole 6a in 18% (9.7 mg) yield. MS (ESI, M+H⁺)=462.1; ¹H NMR (400 MHz, CD₃OD) δ 8.21 (s, 1H), 8.17 (dd, 1H, J=8.8 Hz, J=2.4 Hz), 7.35 (d, 1H, J=8.8 Hz), 4.21 (t, 2H, J=5.6 Hz), 3.99-3.89 (m, 2H), 3.45 (t, 2H, J=6.4 Hz), 3.39 (t, 2H, J=6.8 Hz), 1.90-1.82 (m, 5H), 1.65-1.43 (m, 6H), 0.92 (t, 3H, J=7.6 Hz).

### (S)-2-Amino-2-(5-(4-(5-ethoxypentyloxy)-3-(trifluoromethyl)phenyl)-1,3,4-thiadiazol-2-yl)propan-1-ol (8at)

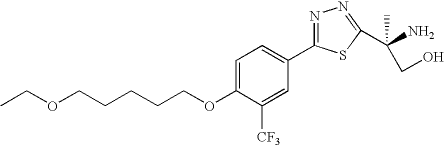

The title compound was prepared from protected phenyl-thiadiazole 6a in 35% (24.7 mg) yield. MS (ESI, M+H⁺)=434.0; ¹H NMR (400 MHz, CD₃OD) δ 8.21-8.16 (m, 2H), 7.36 (m, 1H), 4.21 (m, 2H), 3.99-3.89 (m, 2H), 3.48 (m, 4H), 1.87-1.83 (m, 5H), 1.65 (m, 4H), 1.17 (m, 3H).

### (S)-2-Amino-2-(5-(4-(5-propoxypentyloxy)-3-(trifluoromethyl)phenyl)-1,3,4-thiadiazol-2-yl)propan-1-ol (8au)

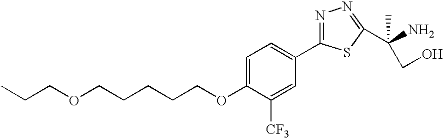

The title compound was prepared from protected phenyl-thiadiazole 6a in 36% (26.2 mg) yield. MS (ESI, M+H⁺)=448.0; ¹H NMR (400 MHz, CD₃OD) δ 8.21-8.16 (m, 2H), 7.36 (m, 1H), 4.21 (m, 2H), 3.99-3.90 (m, 2H), 3.46-3.37 (m, 4H), 1.88-1.83 (m, 5H), 1.65-1.56 (m, 6H), 0.92 (t, 3H, J=7.2 Hz).

### (S)-2-Amino-2-(5-(4-(5-(benzyloxy)pentyloxy)-3-(trifluoromethyl)phenyl)-1,3,4-thiadiazol-2-yl)propan-1-ol (8av)

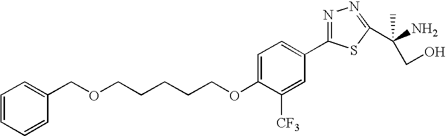

The title compound was prepared from protected phenyl-thiadiazole 6a in 32% (24.3 mg) yield. MS (ESI, M+H⁺)=496.1; ¹H NMR (400 MHz, CD₃OD) δ 8.21-8.14 (m, 2H), 7.35-7.26 (m, 6H), 4.49 (s, 2H), 4.20 (t, 2H, J=5.2 Hz), 3.99-3.90 (m, 2H), 3.53 (t, 2H, J=6.0 Hz), 1.86-1.83 (m, 5H), 1.69-1.61 (m, 4H).

### (S)-2-Amino-2-(5-(4-(4-propoxybutoxy)-3-(trifluoromethyl)phenyl)-1,3,4-thiadiazol-2-yl)propan-1-ol (8aw)

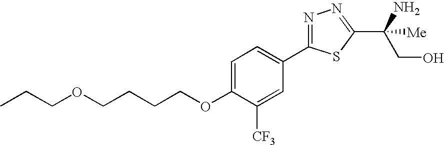

The title compound was prepared from protected phenyl-thiadiazole 9a in 37% (20 mg) yield. HPLC retention time on a C8(2) column (30×50 mm, 3 μL) is 1.82 min with gradient 30-98% acetonitrile-H₂O (0.1% TFA) in 3.5 min as mobile phase. MS (ESI, M+H⁺)=434.01; ¹H NMR (400 MHz, CD₃OD) δ 8.21 (d, 1H, J=2.0 Hz), 8.16 (dd, 1H, J=8.4 Hz, J=2.0 Hz), 7.35 (d, 1H, J=8.4 Hz), 4.24 (t, 2H, J=6.0 Hz), 3.97 (d, 1H, J=11.6 Hz); 3.90 (d, 1H, J=11.6 Hz); 3.52 (t, 2H, J=6.4 Hz), 3.41 (t, 2H, J=6.8 Hz), 1.92 (m, 2H), 1.83 (s, 3H), 1.77 (m, 2H), 1.58 (m, 2H), 0.92 (t, 3H, J=7.2 Hz).

### (S)-2-amino-2-(5-(4-(4-(benzyloxy)butoxy)-3-(trifluoromethyl)phenyl)-1,3,4-thiadiazol-2-yl)propan-1-ol (8ax)

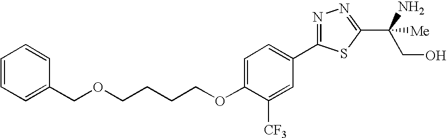

The title compound was prepared from protected phenyl-thiadiazole 9a in 77% (45 mg) yield. HPLC retention time on a C8(2) column (30×50 mm, 3 μL) is 1.92 min with gradient 30-98% acetonitrile-H₂O (0.1% TFA) in 3.5 min as mobile phase. MS (ESI, M+H⁺)=482.01; ¹H NMR (400 MHz, CD₃OD) δ 8.21 (d, 1H, J=2.0 Hz), 8.14 (dd, 1H, J=8.4 Hz, J=2.0 Hz), 7.32 (m, 5H), 7.26 (m, 1H), 4.51 (s, 2H), 4.22 (t, 2H, J=6.4 Hz), 3.98 (d, 1H, J=11.2 Hz); 3.91 (d, 1H, J=11.2 Hz); 3.58 (t, 2H, J=6.0 Hz), 1.94 (m, 2H), 1.84 (m, 2H), 1.83 (s, 3H).
General Protocol for Synthesis of 2-amino-acetophenone 3 (See Scheme 1)

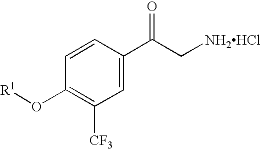

To a solution of the substituted acetophenone 2 (1.0 equiv) in dry CH₂Cl₂ under nitrogen atmosphere was added Bu₄NBr₃ (0.60 g, 1.0 equiv). To the solution was added anhydrous MeOH (10% v/v). The reaction mixture was stirred at RT 3-16 hours. The solvent removed in vacuo.

To a mixture of the desired bromo-acetophenone (from last step, 1.0 equiv), in DMF was added NaN₃ (3.0 equiv), then stirred in DMF for 1 hour. The reaction mixture was diluted with EtOAc and washed with H₂O (2×50). The solvent removed in vacuo and the product was purified by silica gel column chromatography using the Combi-Flash system (Hex:EtOAc).

To a solution of the azido-acetophenone (1.0 equiv) in MeOH was added concentrated HCl (3.0-0.50 equiv), and 10% Pd/C (10% w). The reaction mixture was stirred under an atmosphere of H₂ (g) for 1-4 hours. The reaction was then filtered through a thin layer of celite and the solvent removed in vacuo to afford the amino-acetophenone 3
General Protocol for Synthesis of Compound 6 (See Scheme 1)

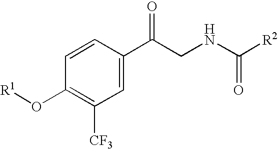

To a solution of desired carboxylic acid 4 or 5 (1 equiv), HATU (1.2 equiv), and DIEA (10 equiv) in DCM/DMF (1:1, 10 mL) was added amino-acetophenone 3 (1.0 equiv). The resultant mixture was stirred at RT for 3-16 hours. The reaction mixture was diluted with EtOAc and washed with 10% NH₄Cl (2×) and saturated NaCl (1×). The solvent removed in vacuo and the product was purified by silica gel column chromatography using Combi-Flash system (Hex:EtOAc).

### (S)-t-Butyl 2,2,4-trimethyl-4-(2-oxo-2-(4-(2-(pentyloxy)ethoxy)-3-(trifluoromethyl)phenyl)ethylcarbamoyl)oxazolidine-3-carboxylate (6a)

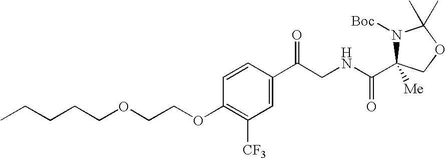

The title product was obtained according to general procedure (Scheme 1) from compound 3a. MS (ESI, M+H⁺)=574.0; ¹H NMR (400 MHz, CDCl₃) δ 8.22 (d, 1H, J=2.0 Hz), 8.12 (d, 1H, J=8.4 Hz), 7.12 (d, 1H, J=8.4 Hz), 4.71 (dd, 2H, J=6.0 Hz, J=4.0 Hz), 4.30 (m, 3H), 3.83 (m, 3H), 3.54 (m, 2H), 1.77 (s, 3H), 1.66 (s, 3H), 1.61-1.55 (m, 5H), 1.48-1.46 (m, 9H), 1.34-1.29 (m, 4H), 0.87 (m, 3H).
General Protocol for Synthesis of phenyl-thiazole 7 (See Scheme 1)

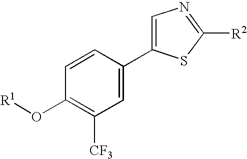

A suspension of protected amide 6 (1.0 equiv) and Lawesson's Reagent (1.0-1.2 equiv) in toluene was heated at 85-100° C. for 1-3 hours. After cooling to RT, the reaction mixture was purified by silica gel column chromatography using the Combi-Flash system (Hex:EtOAc).
General Protocol for One Pot Deprotection of Both Boc and Oxazolidine 8 (See Scheme 1)

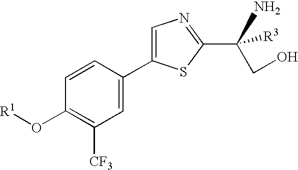

Refer to general protocol for one pot deprotection of both Boc and oxazolidine (synthesis of 8) in scheme 4.

### (S)-2-Amino-2-(5-(4-(2-(pentyloxy)ethoxy)-3-(trifluoromethyl)phenyl)thiazol-2-yl)propan-1-ol (8a)

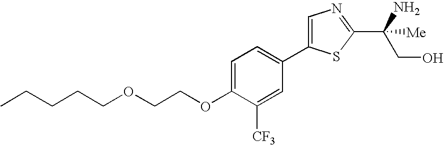

The title product was obtained according to general procedure (Scheme 1) from compound 7a. MS (ESI, M+H⁺)=432.9; ¹H NMR (400 MHz, CD₃OD) δ 8.04 (s, 1H), 7.82 (dd, 1H, J=8.4 Hz, J=2.4 Hz), 7.30 (d, 1H, J=8.4 Hz), 4.28 (dd, 2H, J=6.0 Hz, J=4.8 Hz), 3.94, 3.85 (AB, 2H, J=11.6 Hz), 3.82 (m, 2H), 3.55 (t, 2H, J=5.6 Hz), 1.78 (s, 3H), 1.58 (m, 2H), 1.34 (m, 4H), 0.89 (m, 3H).

General Approach to Synthesis of ether-phenyl-thiazoles

The synthesis of 2,5-substituted thiazoles is described in Scheme 6. Reaction of the desired alcohol para-methoxybenzyl alcohol (PMB-OH) with substituted 4-fluoroacetophenone 1 afforded the acetophenone intermediate 2. Acetophenone intermediate 2 was then converted to the corresponding bromo-acetophenone using Bu₄NBr₃ which, upon reaction with NaN₃, provided the azido-acetophenone intermediate. Hydrogenation of the azido-acetophenone intermediate afforded amine 3, followed by coupling with orthogonally protected amino acid 4 gave amide 5. Removal of PMB group under hydrogenation gave phenol 6. Mitsunobu reaction of the phenol 6 with the desired alcohol followed by thiazole formation under Lawesson's reagent conditions afforded intermediate 7 in good yield. Removal of the protecting group from intermediate 7 afforded the final amino-alcohol 8.

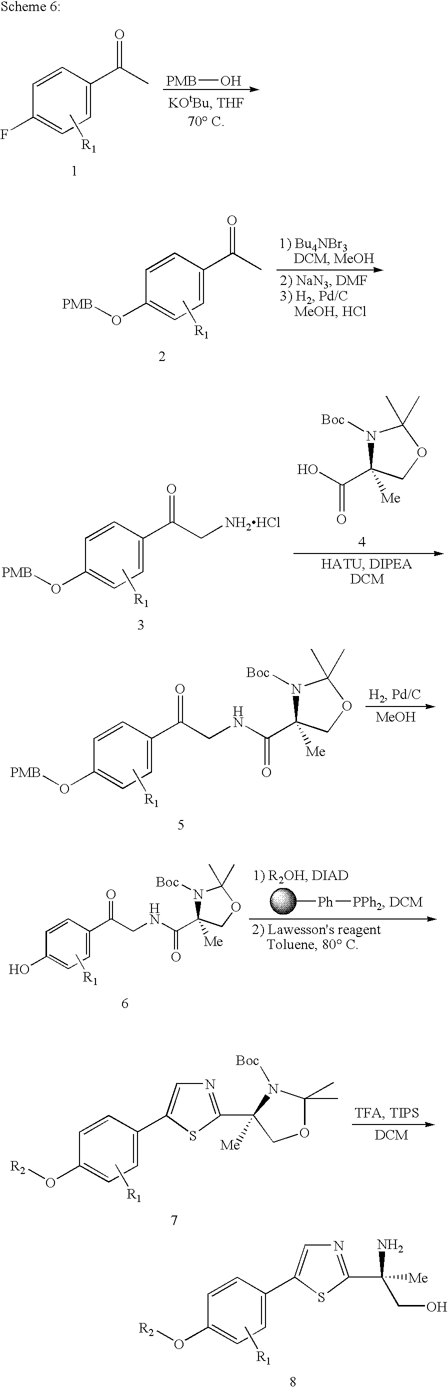

### 1-(4-(4-Methoxybenzyloxy)-3-(trifluoromethyl)phenyl)ethanone (2a)

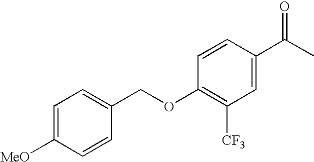

The title product was obtained according to general procedure (Scheme 1). The product was purified by silica gel column chromatography using the Combi-Flash system (Hex:EtOAc) as colorless oil in 90% (4.25 g). HPLC retention time on a C8(2) column (30×3.00 mm, 3μ) is 2.02 min with gradient 50-98% acetonitrile-H₂O (0.1% TFA) in 4.0 min as mobile phase. TLC (1:3 EtOAc:Hex), R_{f}=0.4; ¹H NMR (400 MHz, CDCl₃) δ 8.20 (d, 1H, J=1.6 Hz), 8.09 (dd, 1H, J=8.8 Hz, J=2.0 Hz), 7.35 (d, 2H, J=8.4 Hz), 7.09 (d, 1H, J=8.4 Hz), 6.93 (d, 2H, J=8.4 Hz), 5.21 (s, 2H), 3.82 (s, 3H), 2.58 (s, 3H).

### 2-Azido-1-(4-(4-methoxybenzyloxy)-3-(trifluoromethyl)phenyl)ethanone

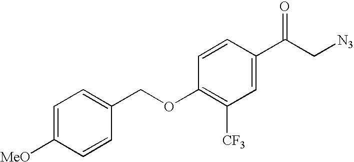

The title product was obtained according to general procedure (Scheme 1). The product was purified by silica gel column chromatography using the Combi-Flash system (Hex:EtOAc) as yellow solid in 76% (3.64 g) yield from acetophenone 2a. HPLC retention time on a C8(2) column (30×3.00 mm, 3μ) is 2.20 min with gradient 50-98% acetonitrile-H₂O (0.1% TFA) in 4.0 min as mobile phase. TLC (1:3 EtOAc:Hex), R_{f}=0.3; ¹H NMR (400 MHz, CDCl₃) δ 8.15 (d, 1H, J=2.0 Hz), 8.05 (dd, 1H, J=8.8 Hz, J=2.4 Hz), 7.33-7.36 (m, 2H), 7.12 (d, 1H, J=8.8 Hz), 6.90-6.94 (m, 2H), 5.22 (s, 2H), 4.51 (s, 2H), 3.82 (s, 3H).

### (R)-tert-Butyl 4-(2-(4-(4-methoxybenzyloxy)-3-(trifluoromethyl)phenyl)-2-oxoethylcarbamoyl)-2,2,4-trimethyloxazolidine-3-carboxylate (5a)

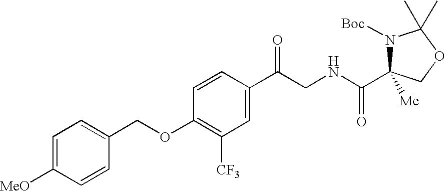

The title product was obtained according to general procedure (Scheme 1). The reaction was stirred at room temperature for 2 hours. The product was purified by silica gel column chromatography using the Combi-Flash system (Hex:EtOAc) as yellow foam in 72% (2.44 g) yield from amino-acetophenone 3a. HPLC retention time on a C8(2) column (30×3.00 mm, 3μ) is 2.51 min with gradient 50-98% acetonitrile-H₂O (0.1% TFA) in 4.0 min as mobile phase. TLC (1:1 EtOAc:Hex), R_{f}=0.5; ¹H NMR (400 MHz, CDCl₃) δ 8.24 (br s, 1H), 8.09 (br d, 1H, J=8.4 Hz), 7.32-7.39 (m, 2H), 7.12 (d, 1H, J=8.4 Hz), 6.90-6.97 (m, 2H), 5.22 (s, 2H), 4.70 (t, 2H, J=5.2 Hz), 4.30 (br s, 1H), 3.78-3.86 (m, 5H), 1.38-1.85 (m, 18H).

### (R)-tert-Butyl 4-(2-(4-hydroxy-3-(trifluoromethyl)phenyl)-2-oxoethylcarbamoyl)-2,2,4-trimethyloxazolidine-3-carboxylate (6a)

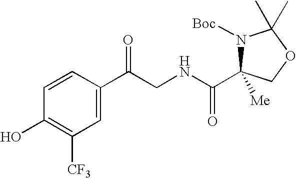

To a solution of (R)-tert-butyl 4-(2-(4-(4-methoxybenzyloxy)-3-(trifluoromethyl)phenyl)-2-oxoethylcarbamoyl)-2,2,4-trimethyloxazolidine-3-carboxylate (4) (2.4 g, 4.1 mmol, 1.0 equiv) in methanol (20 mL) was added 10% Pd/C (240 mg). The reaction mixture was stirred for 3 hours at rt under H₂ atmosphere using a H₂ balloon, filtered through celite and concentrated to give (R)-tert-butyl 4-(2-(4-hydroxy-3-(trifluoromethyl)phenyl)-2-oxoethylcarbamoyl)-2,2,4-trimethyloxazolidine-3-carboxylate (6a) as a white foam in quantitative yield. HPLC retention time on a C18 column (30×4.6 mm, 3.5 pt) was 2.62 min with gradient 10-95% acetonitrile-H₂O (0.1% TFA) in 3.5 min as mobile phase. MS (ESI, M+H⁺)=461.4.

### (R)-tert-Butyl 2,2,4-trimethyl-4-(2-oxo-2-(4-(2-phenethoxyethoxy)-3-(trifluoromethyl)phenyl)ethylcarbamoyl)oxazolidine-3-carboxylate

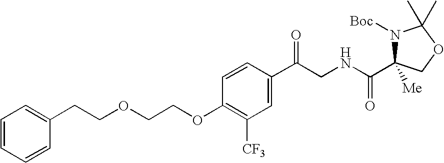

To a solution of (R)-tert-butyl 4-(2-(4-hydroxy-3-(trifluoromethyl)phenyl)-2-oxoethylcarbamoyl)-2,2,4-trimethyloxazolidine-3-carboxylate (114 mg, 0.25 mmol, 1.0 equiv) and 2-phenethoxyethanol (42 mg, 0.25 mmol, 1.0 equiv) in DCM (1 mL) was added polymer bond PPh₃ (125 mg, 0.75 mmol, 3.0 equiv). The reaction mixture was stirred at rt for 0.5 hour and cooled to 0° C. A solution of DIAD (0.053 mL, 0.25 mmol, 1.0 equiv) in DCM (0.5 mL) was added drop wise to the reaction mixture. The reaction mixture was stirred at rt for 2 hours, filtered and evaporated under reduced pressure to give a residue, which was purified by SiO₂ column chromatograph (30-50% EtOAc in hexanes) to give (R)-tert-butyl 2,2,4-trimethyl-4-(2-oxo-2-(4-(2-phenethoxyethoxy)-3-(trifluoromethyl)phenyl)ethylcarbamoyl)oxazolidine-3-carboxylate in 32% (48 mg) yield. HPLC retention time on a C18 column (30×4.6 mm, 3.5μ) was 2.34 min with gradient 50-95% acetonitrile-H₂O (0.1% TFA) in 3.5 min as mobile phase. MS (ESI, M+H⁺)=609.5.

### (R)-tert-Butyl 2,2,4-trimethyl-4-(2-oxo-2-(4-(4-phenoxybutoxy)-3-(trifluoromethyl)phenyl)ethylcarbamoyl)oxazolidine-3-carboxylate

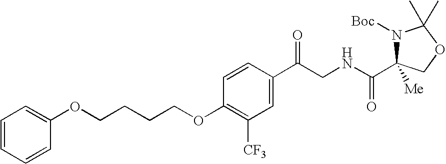

The title compound was prepared from (R)-tert-butyl 4-(2-(4-hydroxy-3-(trifluoromethyl)phenyl)-2-oxoethylcarbamoyl)-2,2,4-trimethyloxazolidine-3-carboxylate (0.25 mmol, 1.0 equiv) and 4-phenoxybutan-1-ol (0.25 mmol, 1.0 equiv) according to the general procedure in 39% yield. HPLC retention time on a C18 column (30×4.6 mm, 3.5μ) was 2.62 min with gradient 50-95% acetonitrile-H₂O (0.1% TFA) in 3.5 min as mobile phase. MS (ESI, M+H⁺)=609.5.

### (R)-tert-Butyl 2,2,4-trimethyl-4-(5-(4-(2-phenethoxyethoxy)-3-(trifluoromethyl)phenyl)thiazol-2-yl)oxazolidine-3-carboxylate (7a)

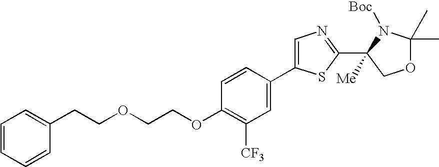

To a solution of (R)-tert-butyl 2,2,4-trimethyl-4-(2-oxo-2-(4-(2-phenethoxyethoxy)-3-(trifluoromethyl)phenyl)ethylcarbamoyl)oxazolidine-3-carboxylate (48 mg, 0.079 mmol, 1.0 equiv) in toluene (1 mL) was added Lawesson's reagent (32 mg, 0.087 mmol, 1.1 equiv). The reaction mixture was heated at 80° C. for 3 h. The crude product was purified directly by SiO₂ column chromatograph (EtOAc/hexanes, 3:7) to give (R)-tert-butyl 2,2,4-trimethyl-4-(5-(4-(2-phenethoxyethoxy)-3-(trifluoromethyl)phenyl)thiazol-2-yl)oxazolidine-3-carboxylate. HPLC retention time on a C18 column (30×4.6 mm, 3.5μ) was 3.23 min with gradient 50-95% acetonitrile-H₂O (0.1% TFA) in 3.5 min as mobile phase. MS (ESI, M+H⁺)=607.5.

### (R)-tert-Butyl 2,2,4-trimethyl-4-(5-(4-(4-phenoxybutoxy)-3-(trifluoromethyl)phenyl)thiazol-2-yl)oxazolidine-3-carboxylate (7b)

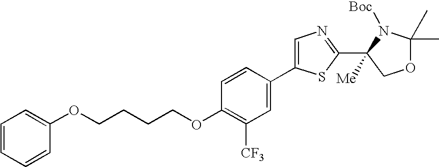

The title compound was prepared from (R)-tert-butyl 2,2,4-trimethyl-4-(2-oxo-2-(4-(4-phenoxybutoxy)-3-(trifluoromethyl)phenyl)ethylcarbamoyl)oxazolidine-3-carboxylate. HPLC retention time on a C8(2) column (30×50 mm, 3μ) is 2.66 min with gradient 70-98% acetonitrile-H₂O (0.1% TFA) in 3.5 min as mobile phase. MS (ESI, M+H⁺)=607.0.

### (S)-2-Amino-2-(5-(4-(2-phenethoxyethoxy)-3-(trifluoromethyl)phenyl)thiazol-2-yl)propan-1-ol (8a)

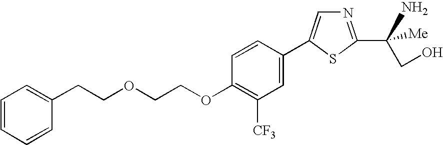

The title compound was prepared from (R)-tert-butyl 2,2,4-trimethyl-4-(5-(4-(2-phenethoxyethoxy)-3-(trifluoromethyl)phenyl)thiazol-2-yl)oxazolidine-3-carboxylate. HPLC retention time on a C18 column (30×4.6 mm, 3.5μ) was 1.98 min with gradient 10-95% acetonitrile-H₂O (0.1% TFA) in 3.5 min as mobile phase. MS (ESI, M+H⁺)=467.3; ¹H NMR (400 MHz, CDCl₃) δ 7.77 (s, 1H), 7.66 (d, 1H, J=2 Hz), 7.54 (dd, 1H, J=8.4 Hz, J=2.0 Hz), 7.29-7.17 (m, 5H), 7.00 (d, 1H, J=8.4 Hz), 4.20 (t, 2H, J=4.8 Hz), 4.04 (br s, 2H), 3.84 (t, 2H, J=4.8 Hz), 3.78 (t, 2H, J=7.0 Hz), 2.90 (t, 2H, J=7.0 Hz), 1.83 (s, 3H).

### (S)-2-Amino-2-(5-(4-(4-phenoxybutoxy)-3-(trifluoromethyl)phenyl)thiazol-2-yl)propan-1-ol (8b)

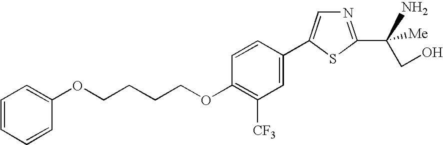

The title compound was prepared from (R)-tert-butyl 2,2,4-trimethyl-4-(5-(4-(4-phenoxybutoxy)-3-(trifluoromethyl)phenyl)thiazol-2-yl)oxazolidine-3-carboxylate. HPLC retention time on a C18 column (30×4.6 mm, 3.5μ) was 2.18 min with gradient 10-95% acetonitrile-H₂O (0.1% TFA) in 3.5 min as mobile phase. MS (ESI, M+H⁺)=467.4; ¹H NMR (400 MHz, CDCl₃) δ 7.77 (s, 1H), 7.66 (d, 1H, J=2.4 Hz), 7.57 (dd, 1H, J=8.6 Hz, J=2.0 Hz), 7.29-7.25 (m, 2H), 7.00 (d, 1H, J=8.4 Hz), 6.95-6.88 (m, 3H), 4.14 (t, 2H, J=5.6 Hz), 4.05-4.02 (m, 4H), 2.06-1.98 (m, 4H), 1.82 (s, 3H).
General Method for Phosphate Synthesis 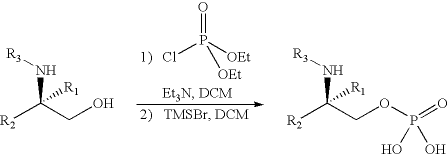
R₃═H or protecting group

Synthetic strategy for synthesis of desired phosphates is illustrated above. To a solution of unprotected amino alcohol (1.0 equiv) in dry CH₂Cl₂ at RT was added excess diethyl chlorophosphate (10.0 equiv) and triethylamine (20.0 equiv) and the reaction stirred for 12-18 hours. The reaction was monitored by LC-MS. The crude reaction mixture was then evaporated to dryness in vacuo. The obtained phospho-diester intermediate was reacted with excess bromotrimethylsilane (10.0-20.0 equiv) in dry CH₂Cl₂ at RT over a period of 6-10 hours to afford the final phosphate which was purified by reverse-phase preparative HPLC after evaporation of the solvent and excess reagent.

### (S)-2-Amino-2-(5-(4-(4-phenoxybutoxy)-3-(trifluoromethyl)phenyl)-1,3,4-thiadiazol-2-yl)propyl dihydrogen phosphate (a)

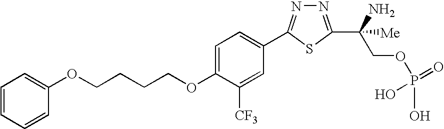

The title product was obtained as a white solid in 11% (2 mg) yield from the alcohol precursor. HPLC retention time on a C18 column (30×4.6 mm, 3.5μ) was 2.06 min with gradient 10-95% acetonitrile-H₂O (0.1% TFA) in 3.5 min as mobile phase. MS (ESI, M+H⁺)=548.3. NMR (400 MHz, CD₃OD) δ 8.23-8.17 (m, 2H), 7.37 (d, 1H, J=8.4 Hz), 7.25-7.21 (m, 2H), 6.98-6.87 (m, 3H), 4.30-4.24 (m, 4H), 4.06 (t, 2H, J=6.0 Hz), 2.08-1.88 (m, 4H), 1.88 (s, 3H).

### (S)-2-Amino-2-(5-(4-(3-phenoxypropoxy)-3-(trifluoromethyl)phenyl)-1,3,4-thiadiazol-2-yl)propyl dihydrogen phosphate (b)

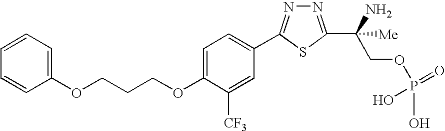

MS (ESI, M+H⁺)=533.9; HPLC retention time on a Synergi-Max RP column (2×20 mm, 2 μL) is 1.14 min with gradient 30-99% acetonitrile-H₂O (0.1% TFA) in 2 min as mobile phase.

### (S)-2-Amino-2-(5-(4-(2-phenethoxyethoxy)-3-(trifluoromethyl)phenyl)-1,3,4-thiadiazol-2-yl)propyl dihydrogen phosphate (c)

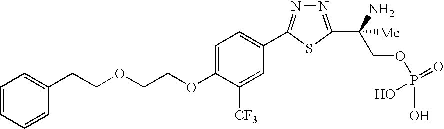

MS (ESI, M+H⁺)=547.9; HPLC retention time on a Synergi-Max RP column (2×20 mm, 2 μL) is 1.38 min with gradient 20-95% acetonitrile-H₂O (0.1% TFA) in 2 min as mobile phase.

### (S)-2-Amino-2-(5-(4-(4-(4-fluorophenoxy)butoxy)-3-(trifluoromethyl)phenyl)-1,3,4-thiadiazol-2-yl)propyl dihydrogen phosphate (d)

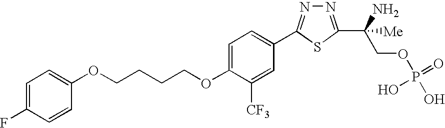

HPLC retention time on a Synergi MAX-RP 100A (20×2 mm, 2 g) was 1.55 min with gradient 20-95% acetonitrile-H₂O (0.1% TFA) in 2.0 min as mobile phase. MS (ESI, M+H⁺)=565.9

### (S)-2-Amino-2-(5-(4-(4-(2-fluorophenoxy)butoxy)-3-(trifluoromethyl)phenyl)-1,3,4-thiadiazol-2-yl)propyl dihydrogen phosphate (e)

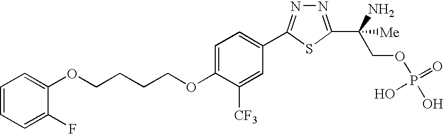

HPLC retention time on a Synergi MAX-RP 100A (20×2 mm, 2μ) was 1.55 min with gradient 20-95% acetonitrile-H₂O (0.1% TFA) in 2.0 min as mobile phase. MS (ESI, M+H⁺)=565.9

### (S)-2-Amino-2-(5-(4-(4-(3-fluorophenoxy)butoxy)-3-(trifluoromethyl)phenyl)-1,3,4-thiadiazol-2-yl)propyl dihydrogen phosphate (f)

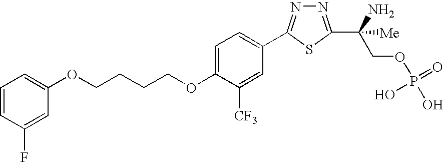

HPLC retention time on a Synergi MAX-RP 100A (20×2 mm, 2 g) was 1.55 min with gradient 20-95% acetonitrile-H₂O (0.1% TFA) in 2.0 min as mobile phase. MS (ESI, M+H⁺)=566.0

### (S)-2-Amino-2-(5-(4-(2-(pentyloxy)ethoxy)-3-(trifluoromethyl)phenyl)thiazol-2-yl)propyl dihydrogen phosphate (g)

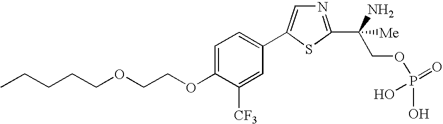

The title product was obtained according to general procedure (Scheme 2) from compound 7a. MS (ESI, M+H⁺)=513.0

### (S)-2-Amino-2-(5-(4-(2-phenethoxyethoxy)-3-(trifluoromethyl)phenyl)thiazol-2-yl)propyl dihydrogen phosphate (h)

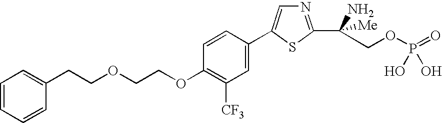

The title compound was prepared from (S)-2-amino-2-(5-(4-(2-phenethoxyethoxy)-3-(trifluoromethyl)phenyl)thiazol-2-yl)propan-1-ol. HPLC retention time on a C18 column (30×4.6 mm, 3.5μ) was 2.03 min with gradient 10-95% acetonitrile-H₂O (0.1% TFA) in 3.5 min as mobile phase. MS (ESI, M+H⁺)=547.4

### (S)-2-Amino-2-(5-(4-(4-phenoxybutoxy)-3-(trifluoromethyl)phenyl)thiazol-2-yl)propyl dihydrogen phosphate (i)

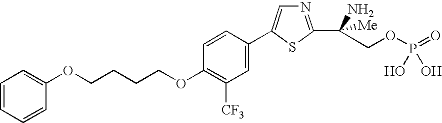

The title compound was prepared from (S)-2-amino-2-(5-(4-(4-phenoxybutoxy)-3-(trifluoromethyl)phenyl)thiazol-2-yl)propan-1-ol. HPLC retention time on a C18 column (30×4.6 mm, 3.5,) was 2.17 min with gradient 10-95% acetonitrile-H₂O (0.1% TFA) in 3.5 min as mobile phase. MS (ESI, M+H⁺)=547.5.

### (S)-2-Amino-2-(5-(4-(2-(3-phenylpropoxy)ethoxy)-3-(trifluoromethyl)phenyl)-1,3,4-thiadiazol-2-yl)propyl dihydrogen phosphate (j)

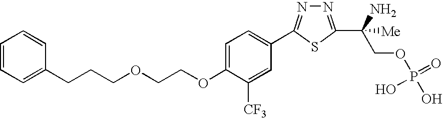

HPLC retention time on a C8(2) column (30×50 mm, 3 μL) is 1.85 min with gradient 30-98% acetonitrile-H₂O (0.1% TFA) in 3.5 min as mobile phase. MS (ESI, M+H⁺)=561.99

### (S)-2-Amino-2-(5-(4-(4-propoxybutoxy)-3-(trifluoromethyl)phenyl)-1,3,4-thiadiazol-2-yl)propyl dihydrogen phosphate (k)

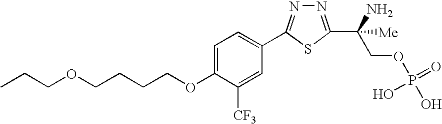

HPLC retention time on a C8(2) column (30×50 mm, 3 μL) is 1.67 min with gradient 30-98% acetonitrile-H₂O (0.1% TFA) in 3.5 min as mobile phase. MS (ESI, M+H⁺)=514.0

### (S)-2-Amino-2-(5-(4-(4-(benzyloxy)butoxy)-3-(trifluoromethyl)phenyl)-1,3,4-thiadiazol-2-yl)propyl dihydrogen phosphate (l)

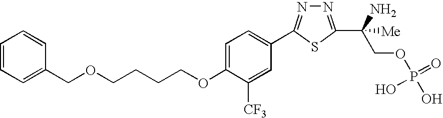

HPLC retention time on a C8(2) column (30×50 mm, 3 μL) is 1.84 min with gradient 30-98% acetonitrile-H₂O (0.1% TFA) in 3.5 min as mobile phase. MS (ESI, M+H⁺)=562.0

### Examples of specific methods used to make (S)-2-Amino-2-(5-(4-(2-(pentyloxy)ethoxy)-3-(trifluoromethyl)phenyl)-1,3,4-thiadiazol-2-yl)propan-1-ol

Description 1

### 4-(2-(Pentyloxy)ethoxy)-3-(trifluoromethyl)benzoic acid (D1)

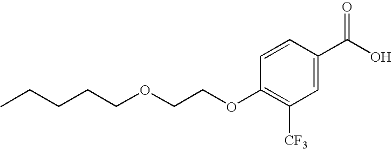

2-(pentyloxy)ethanol (318 mg, 2.4 mmol, 1 equiv) was stirred with potassium t-butyloxide (6 mL, 1M solution in THF, 6.0 mmol, 2.5 equiv), THF (10 mL) at 75° C. for 10 minutes. 4-Fluoro-3-trifluoromethylbenzoic acid was added and the mixture heated at 75° C. overnight. The mixture was then condensed, diluted in water, acidified, extracted with ethyl acetate and dried over Na₂SO₄. The organic layers were condensed to provide the title product.

Description 1 Alternative Method (D1A)

### 4-(2-(Pentyloxy)ethoxy)-3-(trifluoromethyl)benzoic acid (D1)

A 5 L round bottom flask was inserted and charged with a solution of n-pentyloxyethanol (82.5 g, 0.62 mol, 1.1 equiv), THF (1.2 L), and 1.0 M potassium t-butoxide in THF (1417 mL, 1.4 mol, 2.5 equiv) at room temperature (18 to 23° C.). The mixture was heated to 65° C. After 15 minutes, a solution of 4-fluoro-3 (trifluoromethyl)benzoic acid (118 g, 0.56 mol) in THF (1256 mL) was charged slowly over 30 minutes. No frothing was observed. After 2.5 hours, the reaction was found to be complete by HPLC. The reaction mixture was cooled to ambient temperature (18 to 23° C.) and stirred overnight. The reaction mixture was quenched with water (1400 mL), concentrated to remove THF, then adjusted to a pH of 2 with 6 N HCl. The mixture was extracted twice with MTBE (750 mL, 150 mL), the organics were combined, dried with magnesium sulfate, filtered, and concentrated under vacuum to afford the title product (188 g, 104% yield, 92.8% AUC by HPLC).

Description 2

### 4-(2-(Pentyloxy)ethoxy)-3-(trifluoromethyl)benzohydrazide (D2)

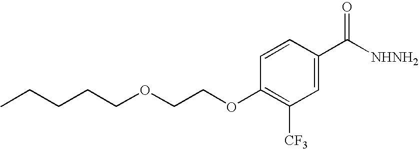

4-(2-(Pentyloxy)ethoxy)-3-(trifluoromethyl)benzoic acid (D1) (2.4 mmol, 1 equiv) was stirred with HATU (1.094 g, 2.88 mmol, 1.2 equiv) and DIEA (2.086 mL, 12 mmol, 5.0 equiv) in a mixture of DCM-DMF (2:1, 15 mL) and hydrazine (226 μL, 7.2 mmol. 3.0 equiv). The mixture was then diluted with ethyl acetate and washed with water and brine. The organic layer was dried over Na₂SO₄ and condensed to give the title product.

Description 2 Alternative Method (D2A)

### 4-(2-(Pentyloxy)ethoxy)-3-(trifluoromethyl)benzohydrazide (D2)

A solution of CDI (138 g, 1.5 equiv), in THF (500 mL) was treated with a solution of 4-(2-(pentyloxy)ethoxy)-3-(trifluoromethyl)benzoic acid (DIA) (181.61 g, 0.57 mol) in THF (1420 mL) over 15 minutes. The resultant solution was then stirred for one hour at room temperature before adding into a solution of hydrazine (142 g, 5 equiv) in THF (1000 mL) over 30 min. The reaction was monitored by TLC and deemed complete after 1 h. The reaction mixture was cooled to ambient temperature, treated with brine (1000 mL), extracted with MTBE (1×1000 mL, 1×250 mL), dried, and concentrated to afford the title product (278 g, 147% yield, 82.0% AUC by HPLC) as a foamy solid.

Description 3

### (R)-3-(tert-Butoxycarbonyl)-2,2,4-trimethyloxazolidine-4-carboxylic acid (D3)

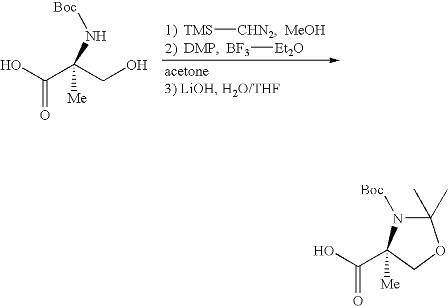

To a solution of the (S)-2-(tert-butoxycarbonylamino)-3-hydroxy-2-methylpropanoic acid (5.0 g, 1.0 equiv) in CH₂Cl₂/MeOH (4:1, 50 mL) was added a solution of TMS-CHN₂.

The residue was dissolved in acetone (30 mL) and 2,2-dimethoxypropane (DMP) (12 mL). To the mixture was added BF₃—OEt₂ (2 mL) drop-wise and the solution was stirred at RT for 4 hours. The solvent was removed in vacuo and the product was purified by silica gel column chromatography using the Isco system (0-30% Hex:EtOAc) to give the oxazolidine methyl ester intermediate.

The purified residue was dissolved in H₂O:THF (1:4) and to the solution was added LiOH (1.16 g). The solution was heated at refluxed overnight, cooled to room temperature and condensed to remove the THF. The aqueous material was diluted with H₂O (100 mL), acidified to pH˜2 with 10% KHSO₄ and then extracted with EtOAc. The organic layer was dried over Na₂SO₄, and condensed to afford the title compound in 66% yield (3.29 g).

Description 3 Alternative Method (D3A)

### (R)-3-(tert-Butoxycarbonyl)-2,2,4-trimethyloxazolidine-4-carboxylic acid (D3)

A 22 L round bottom flask was inserted and charged with N-Boc-α-methyl-L-serine 4 (564 g, 2.57 mol), acetone (8.4 L) and stirred. The mixture was slowly charged with 1,8-Diazabicyclo[5.4.0]undec-7-ene (770 mL, 5.1 mol, 2 equiv). The addition was exothermic and the temperature was maintained below 25° C. The mixture was stirred for 45 minutes at ambient conditions, and then cautiously charged with iodomethane (320 mL, 5.1 mol, 2 equiv). The addition was exothermic and the temperature was maintained below 25° C. The mixture was allowed to stir overnight at room temperature (18 to 23° C.). After 16 hours, TLC indicated starting material remained. The reaction was charged with iodomethane (320 mL, 5.1 mol, 2 equiv), warmed to 30° C. for 4 hours, and then allowed to stir overnight at room temperature (18 to 23° C.). After 16 hours, assay by TLC indicated the reaction was complete. The reaction mixture was combined with another reaction mixture of a scale of 275 g. The combined reaction mixtures were concentrated under vacuum to a residue, transferred into a reactor, charged with water (8.4 L), ethyl acetate (8.4 L), mixed thoroughly, phases split, extracted aqueous phase once more with ethyl acetate (8.4 L), combined organic phases, washed with 5% w/v citric acid (900 mL), brine (1 L), dried with magnesium sulfate, filtered over Celite, and concentrated to afford the product 5 (925 g, 104% yield) as an oil. The material was used as is for the next step.

A 22 L round bottom flask was charged with crude compound N-Boc-x-methyl-L-serine methyl ester (925 g, 3.8 mol based on theoretical output from previous step), dichloromethane (10 L), 2,2-Dimethoxypropane (2.6 L), and mixed. Boron trifluoride diethyl etherate (200 mL, 1.62 mol, 0.42 equiv) in dichloromethane (1.2 L) was cautiously charged over 45 minutes. The resulting dark solution was stirred over night at room temperature (18 to 23° C.). After 16 hours TLC indicated the reaction was complete. The mixture was slowly quenched with saturated sodium bicarbonate (3.5 L) while maintaining the temperature below 25° C. Once the quench was complete, the mixture was stirred for 30 minutes, the phases separated, and the aqueous extracted with dichloromethane (3.5 L), the organic phases were combined, washed with saturated sodium bicarbonate (3 L), concentrated to obtain corresponding oxazolidine methyl ester (1070 g, quantitative) as a yellow color oil. The material was used as is for the next step.

A 22 L round bottom flask was charge with lithium hydroxide monohydrate (482 g, 11.4 mol, 3 equiv), water (2.3 L), methanol (2.1 L), a solution of crude corresponding oxazolidine methyl ester (1046 g—based on theoretical output of previous step, 3.82 mol) in tetrahydrofuran (6.5 L). The mixture was stirred for 72 hours at room temperature (18 to 23° C.). TLC indicated the reaction was complete. The mixture was concentrated under vacuum at 40° C., the residue was charged with water (10 L), MTBE (6 L), mixed thoroughly, and the phases split. The organic phase was washed with water (4 L), the aqueous phases were combined, and solid citric acid was charged in portions until a pH of 3 was obtained. The aqueous was extracted with ethyl acetate (2×10 L), ethyl acetate phases were combined, washed with brine (7 L), dried with magnesium sulfate, filtered over Celite, and concentrated to afford the title product (770 g, 77.6% yield) as an off white solid.

Description 4

### (R)-tert-butyl 2,2,4-trimethyl-4-(5-(4-(2-(pentyloxy)ethoxy)-3-(trifluoromethyl)-phenyl)-1,3,4-thiadiazol-2-yl)oxazolidine-3-carboxylate (D4)

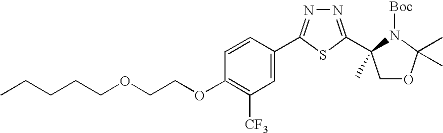

To a solution of (R)-3-(tert-butoxycarbonyl)-2,2,4-trimethyloxazolidine-4-carboxylic acid (D3) (100 mg, 0.386 mmol, 1 equiv), HATU (176 mg, 0.463 mmol,), and DIEA (0.335 mL,) in DCM:DMF (2:1, 3 mL) was added 4-(2-(pentyloxy)ethoxy)-3-(trifluoromethyl)benzohydrazide (D2) (281 mg, 0.463 mmol, 1.2 equiv). The products were purified on a column (resulting in 235 mg of material). MS (ESI): 575.82 (MH⁺); ¹H NMR (400 MHz, CDCl₃) δ 9.96 (br s, 1H), 8.92 (br, 1H), 8.05 (d, 1H, J=2.4 Hz), 7.95 (dd, 1H, J=8.8 Hz, J=2.4 Hz), 7.06 (d, 1H, J=8.8 Hz), 4.55 (br s, 1H), 4.25 (t, 2H, J=4.4 Hz), 3.83 (t, 2H, J=5.2 Hz), 3.78 (br, 1H), 3.54 (t, 2H, J=6.8 Hz), 1.68 (s, 6H), 1.58 (m, 3H), 1.52 (s, 9H), 1.33-1.29 (m, 4H), 0.89 (t, 3H, J=5.6 Hz).

The obtained material (235 mg) was dissolved in toluene (15 mL) with Lawesson's reagent (468 mg, 1.16 mmol, 3 equiv). The resultant mixture was heated at 85° C. for 1-3 hours to produce the title product which was then purified using two silica columns (0-30% hexane:ethyl acetate (2:1), DCM, DCM/MeOH) (180 mg). MS (ESI): 574.16 (MH⁺), HPLC retention time on a C8(2) column (30×3.00 mm, 3μ) is 3.42 min with gradient 50-98% acetonitrile-H₂O (0.1% TFA) in 3.5 min as mobile phase.

Description 4 Alternative Method (D4A)

### (R)-tert-Butyl 2,2,4-trimethyl-4-(5-(4-(2-(pentyloxy)ethoxy)-3-(trifluoromethyl)-phenyl)-1,3,4-thiadiazol-2-yl)oxazolidine-3-carboxylate (D4)

A slurry of HATU (314 g, 1.2 equiv), in 600 mL of dimethylformamide (DMF) was treated with a solution oxazolidine acid 7 (178 g, 0.69 mol) in 1400 mL of dichloromethane (CH₂Cl₂) and DIPEA (244 mL, 2.03 equiv) over 15 minutes. The resultant solution was then stirred for one hour at room temperature. A solution of 4-(2-(pentyloxy)ethoxy)-3-(trifluoromethyl)benzohydrazide (D2A) (230 g, 0.69 mol) in CH₂Cl₂ (1800 mL) and DMF (700 mL) was charged slowly to the aforementioned mixture over 15 minutes. The reaction was monitored by TLC and deemed complete after 45 minutes. The reaction mixture was concentrated to remove CH₂Cl₂. The mixture was charged with water (2.5 L) and ethyl acetate (2.5 L), the phases were split, the organic phase was washed twice with water (2×1 L), brine (2×1 L), dried with magnesium sulfate, filtered, and concentrated to afford the crude product (489 g, 123% yield). The crude oil was then purified by column chromatography using silica-gel (1.5 kg), eluted with 5% ethyl acetate: 95% heptane to 25% ethyl acetate: 75% heptane to afford the product (450 g, 113% yield, 92.7% AUC by HPLC) as a yellow oil.

A 12 L round bottom flask was inserted and charged with Lawesson's reagent (334 g, 0.82 mol, 1.2 equiv), a solution of the yellow oil (396 g, 0.68 mol) in toluene (3 L), and the mixture heated to 80° C. and held for 2 hours, at which point it was assayed by HPLC and found to be complete. The mixture was cooled to 30° C. and charged with ethyl acetate (1.5 L), saturated aqueous sodium bicarbonate (1.5 L), and brine (1.5 L). The mixture was mixed thoroughly, the phases were separated, and the aqueous extracted once more with ethyl acetate (1 L). The organic phases were combined, washed with brine (1 L), saturated with sodium bicarbonate (1 L), dried with magnesium sulfate, filtered, and concentrated to a residue. The residue was purified by column chromatography using silica-gel (1.5 kg), eluted with 5% ethyl acetate: 95% heptane to afford the title compound (340 g, 86% yield, 82.7% AUC by HPLC) as a brown oil.

### Example 1

### (S)-2-Amino-2-(5-(4-(2-(pentyloxy)ethoxy)-3-(trifluoromethyl)phenyl)-1,3,4-thiadiazol-2-yl)propan-1-ol (E1)

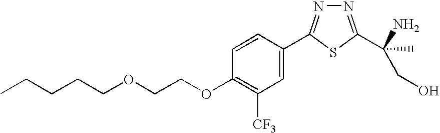

A solution of (R)-tert-butyl 2,2,4-trimethyl-4-(5-(4-(2-(pentyloxy)ethoxy)-3-(trifluoromethyl)-phenyl)-1,3,4-thiadiazol-2-yl)oxazolidine-3-carboxylate (1.0 equiv) in 20% TFA/DCM was stirred at room temperature for 1 hr. Half of the reaction mixture was purified by preparative HPLC (31 mg). MS (ESI): 434.03 (MH⁺); ¹H NMR (400 MHz, DMSO-d₆) δ 8.63 (br s, 2H), 8.23 (dd, 1H, J=8.8 Hz, J=2.4 Hz), 8.16 (d, 1H, J=2.4 Hz), 7.49 (d, 1H, J=8.8 Hz), 4.36 (t, 2H, J=4.4 Hz), 3.83 (d, 1H, J=11.2 Hz), 3.76 (d, 1H, J=11.2 Hz), 3.74 (t, 2H, J=5.2 Hz), 3.46 (t, 2H, J=6.8 Hz), 1.69 (s, 3H), 1.48 (m, 2H), 1.25 (m, 4H), 0.83 (t, 3H, J=7.2 Hz).

### Example 1

### Alternative Method (E1A)

### (S)-2-Amino-2-(5-(4-(2-(pentyloxy)ethoxy)-3-(trifluoromethyl)phenyl)-1,3,4-thiadiazol-2-yl)propan-1-ol (E1)

A 5 L round bottom flask was charged with p-toluene sulfonic acid monohydrate (225 g, 1.18 mol, 2 equiv) and a solution of D4 (340 g, 0.59 mol) in methanol (3.2 L). The mixture was heated to 65° C. for 2 hours, then assayed by HPLC and found to be complete. The mixture was cooled to 40° C. and concentrated under vacuum to remove the methanol. The mixture was azeotroped with dioxane (1 L) to a waxy solid. The solid was slurried in dioxane (1.7 L) at 40° C., and slowly charged with 4 M HCl in dioxane (1185 mL, 4.7 mol, 8 equiv). The heavy lumpy slurry developed into a fine precipitate and was heated to 50° C. and held for 1.5 hours, during which the mixture became very thick. The stirring was increased and the mixture was allowed to cool to ambient conditions (18 to 23° C.), and stirred for 14 h. The mixture was filtered and the solids washed twice as slurry with dioxane (2×1 L). The wash was repeated twice more with MTBE (2×500 mL), the solids were then dried under vacuum at 30° C. overnight and mixed with another lot to afford the title compound as dihydrochloride salt (190 g, 63.4% yield, 98.7% AUC by HPLC) as a white solid.

### Example 2

### (S)-2-amino-2-(5-(4-(2-pentyloxy)ethoxy)-3-(trifluoromethyl)phenyl)thiazol-2-yl)propyl dihydrogen phosphate (E2)

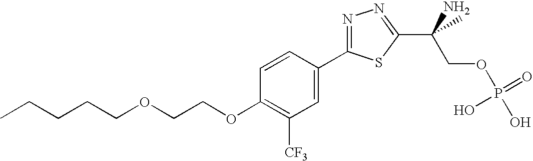

To a solution of (S)-2-amino-2-(5-(4-(2-(pentyloxy)ethoxy)-3-(trifluoromethyl)phenyl)-1,3,4-thiadiazol-2-yl)propan-1-ol (31 mg, 0.07 mmol, 1.0 equiv) in DCM was added excess diethyl chlorophosphate (101 mg, 10.0 equiv) and triethylamine (146 μL, 15.0 equiv) and the reaction. The reaction was monitored by LC-MS. The crude reaction mixture was then evaporated to dryness in vacuo, washed in NaHCO₃, condensed, diluted in ethyl acetate and dried over Na₂SO₄. The product was then condensed and separated by HPLC. The obtained phospho-diester intermediate was reacted with excess bromotrimethylsilane in dry CH₂Cl₂ (160-200 μL:2 mL) over a period of 5-6 hours. The product, a bis-TFA salt, was condensed and dissolved in [(CH₃CN:H₂O 1:1)]:DMSO, 1:3 for preparative purification. 7.7 mg of the title compounds was obtained with a purity of >95%.

MS (ESI): 514.00 (MH⁺), HPLC retention time on a C8(2) column (30×3.00 mm, 3μ) is 1.81 min with gradient 30-98% acetonitrile-H₂O (0.1% TFA) in 3.5 min as mobile phase.

1. A compound of formula I 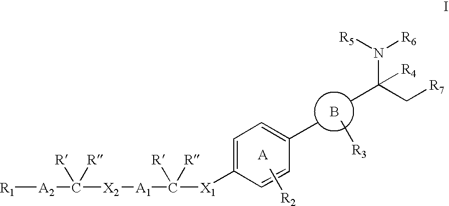
or a pharmaceutically acceptable salt thereof, wherein:
R₁ is hydrogen, halogen, cyano, alkyl, aryl, heteroaryl, cycloalkyl, heterocycloalkyl, aralkyl, heteroalykl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, haloalkyl, —CF₃, —CN, —OH, or —O-alkyl;
A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
X₁ is a bond or is CH₂, O, CH₂O, S, —S(O), —S(O)₂, —C(O)—, —C(O)O—, or NRₓ, wherein Rₓ is H or (C₁-C₆)alkyl;
X₂ is O, CH₂O, S, —S(O), —S(O)₂, —C(O)—, —C(O)O—, or NRₓ, wherein Rₓ is H or (C₁-C₆)alkyl;
R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen
R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 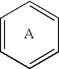
is phenyl or pyridyl; 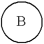
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;
R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;
R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein
Z′ is hydroxyl or halogen;
Z″ is H or halogen;
Rₚ₁ and Rₚ₂ are each independently hydrogen, C₁-C₆-alkyl, aryl, or one of the following groups: 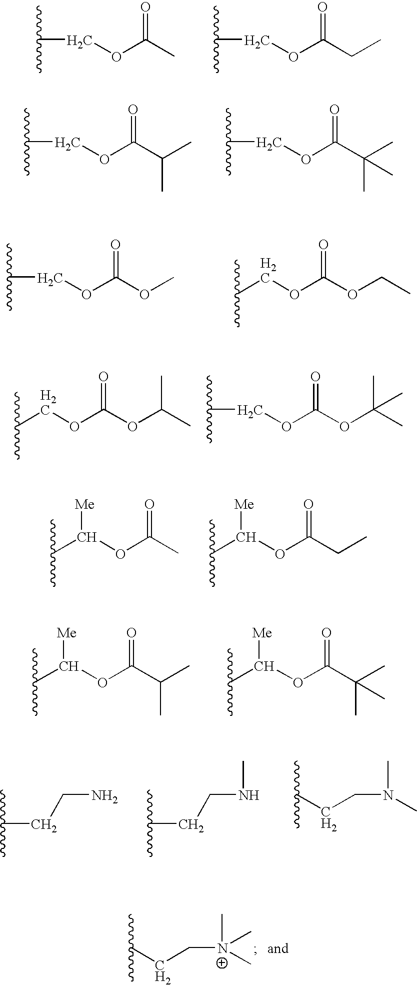
Y is heterocyclo or heteroaryl.

2. A compound of formula II 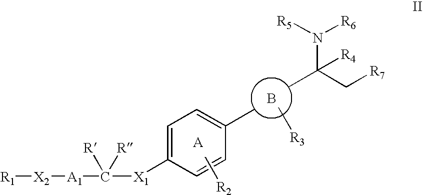
or a pharmaceutically acceptable salt thereof, wherein:
R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;
A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
X₁ is a bond or is CH₂, O, CH₂O, S, —S(O), —S(O)₂, —C(O)—, —C(O)O—, or NRC, wherein Rₓ is H or (C₁-C₆)alkyl;
X₂ is O, CH₂O, S, —S(O), —S(O)₂, —C(O)—, —C(O)O—, or NRₓ, wherein Rₓ is H or (C₁-C₆)alkyl;
R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen
R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 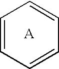
is phenyl or pyridyl; 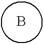
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;
R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;
R₇ is selected from the group consisting of —OH, alkylene-OH, —CO₂H, alkylene-CO₂H, -alkylene-CO₂-alkyl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂, any of which may be optionally substituted on carbon with halogen, alkyl, hydroxyl, carboxy, or alkoxy; and wherein
Z′ is hydroxyl or halogen;
Z″ is H or halogen;
Rₚ₁ and Rₚ₂ are each independently hydrogen, C₁-C₆-alkyl, aryl, or one of the following groups: 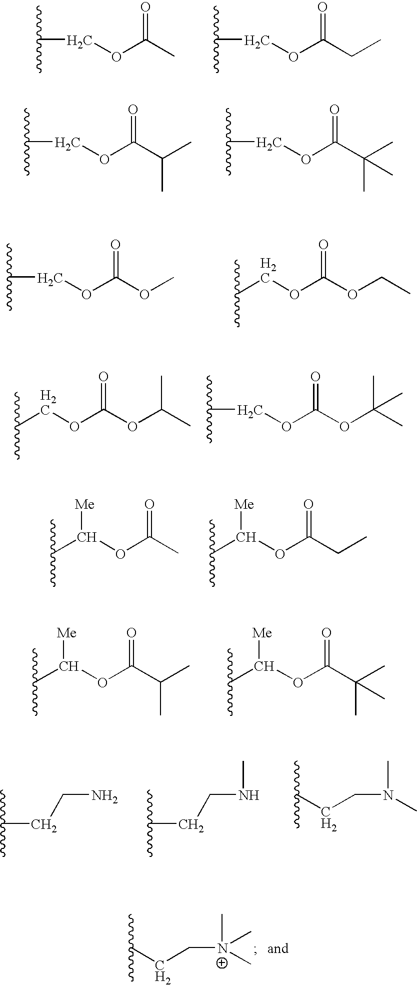
Y is heterocyclo or heteroaryl.

3. A compound which is 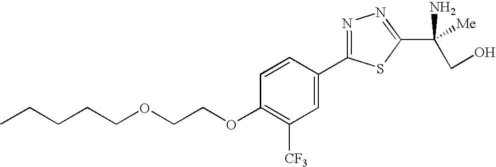 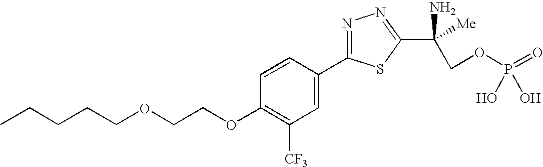 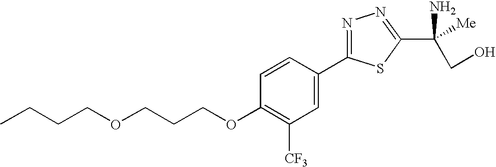 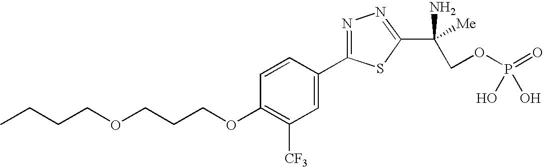 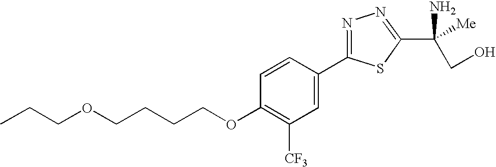 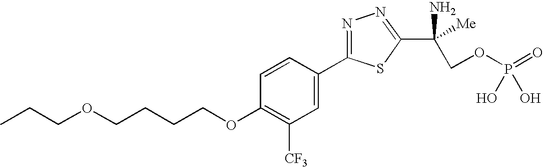 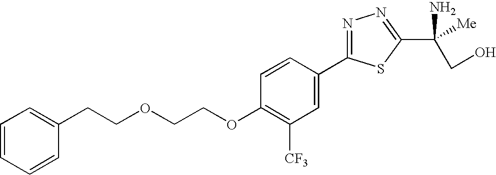 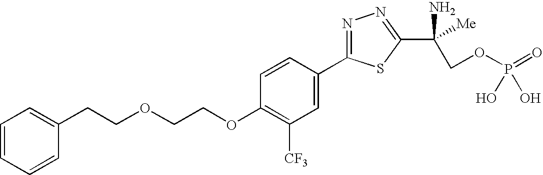 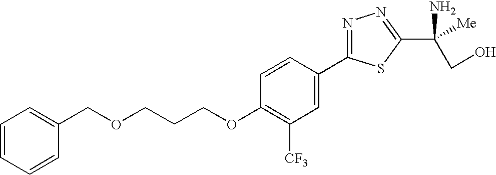 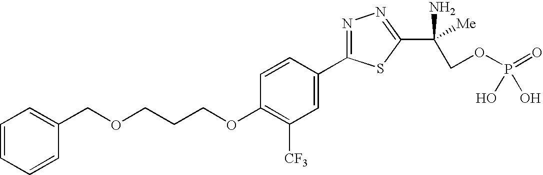 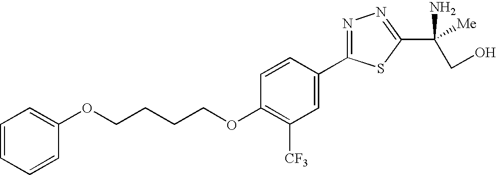 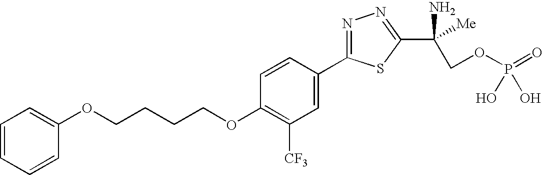 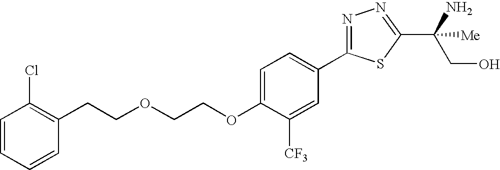 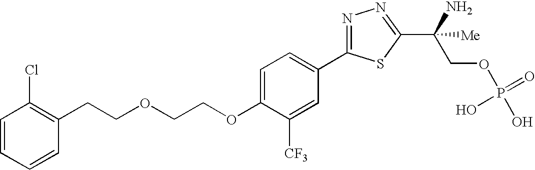 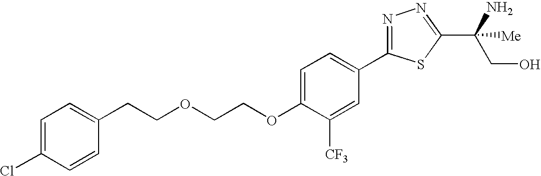 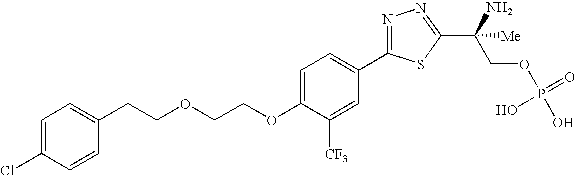 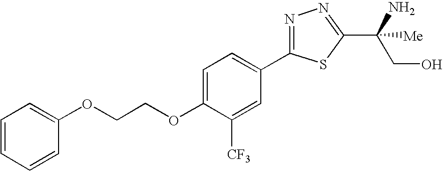 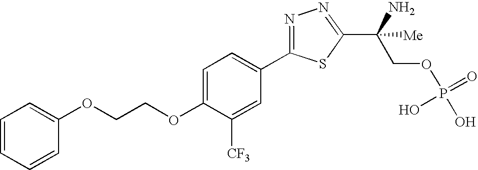 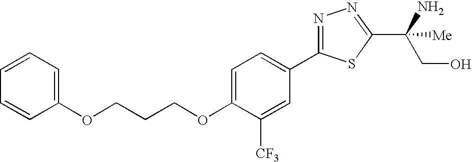 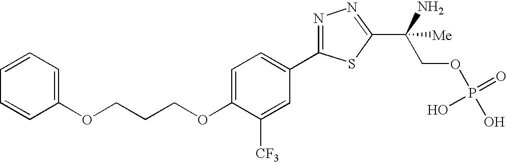 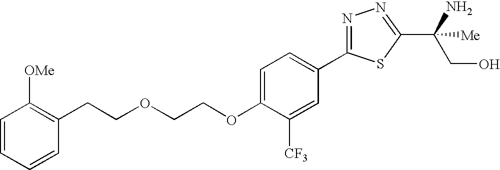 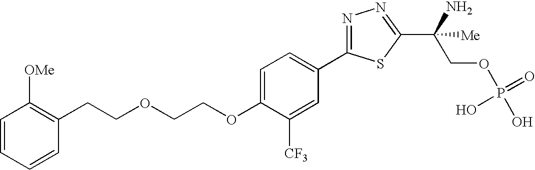 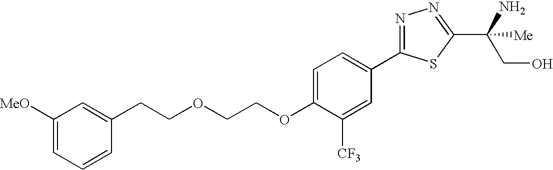 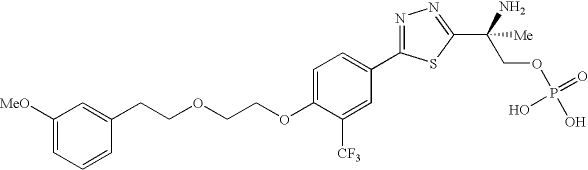 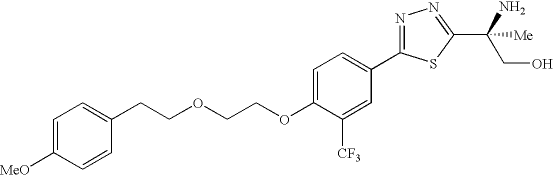 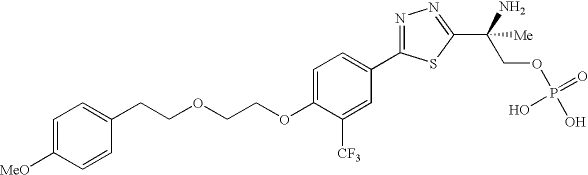 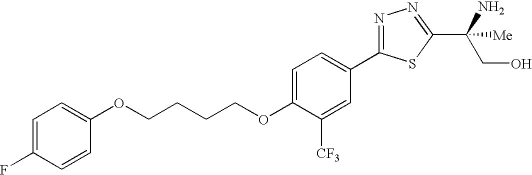 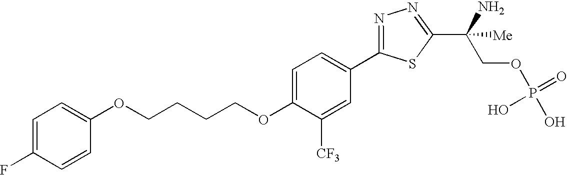 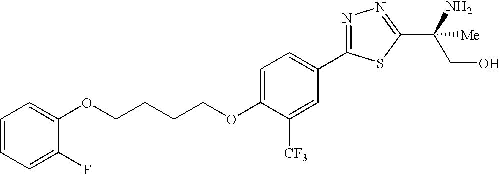 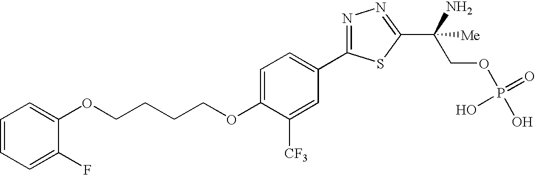 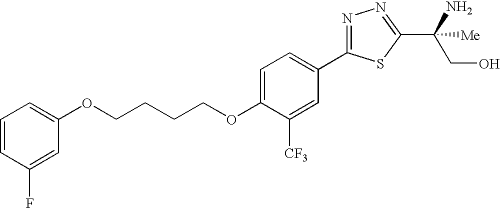 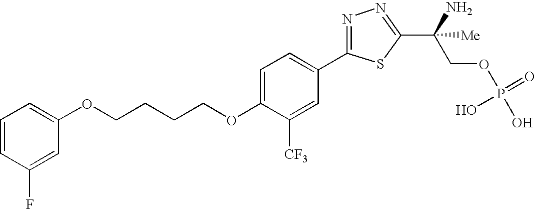 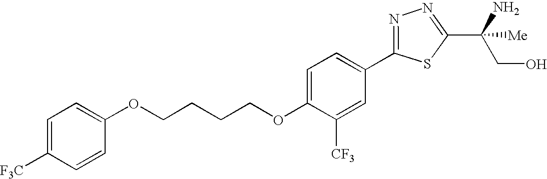 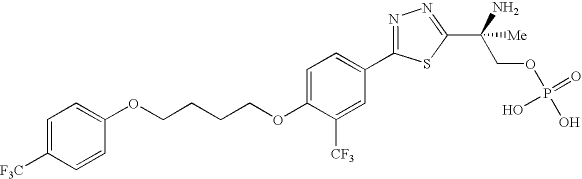 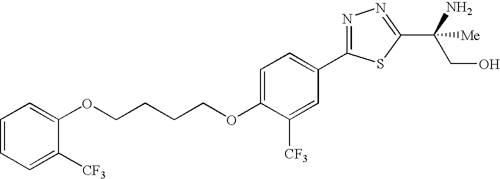 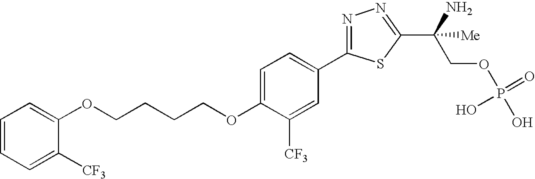 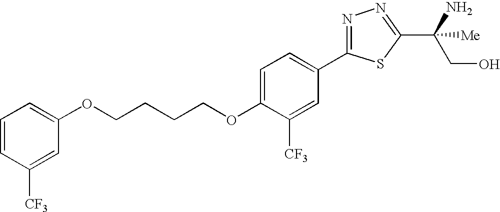 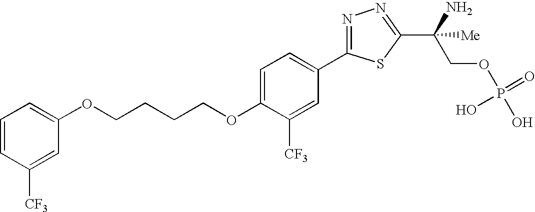 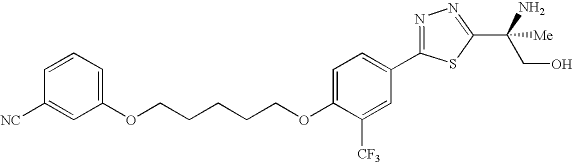 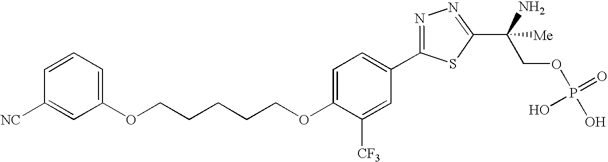 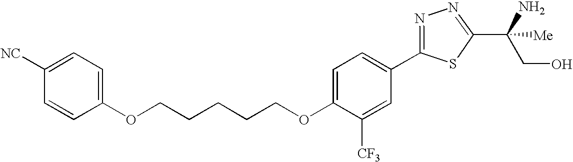 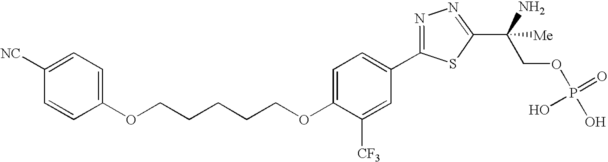 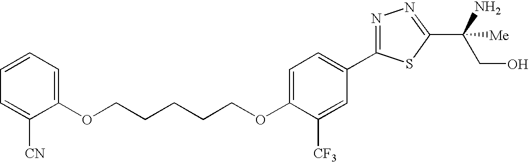 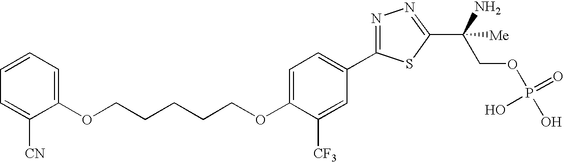 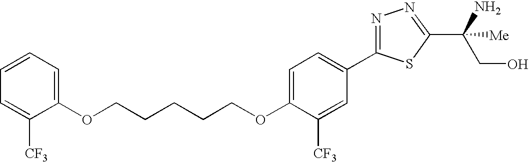 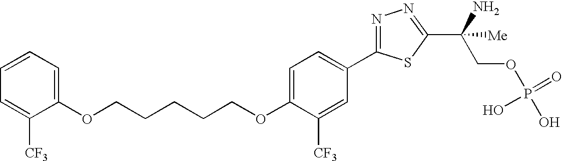 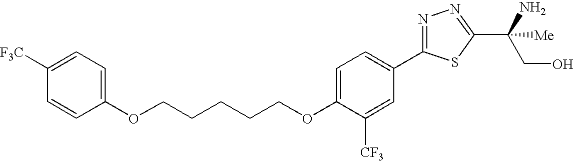 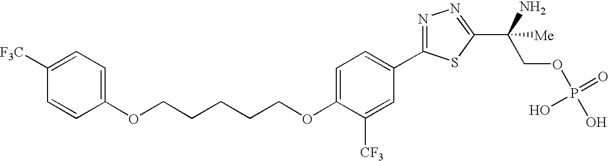 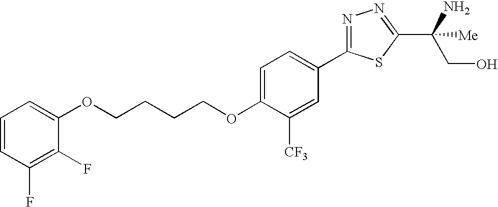 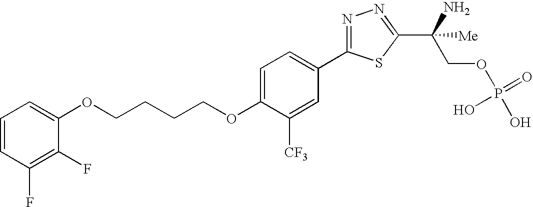 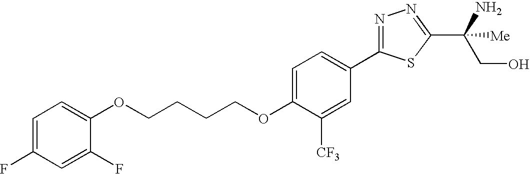 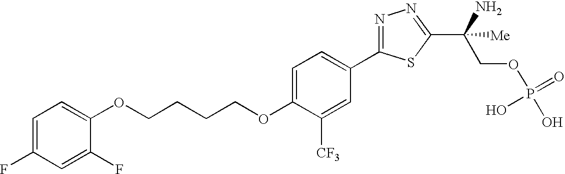 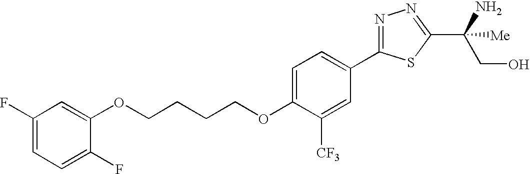 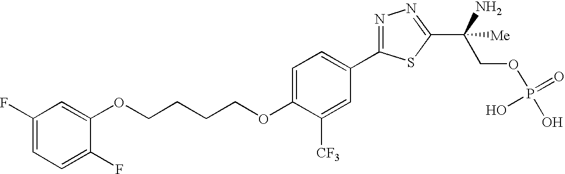 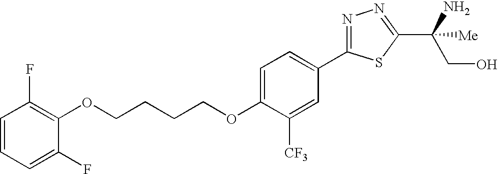 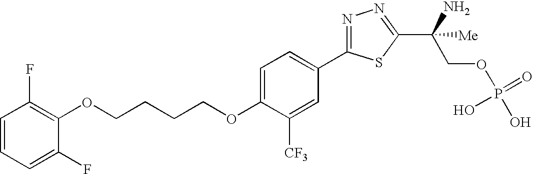 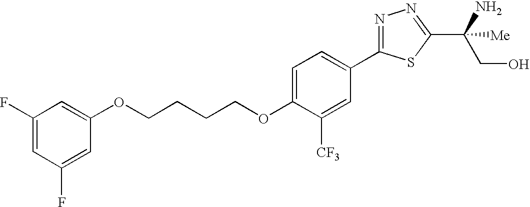 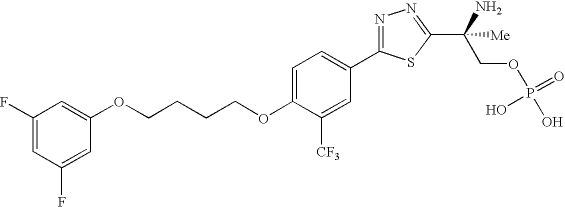 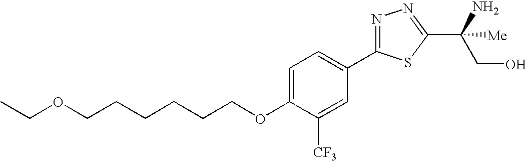 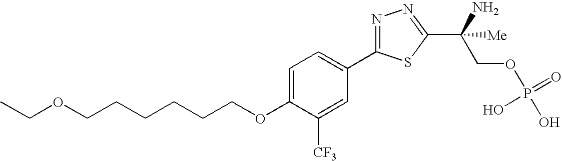 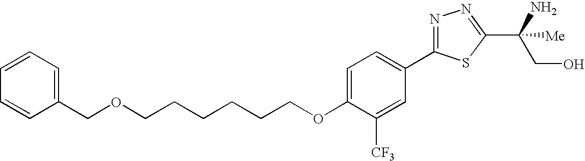 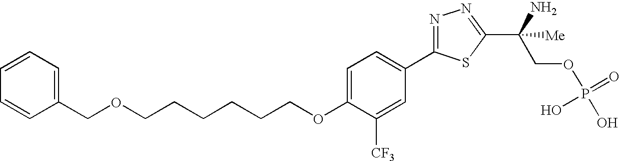 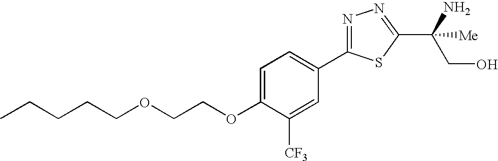 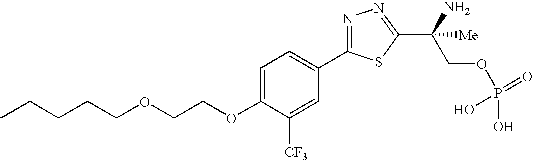 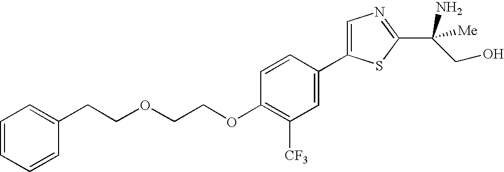 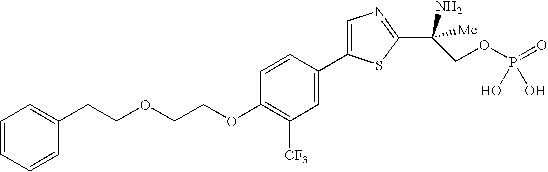 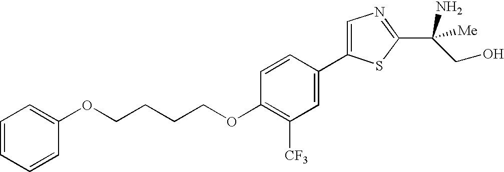 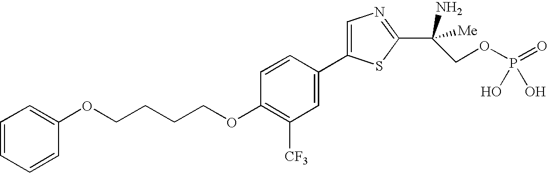 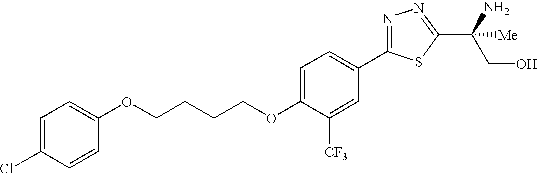 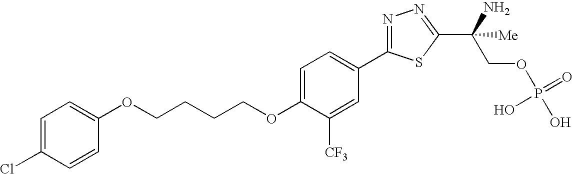 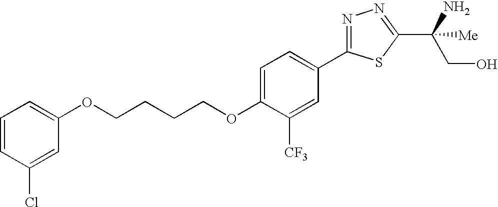 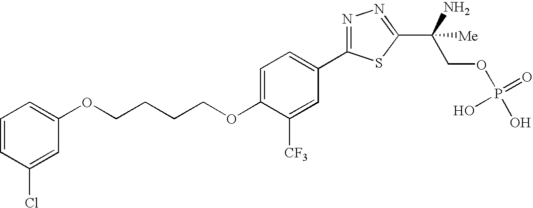 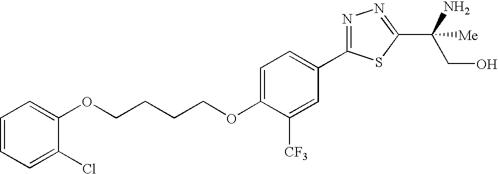 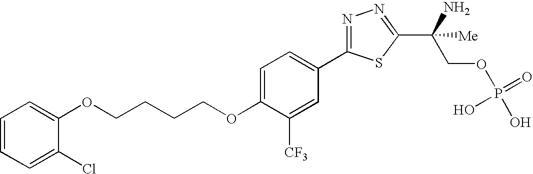 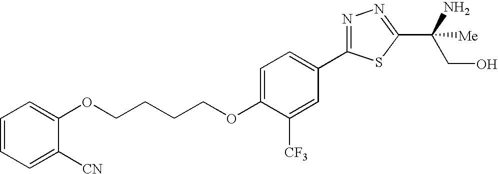 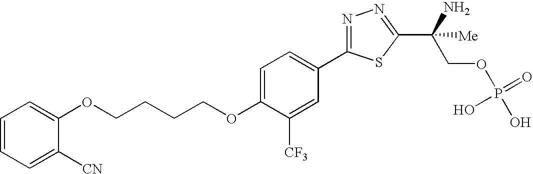 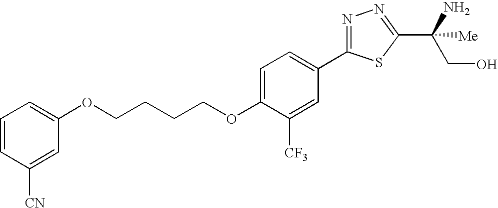 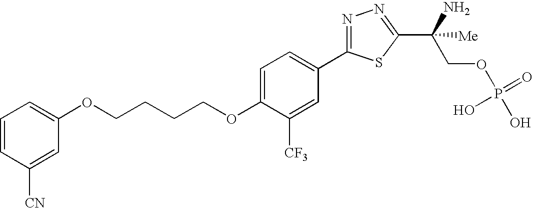 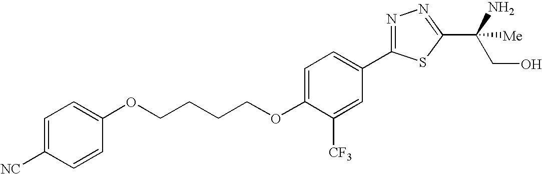 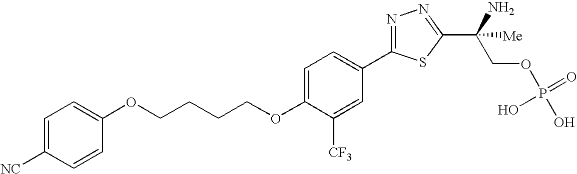 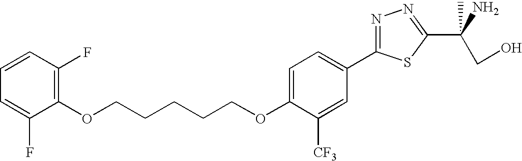 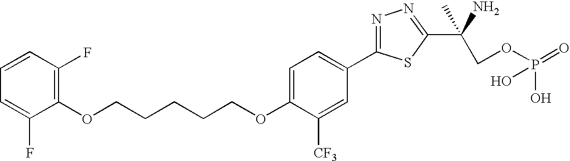 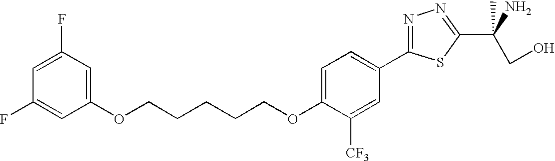 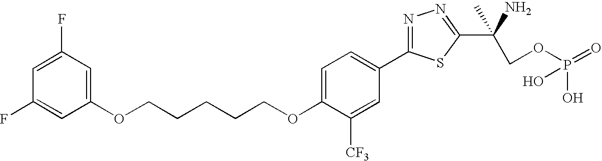 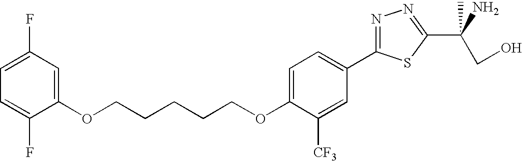 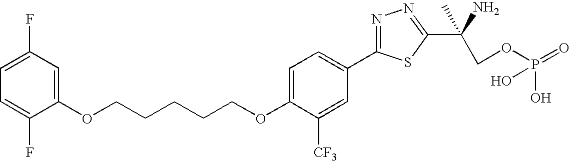 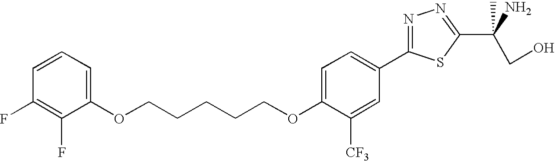 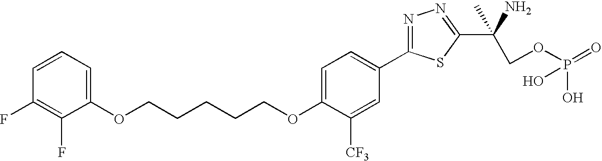 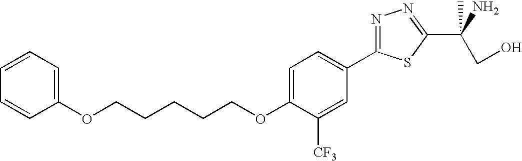 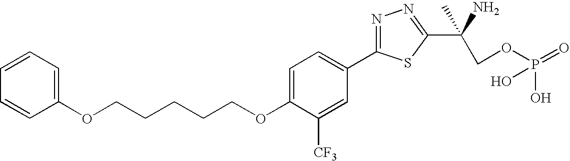 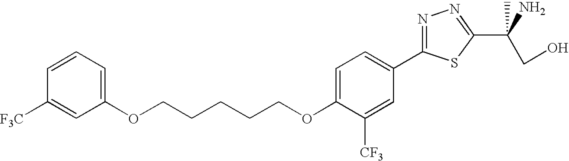 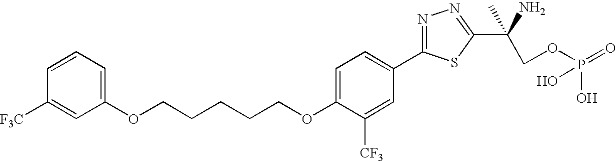 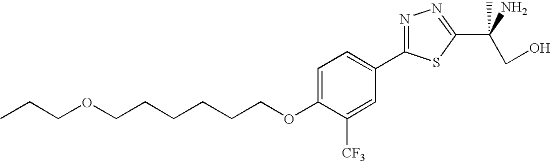 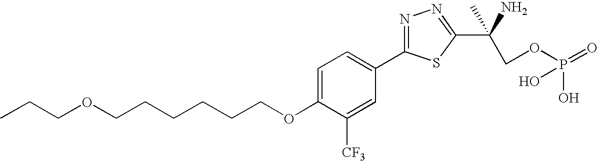 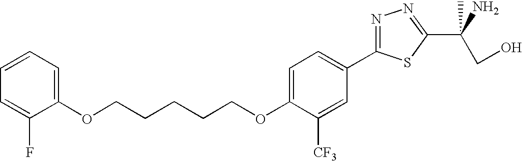 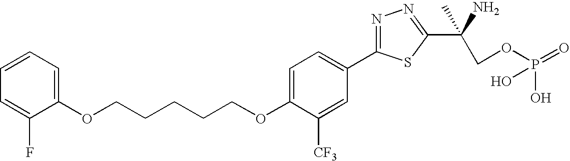 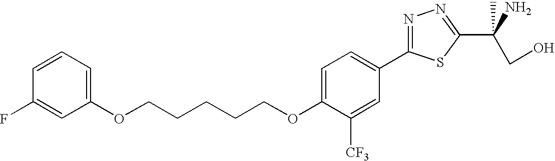 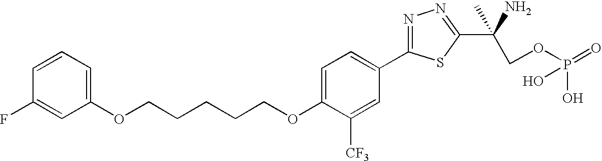 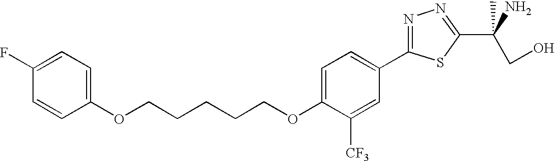 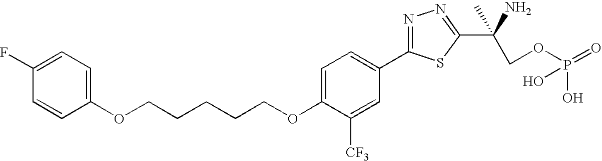 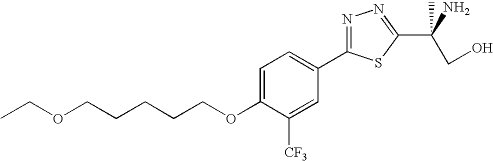 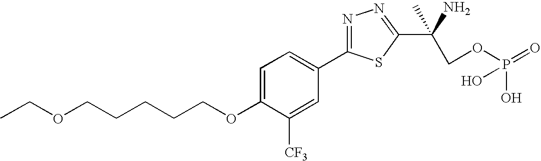 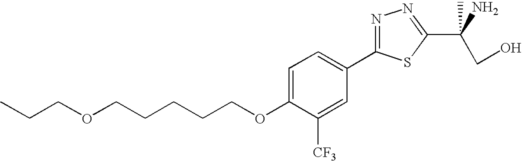 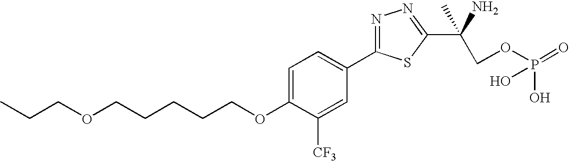 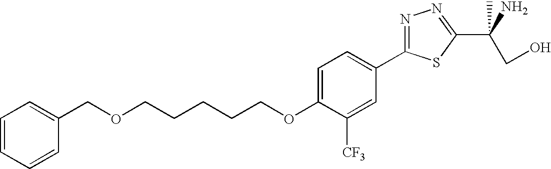 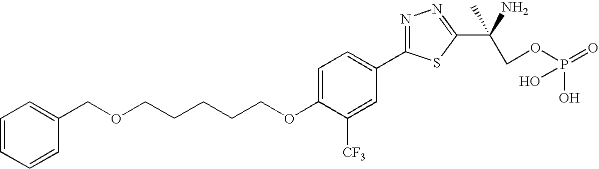 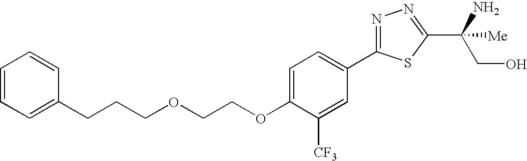 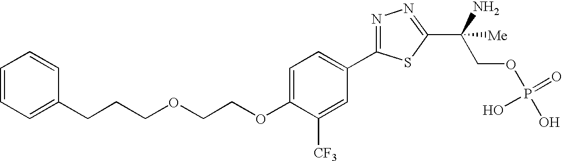 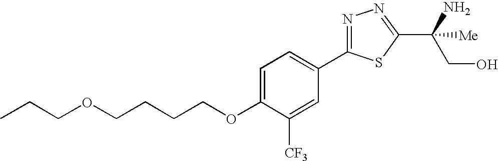 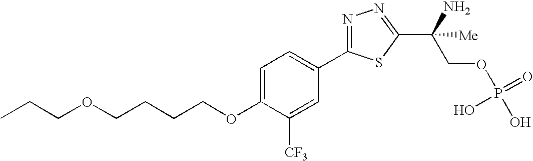 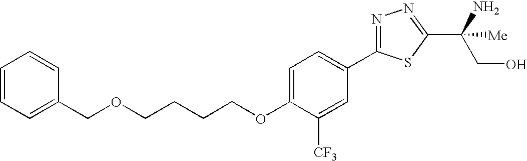 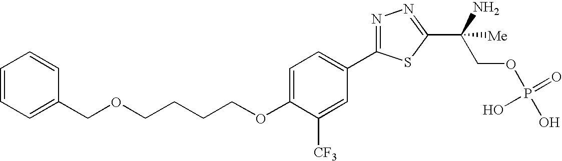 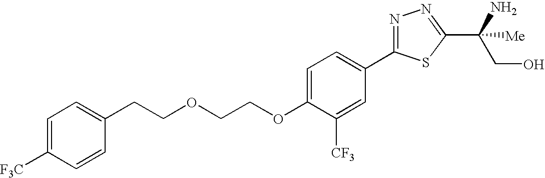 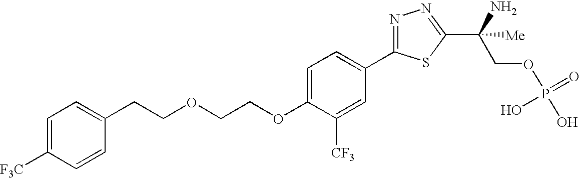 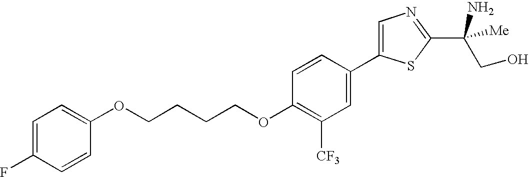 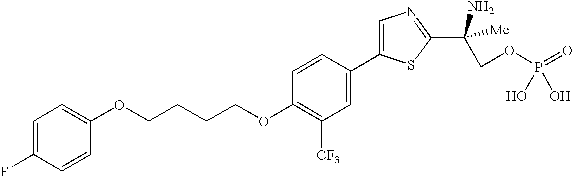 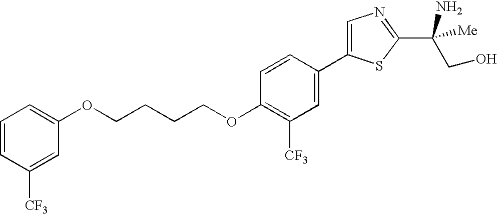 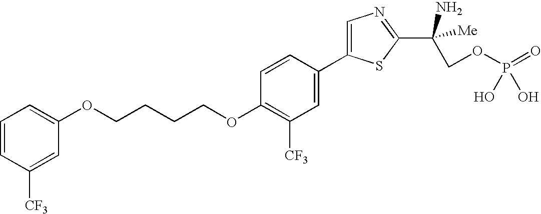
or pharmaceutically acceptable salts, phosphate derivatives, phosphate mimics, or phosphate precursor analogs thereof.

4. A compound of formula III 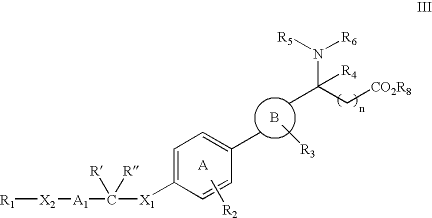
or a pharmaceutically acceptable salt thereof, wherein:
R₁ is alkyl, aryl, heteroaryl, cycloalkyl, heterocycloalkyl, aralkyl, heteroalkyl, or alkyl, any of which may be optionally substituted on carbon with 1, 2, or 3 groups selected from halo, alkyl, haloalkyl, —CF₃, —CN, —OH, or —O-alkyl;
A₁ is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
A₂ is absent or is (C₁-C₁₀)alkylene, (C₂-C₁₀)alkenylene, or (C₂-C₁₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
X₁ is a bond or is CH₂, O, CH₂O, S, —S(O), —S(O)₂, —C(O)—, —C(O)O—, or NRₓ, wherein Rₓ is H or (C₁-C₆)alkyl;
X₂ is O, CH₂O, S, —S(O), —S(O)₂, —C(O)—, —C(O)O—, or NRₓ, wherein Rₓ is H or (C₁-C₆)alkyl;
R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from 0 NH, N-alkyl, SO, or SO₂, any of which may be optionally substituted on carbon with alkyl or halogen
R₂ is cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is absent, hydrogen, halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl; 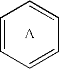
is phenyl or pyridyl; 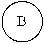
is aryl, heteroaryl, heterocyclo, or cycloalkyl, any of which may be optionally substituted on carbon with 1, 2, or 3 groups selected form halogen, alkyl, O-alkyl, CO₂H, CO₂alkyl, halogen, amino, alkylamino, dialkylamino, —O-alkyl, alkylene-O-alkyl, alkylene-OH, or alkylene-CO₂H;
R₄ is hydrogen, cyano, alkyl, aryl, heteroaryl, alkylene-O-alkyl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅ and R₆, together with the nitrogen to which they are attached, may form a 3, 4, 5, or 6-membered saturated or unsaturated ring, optionally containing 1 or 2 additional heteroatoms selected from O, S, NE, or N-alkyl, and optionally substituted on carbon with halogen, alkyl, hydroxyl, or alkoxy;
n is 0, 1, or 2;
R₈ is hydrogen, alkyl, or aryl.

5. A compound of any of claims 1, 2 or 4, wherein R₂ is alkyl substituted with 1, 2 or 3 halo groups.

6. A compound of any of claims 1, 2 or 4, wherein R₂ is trifluoromethyl.

7. A compound which is (S)-2-Amino-2-(5-(4-(2-(pentyloxy)ethoxy)-3-(trifluoromethyl)phenyl)-1,3,4-thiadiazol-2-yl)propan-1-ol or a pharmaceutically acceptable salt, phosphate derivative, phosphate mimic, or a phosphate precursor analog thereof.

8. A compound which is (S)-2-Amino-2-(5-(4-(2-(pentyloxy)ethoxy)-3-(trifluoromethyl)phenyl)-1,3,4-thiadiazol-2-yl)propyl dihydrogen phosphate or a pharmaceutically acceptable salt thereof.

9. A method for treating a sphingosine associated disorder in a subject comprising administering to the subject a pharmaceutically acceptable amount of a compound as defined in any one of claims 1 to 8 or a pharmaceutically acceptable salt, phosphate derivative, phosphate mimic, or phosphate precursor analog thereof, such that the sphingosine associated disorder is treated.

10. A method for treating multiple sclerosis in a subject comprising administering to the subject a pharmaceutically acceptable amount of a compound as defined in any one of claims 1 to 8 or a pharmaceutically acceptable salt, phosphate derivative, phosphate mimic, or phosphate precursor analog thereof, such that the multiple sclerosis is treated.

11. A pharmaceutical composition comprising a compound as defined in any one of claims 1 to 8 or a pharmaceutically acceptable salt, phosphate derivative, phosphate mimic, or a phosphate precursor analog thereof.

12. A process for the preparation of a pharmaceutical composition according to claim 11.

13. A process for the preparation of a compound as defined in any one of claims 1 to 8 or a pharmaceutically acceptable salt, phosphate derivative, phosphate mimic, or phosphate precursor analog thereof.
